(19) United States
(12) Patent Application Publication
(10) Pub. No.: US 2021/0268072 A1
(43) Pub. Date: Sep. 2, 2021

(54) TATk-CDKL5 Fusion Proteins, Compositions, Formulations, and Use Thereof

(71) Applicants: Alma Mater Studiorum - Universitá di Bologna, Bologna (IT); Amicus Therapeutics, Inc., Cranbury, NJ (US)

(72) Inventors: Elisabetta Ciani, Bologna (IT); Franco Laccone, Stockerau (AT); Sean Clark, Montgomery Township, NJ (US)

(21) Appl. No.: 16/314,145

(22) PCT Filed: Jun. 28, 2017
(86) PCT No.: PCT/US17/39692

§ 371 Date: Dec. 28, 2018

Related U.S. Application Data
(60) Provisional application No. 62/355,579, filed on Jun. 28, 2016.
(60) Provisional application No. 62/381,886, filed on Aug. 31, 2016.

(51) Int. Cl. A61K 38/45; C12N 9/12; A61K 9/00; A61P 25/00

(52) U.S. Cl. CPC A61K 38/45; C12N 9/12; C07K 2319/10; A61K 9/0019; A61P 25/00; C12Y 207/11022

(57) ABSTRACT

Disclosed herein are compositions and formulations containing a TATκ-CDKL5 fusion protein. Also disclosed are methods of producing a TATκ-CDKL5 fusion protein from vectors containing a TATκ-CDKL5 cDNA and methods of transducing cells with the vectors containing a TATκ-CDKL5 cDNA and the TATκ-CDKL5 fusion protein. Also disclosed are uses of TATκ-CDKL5 fusion proteins for treating CDKL5 deficiencies by systemic or intravenous administration of the fusion proteins.

### BACKGROUND

Cyclin-dependent kinase-like 5 (CDKL5) mutation/deficiency, also known as atypical Rett syndrome, is a debilitating postnatal neurological disorder that occurs worldwide in 1 of every 17,000 to 38,000 female births. Males are also affected at a lower incidence. This disorder is not limited to ethnic or racial origin. Symptoms of CDKL5 mutation/deficiency range from mild to severe and present as early onset seizure, cognitive disability, hypotonia as well as autonomic, sleep and gastrointestinal disturbances. Symptoms of disease result from the deficiency of a functional CDKL5 protein.

Mutations in the X-linked CDKL5 gene or deficiencies in the CDKL5 protein in individuals are implicated in the development of atypical or congenital Rett syndrome. See Bertani et al., J. biol. Chem. 2006, 281:32048-320 56, Scala et al., J. Med. Gen., 2005. 42:103-107, and Kalscheuer et al., Am. J. Hum. Genet. 2003. 72:1401-1411. The CDKL5 gene is located on the X-chromosome and encodes a protein that is essential for normal brain development and function. CDKL5 protein is a multifunctional protein that has multiple effects in a neuronal cell. For example, CDKL5 can act as a kinase and phosphorylate MeCP2. MeCP2 is the target of non-atypical Rett syndrome. Girls affected by the CDKL5 mutations or deficiencies typically have a normal prenatal history; irritability and drowsiness in the perinatal period; early-onset epilepsy with onset before 5 months of age, Rett-like features, including deceleration of head growth, stereotypies, poor to absent voluntary hand use, and sleep disturbances, and severe mental retardation with poor eye contact and virtually no language. See Bahi-Buisson and Bienvenu. 2012. Mol. Syndromol. 2:137-152.

Current treatments for CDKL5 mutations/deficiencies are primarily focused on managing symptoms. However, there are currently no treatments that improve the neurological outcome of subjects with CDKL5 mutations or deficiencies. As such, there exists a need for development of therapies for treating the CDKL5 mutations and deficiencies.

### SUMMARY

Described herein are fusion proteins having a CDKL5 polypeptide sequence, wherein the CDKL5 polypeptide sequence has about 50% to 100% sequence identity to SEQ ID NO: 2 or SEQ ID NO: 16, and a TATκ polypeptide sequence, wherein the TATκ polypeptide sequence has about 90% to about 100% sequence identity to SEQ ID NO: 4, wherein the TATκ polypeptide is operatively coupled to the CDKL5 polypeptide. In some aspects, the CDKL5 polypeptide sequence has at least 98%, at least 99% or at least 99.5% sequence identity to SEQ ID NO: 2 or SEQ ID NO: 16. In some aspects, the fusion protein can contain an Igk-chain leader sequence polypeptide, wherein the Igk-chain leader sequence is operatively coupled to the CDKL5 polypeptide. In further aspects, the fusion protein can contain a reporter protein polypeptide, wherein the reporter protein polypeptide is operatively coupled to the CDKL5 polypeptide. In other aspects, the fusion protein can contain a protein tag polypeptide, wherein the protein tag polypeptide is operatively coupled to the CDKL5 polypeptide. In some aspects, the fusion proteins can increase neurite growth, elongation, dendritic spine number, branch number, or branch density in a brain of a subject as compared to a control. In other aspects, the fusion proteins can reduce neuronal apoptosis in the brain of a subject as compared to a control. In some aspects the fusion protein can have a polypeptide sequence according to SEQ ID NO: 8, SEQ ID NO: 10, SEQ ID NO: 12, or SEQ ID NO: 14.

Also provided herein are pharmaceutical formulations containing a therapeutically effective amount of a fusion protein having a CDKL5 polypeptide sequence, wherein the CDKL5 polypeptide sequence has about 50% to 100% sequence identity to SEQ ID NO:2 or SEQ ID NO: 16, and a TATκ polypeptide sequence, wherein the TATκ polypeptide sequence has about 90% to about 100% sequence identity to SEQ ID NO: 4, wherein the TATκ polypeptide is operatively coupled to the CDKL5 polypeptide and a pharmaceutically acceptable carrier. In some aspects, the CDKL5 polypeptide sequence has at least 98%, at least 99% or at least 99.5% sequence identity to SEQ ID NO: 2 or SEQ ID NO: 16. In some aspects the fusion protein contained in the pharmaceutical formulations can contain an Igk-chain leader sequence polypeptide, wherein the Igκ-chain leader sequence is operatively coupled to the CDKL5 polypeptide. In some aspects, the fusion protein contained in the pharmaceutical formulations can contain a reporter protein polypeptide, wherein the reporter protein polypeptide is operatively coupled to the CDKL5 polypeptide. In further aspects, the fusion protein contained in the pharmaceutical formulations can have a polypeptide sequence according to SEQ ID NO: 8, SEQ ID NO: 10, SEQ ID NO: 12, or SEQ ID NO: 14. In further aspects, the therapeutically effective amount of the fusion protein can treat one or more symptoms of a CDKL5 deficiency, Rett syndrome, or Rett syndrome variant in a subject as compared to a control. In additional aspects, the therapeutically effective amount of the fusion protein can increase neurite growth, elongation, dendritic spine number, branch number, or branch density in a brain of a subject as compared to a control. In other aspects, the therapeutically effective amount of the fusion protein can reduce neuronal apoptosis in the brain of a subject as compared to a control. In additional aspects, the therapeutically effective amount of the fusion protein can improve motor function in a subject as compared to a control. In some aspects, the therapeutically effective amount of the fusion protein can improve cognitive function in a subject as compared to a control. In additional aspects, the therapeutically effective amount of the fusion protein can increase neural activity in the visual cortex of a subject as compared to a control.

Provided herein are methods of administering to a subject in need thereof a therapeutically effective amount of a pharmaceutical formulation containing an amount of a fusion protein, where the fusion protein contains a CDKL5 polypeptide sequence, wherein the CDKL5 polypeptide sequence has about 50% to 100% sequence identity to SEQ ID NO:2 or SEQ ID NO: 16 and a TATκ polypeptide sequence, wherein the TATκ polypeptide sequence has about 90% to about 100% sequence identity to SEQ ID NO: 4, wherein the TATκ polypeptide is operatively coupled to the CDKL5 polypeptide, and a pharmaceutically acceptable carrier. In some aspects, the subject in need thereof has or is suspected of having a CDKL5 deficiency, Rett syndrome, or a Rett syndrome variant. In some aspects, the CDKL5 polypeptide sequence has at least 98%, at least 99% or at least 99.5% sequence identity to SEQ ID NO: 2 or SEQ ID NO: 16. In other aspects of the method of administering a therapeutically effective amount of the pharmaceutical formulation, the therapeutically effective amount of the fusion protein can treat one or more symptoms of a CDKL5 deficiency, Rett syndrome, or Rett syndrome variant in a subject as compared to a control.

Also provided herein are uses of fusion proteins for the treatment of a CDKL5 deficiency, Rett syndrome, or Rett syndrome variant, by systemically administering the fusion protein or a pharmaceutical formulation comprising the fusion protein. In some aspects, the fusion protein or pharmaceutical formulation comprising the fusion protein is administered intravenously.

Also provided herein are uses of fusion proteins for increasing neural activity in the visual cortex of a patient having a CDKL5 deficiency, Rett syndrome, or Rett syndrome variant, by systemically administering the fusion protein or a pharmaceutical formulation comprising the fusion protein. In some aspects, the fusion protein or pharmaceutical formulation comprising the fusion protein is administered intravenously.

Also provided herein are uses of fusion proteins for increasing neurite growth, elongation, dendritic spine number, branch number, or branch density in a brain of a patient having a CDKL5 deficiency, Rett syndrome, or Rett syndrome variant, by systemically administering the fusion protein or a pharmaceutical formulation comprising the fusion protein. In some aspects, the fusion protein or pharmaceutical formulation comprising the fusion protein is administered intravenously.

Also provided herein are uses of fusion proteins for reducing neuronal apoptosis in a brain of a patient having a CDKL5 deficiency, Rett syndrome, or Rett syndrome variant, by systemically administering the fusion protein or a pharmaceutical formulation comprising the fusion protein. In some aspects, the fusion protein or pharmaceutical formulation comprising the fusion protein is administered intravenously.

Also provided herein are uses of fusion proteins for improving motor function of a patient having a CDKL5 deficiency, Rett syndrome, or Rett syndrome variant, by systemically administering the fusion protein or a pharmaceutical formulation comprising the fusion protein. In some aspects, the fusion protein or pharmaceutical formulation comprising the fusion protein is administered intravenously.

### BRIEF DESCRIPTION OF THE DRAWINGS

FIG. 1 shows one embodiment of a method to produce a CDKL5 fusion protein, wherein the CDKL5 fusion protein is produced by the cultured cell and secreted into the surrounding culture media.

FIG. 2 shows one embodiment of a method of producing a CDKL5 fusion protein wherein the CDKL5 fusion protein is not secreted into the surrounding cell culture media.

FIG. 3 shows one embodiment of method of delivering a CDKL5 fusion protein to a subject via a transduced or transfected (not specifically shown) autologous cell.

FIGS. 4A and 4B demonstrate western blot analysis results from TATκ-CDKL5₁₁₅ protein expression in transfected HEK 293T cells. TATκ-CDKL5₁₁₅ fusion protein was tagged with an eGFP protein to allow for western blot analysis using an anti-GFP antibody. FIG. 4A demonstrates TATκ-eGFP-CDKL5₁₁₅ fusion protein expression in cell extract from transfected HEK 293T cells. FIG. 4B demonstrates TATκ-eGFP-CDKL5₁₁₅ fusion protein purification from 20× concentrated cell culture medium from TATκ-eGFP-CDKL5₁₁₅-transfected HEK 293T cells.

FIGS. 5A and 5B demonstrate results from a kinase activity assay (FIG. 5A) demonstrating that TAT-eGFP-CDKL5₁₁₅ fusion protein retains CDKL5 autophosphorylation activity. TATκ-eGFP-CDKL5₁₁₅ fusion protein was purified from culture medium on a Ni-NTA resin.

FIG. 6 shows the effect of incubation time on transduction efficiency of one embodiment of a TATκ-eGFP-CDKL5₁₁₅ fusion protein in HEK 293T cells.

FIGS. 7A and 7B shows localization of CDKL5 in TATκ-eGFP-CDKL5₁₁₅ treated HEK 293T cells (FIG. 7B). FIGS. 7A and 7B demonstrate the efficiency of transduction of HEK 293T cells with a TATκ-eGFP-CDKL5₁₁₅ fusion protein as compared to the control (FIG. 7A) (panel on the left) Immunodetection was conducted using an anti-GFP antibody and cells were counterstained with DAPI. The white arrows indicate transduced HEK 293T cells.

FIG. 8 is an image demonstrating a serial of 12 images (1-12) from confocal microscopy demonstrating TATκ-eGFP-CDKL5₁₁₅ transduction into SH-SY5Y cells treated with purified TATκ-eGFP-CDKL5₁₁₅ protein for 30 minutes. Z stack size was 0.4 μm. FIG. 8 demonstrates the efficiency of transduction of SH-SY5Y cells with a TATκ-eGFP-CDKL5₁₁₅ fusion protein.

FIGS. 9A and 9B demonstrate the effect of transduced CDKL5 in neuroblastoma cells (SH-SY5Y) on cell proliferation. TATκ-eGFP-CDKL5₁₁₅ treated cells (FIG. 9B) were observed to have decreased proliferation as compared to TATκ-eGFP (control) treated cells (FIG. 9A). The white arrows indicate mitotic nuclei.

FIG. 10 shows a graph demonstrating the mitotic index of SH-SY5Y cells treated with TATκ-eGFP or TATκ-eGFP-CDKL5₁₁₅ fusion proteins. The y-axis show mitotic cells/total cells as expressed as % TATκ-eGFP. Data are shown as mean±S.E. *** P<0.001 (t-test).

FIG. 11A-11B are images demonstrating a representative phase contrast image of TATκ-eGFP treated (control) SH-SY5Y cells (FIG. 11A), and TATκ-eGFP-CDKL5₁₁₅ treated SH-SY5Y cells (FIG. 11B). Neurite growth was observed to be greater in TATκ-eGFP-CDKL5₁₁₅ treated SH-SY5Y cells as compared to control cells.

FIG. 12 shows a graph demonstrating the quantification of neurite outgrowth of SH-SY5Y cells treated with, TATκ-eGFP fusion protein (control), or TATκ-eGFP-CDKL5₁₁₅. Data is shown as mean±S.E. * P<0.05 (t-test). The y-axis shows neuritic length/cell in microns.

FIGS. 13A-13B show images demonstrating the dendritic morphology and the number of newborn hippocampal granule cells as shown by immunohistochemistry for doublecortin (DCX) in 45-day-old male CDKL5 wild-type (+/Y) (FIG. 13A) and CDKL5 knockout (KO) male mice (−/Y), which are hemizygous (FIG. 13B). Scale bar=50 μm. Abbreviations: GR, granular layer; H, Hilus.

FIGS. 14A-14B show double-fluorescence images of differentiated neuronal precursor cells (NPCs) demonstrating a reduction in the generation and maturation of new neurons (red cells) in neuronal cultures derived from 2-day-old homozygous female CDKL5 knockout mice (−/−) (FIG. 14B) as compared to female wild-type (+/+) (FIG. 14A) neuronal cultures. Cells with a neuronal phenotype are immunopositive for β-tubulin III (red), and cells with an astrocytic phenotype are immunopositive for GFAP (green). Cell nuclei were stained using Hoechst dye (blue). Scale bar=25 μm.

FIG. 15A-15C shows representative images of neuronal precursor cultures generated from cells derived from 2-day-old homozygous CDKL5 knockout mice (−/−) (FIGS. 15B and 15C), transduced with TATκ-eGFP (FIG. 15B) or TATκ-eGFP-CDKL5₁₁₅ (FIG. 15C), as well as neuronal precursor cultures from wild-type mice (+/+) (FIG. 15A). Scale bar=20 μm.

FIG. 16 shows a graph demonstrating quantification of neural maturation as measured by the total neuritic length of differentiated neurons (neurons positive for beta-tubulin III) in neuron precursor cultures derived from newborn (2-day-old) wild-type female (+/+) and homozygous CDKL5 KO (−/−) mice. Cultured and differentiated cells were treated with either TATκ-eGFP or TATκ-eGFP-CDKL5₁₁₅. Values represent mean±SE**p<0.01 as compared to wild-type condition; #p<0.01 as compared to untreated KO samples (Bonferroni test after ANOVA).

FIGS. 17A-17F show images demonstrating immunodetection of CDKL5 in the brains of mice (postnatal day 7) systemically treated (one single sub-cutaneous injection) with the concentrated culture medium (vehicle) (FIGS. 17A and 17D), TATκ-eGFP (FIGS. 17B and 17E), and TATκ-eGFP-CDKL5₁₁₅ (FIGS. 17C and 17F). Samples were collected 4 hours post injection. FIGS. 17D-17F illustrate magnifications of the dotted boxes in FIG. 17A-17C, respectively. Localization of TATκ-eGFP-CDKL5₁₁₅ and TATκ-eGFP in the brain was evaluated by immunohistochemistry using an anti-GFP antibody (red). Images were taken at the level of the sensory-motor cortex. Scale bar=60 μm (lower magnification) and 20 μm (higher magnification).

FIGS. 18A-18D show images of cerebellar sections demonstrating immunodetection of TATκ-eGFP-CDKL5₁₁₅ in the brains of mice (postnatal day 7) systemically treated as in FIGS. 17A-17F with the culture medium (vehicle) (FIGS. 18A and 18B) and TATκ-eGFP-CDKL5₁₁₅ (FIGS. 18C and 18D). Samples were collected 4 hours post injection. Localization of TATκ-eGFP-CDKL5₁₁₅ in the brain was evaluated by immunohistochemistry using an anti-GFP antibody (FIG. 18A-18B). Slides were mounted with DAPI to stain cell nuclei (FIG. 18B-18C). Abbreviations: EGL, external granular layer; IGL, internal granular layer; ML, molecular layer; PL, Purkinje layer. Scale bar=60 μm.

FIG. 19 demonstrates the placement of the cannula for the intraventricular administration of the TATκ-eGFP-CDKL5₁₁₅ fusion protein to mice.

FIG. 20 shows a cartoon depicting the implant and the fusion protein injection schedule for the study demonstrated in FIGS. 21-34. The mice are 4-6 months old at the time of implantation.

FIGS. 21A-21C show images of hippocampal dentate gyrus sections immunostained for DCX demonstrating reduced neurite length and number of newborn granule cells in CDKL5 knockout male mice (−/Y) as compared to male wild-type mice (+/Y) (FIGS. 21B and 21A, respectively). TATκ-eGFP-CDKL5₁₁₅ fusion protein administered intraventricularly on five consecutive days was observed to increase neurite length and number of newborn granule cells in male CDKL5 knockout mice (FIG. 21C) to levels similar to wild-type (FIG. 21A). Scale bar=70 μm.

FIGS. 22A-22C illustrate magnifications of the images in FIG. 21 at the level of the granule layer of the dentate gyrus. Scale bar=25 μm.

FIGS. 23A-23C show examples of the reconstructed dendritic tree of newborn granule cells of CDKL5 wild-type (WT) male mice (also referred to herein as CDKL5+/Y or +/Y) (FIG. 23A), CDKL5 knockout (KO) male mice (also referred to herein as CDKL5 −/Y or −/Y) (FIG. 23B), and CDKL5 KO male mice treated with a TATκ-eGFP-CDKL5₁₁₅ fusion protein via intraventricular injections given once a day for 5 consecutive days (−/Y+TATκ-eGFP-CDKL5₁₁₅) (FIG. 23C).

FIGS. 24A-24B show graphs demonstrating quantification of the mean total dendritic length (FIG. 24A), and mean number of dendritic segments (FIG. 24B) of newborn granule cells (DCX-positive cells) of the dentate gyrus of CDKL5 WT male mice (+/Y), CDKL5 KO male mice (−/Y), and CDKL5 KO male mice treated with TATκ-eGFP-CDKL5₁₁₅ fusion protein via intraventricular injections given once a day for 5 consecutive days (−/Y+TATκ-eGFP-CDKL5). Values represent mean±SE. ** p<0.01; *** p<0.001 as compared to +/Y; #p<0.05 as compared to the −/Y samples (Bonferroni's test after ANOVA).

FIGS. 25A-25B show graphs demonstrating quantification of the mean length (FIG. 25A) and mean number (FIG. 25B) of branches of the different orders of newborn granule cells of the dentate gyrus of CDKL5 wild-type male mice (+/Y), CDKL5 KO male mice (−/Y), and CDKL5 KO male mice treated with TATκ-eGFP-CDKL5₁₁₅ fusion protein via intraventricular injections given once a day for 5 consecutive days (−/Y+TATκ-eGFP-CDKL5). Values represent mean±SE. * p<0.05; ** p<0.01 as compared to +/Y; #p<0.05 as compared to the −/Y samples (Bonferroni's test after ANOVA).

FIG. 26 shows a graph demonstrating quantification of apoptotic cells (caspase-3 positive cells) in CDKL5 wild-type male mice (+/Y), CDKL5 KO male mice (−/Y), and CDKL5 KO male mice treated with TATκ-eGFP-CDKL5₁₁₅ fusion protein via intraventricular injections given once a day for 5 consecutive days (−/Y+TATκ-eGFP-CDKL5). Values represent mean±SE. * P<0.05 as compared to +/Y; #p<0.05 as compared to the −/Y samples (Bonferroni's test after ANOVA).

FIG. 27 shows a graph demonstrating quantification of the number of DCX positive cells in the DG of CDKL5 wild-type male mice (+/Y), CDKL5 KO male mice (−/Y), and CDKL5 KO male mice treated with TATκ-eGFP-CDKL5₁₁₅ fusion protein via intraventricular injections given once a day for 5 consecutive days (−/Y+TATκ-eGFP-CDKL5). Data are expressed as number of cells/mm2*p<0.05 as compared to +/Y; #p<0.05 as compared to the −/Y samples (Bonferroni's test after ANOVA).

FIGS. 28A-28C show representative images demonstrating brain sections processed for synaptophysin (SYN) immunofluorescence from the molecular layer of the dentate gryrus (DG) from a CDKL5 wild-type male mouse (+/Y) (FIG. 28A), a CDKL5 KO male mouse (−/Y) (FIG. 28B), and a CDKL5 KO male mouse treated with TATκ-eGFP-CDKL5₁₁₅ fusion protein via intraventricular injections given once a day for 5 consecutive days (−/Y+TATκ-eGFP-CDKL5) (FIG. 28C). Scale bare=80 μm. Abbreviation: GR, granular layer; Mol, molecular layer.

FIGS. 29A-29C show representative images demonstrating brain sections processed for phospho-AKT (P-AKT) immunofluorescence from the molecular layer of the dentate gryrus (DG) from a CDKL5 wild-type male mouse (+/Y) (FIG. 29A), a CDKL5 KO male mouse (−/Y) (FIG. 29B), and a CDKL5 KO male mouse treated with TATκ-eGFP-CDKL5₁₁₅ fusion protein via intraventricular injections given once a day for 5 consecutive days (−/Y+TATκ-eGFP-CDKL5) (FIG. 29C). Scale bare=80 μm. Abbreviation: GR, granular layer; Mol, molecular layer.

FIGS. 30A-30B show graphs demonstrating the quantification of synaptophysin (SYN) optical density in the molecular layer of the hippocampus (FIG. 30A) and layer III of the cortex (FIG. 30B) in CDKL5 wild-type male mice (+/Y), CDKL5 KO male mice (−/Y), and CDKL5 KO male mice treated with TATκ-eGFP-CDKL5₁₁₅ fusion protein via intraventricular injections given once a day for 5 consecutive days (−/Y+TAT-eGFP-CDKL5). Data are given as fold difference vs. the corresponding zone of the molecular layer or cortex of wild-type mice. Values represent mean±SD. **p<0.01; ***p<0.001 as compared to +/Y; #p<0.05 as compared to the −/Y samples (Bonferroni's test after ANOVA).

FIGS. 31A-31B show graphs demonstrating the quantification of the optical density of Ser437 phosphorylated-AKT (PAKT) in the molecular layer of the hippocampus (FIG. 31A) and layer V of the cortex (FIG. 31B) in CDKL5 wild-type male mice (+/Y), CDKL5 KO male mice (−/Y), and CDKL5 KO male mice treated with TATκ-eGFP-CDKL5₁₁₅ fusion protein via intraventricular injections given once a day for 5 consecutive days (−/Y+TATκ-eGFP-CDKL5). Data are given as fold difference vs. the corresponding zone of the molecular layer or cortex of wild-type mice. Values represent mean±SD. **p<0.01 as compared to +/Y; #p<0.01 as compared to the −/Y samples (Bonferroni's test after ANOVA).

FIG. 32 shows a graph demonstrating dendritic mean total dendritic length of Golgi-stained granule cells in CDKL5 wild-type male mice (+/Y) and CDKL5 KO male mice (−/Y) treated with a vehicle or TATκ-eGFP-CDKL5₁₁₅ via intraventricular injections given once a day for 5 consecutive days. On the right scheme of a hippocampal slice showing the different layers and the position of CA1 pyramidal cells and granule cells. The molecular layer (Mol) of the dentate gyrus (DG) contains the granule cell dendrites. Values represent mean±SE. **p<0.01 as compared to +/Y; #p<0.05 as compared to the −/Y samples (Bonferroni's test after ANOVA).

FIG. 33 shows images of Golgi-stained dendritic branches of granule cells of CDKL5 wild-type male mice (+/Y) and CDKL5 KO male mice (−/Y) treated with vehicle or TATκ-eGFP-CDKL5₁₁₅ via intraventricular injections given once a day for 5 consecutive days.

FIG. 34 shows a graph demonstrating the Quantification of number of dendritic spines of granule cells of CDKL5 wild-type male mice (+/Y) and CDKL5 KO male mice (−/Y) treated with vehicle or TATκ-eGFP-CDKL5₁₁₅ via intraventricular injections given once a day for 5 consecutive days. Values represent mean±SE. ** p<0.01 as compared to +/Y; #p<0.05 as compared to the −/Y samples (Bonferroni's test after ANOVA).

FIG. 35 shows a cartoon depicting the implant and the fusion protein injection schedule for the behavioral study demonstrated in FIGS. 36-38. The mice are 4-6 months old at the time of implantation.

FIG. 36 shows a graph demonstrating the quantification of the learning phase as determined via the Morris Water Maze test in CDKL5 wild-type male mice (+/Y; n=8), CDKL5 KO male mice (−/Y; n=8), and CDKL5 KO male mice treated with a TATκ-eGFP-CDKL5₁₁₅ fusion protein (−/Y+TATκ-eGFP-CDKL5; n=6). Values represent mean±SE. * P<0.05, ** P<0.01 as compared to the untreated wild-type condition and #P<0.01 as compared to the untreated CDKL5 knockout condition as tested with Fisher LSD after ANOVA.

FIGS. 37A-37B show graphs demonstrating memory ability as determined by a passive avoidance test in CDKL5 wild-type male mice (+/Y; n=8), CDKL5 KO male mice (−/Y; n=8), and CDKL5 KO male mice treated with a TATκ-eGFP-CDKL5₁₁₅ fusion protein (−/Y+TATκ-eGFP-CDKL5; n=6). Graphs show the latency time for entering the dark compartment on the first day (FIG. 37A) and on the second day (FIG. 37B) of the behavioral procedure. Values represent mean±SE. *** P<0.001 as compared to the untreated wild-type condition and #P<0.01 as compared to the untreated CDKL5 knockout mice as tested with Fisher LSD after ANOVA.

FIGS. 38A-38B show a graph demonstrating quantification of motor ability as determined by a clasping test in which total amount of time spent limb clasping during a 2 minute interval was measured in CDKL5 wild-type male mice (+/Y; n=8), CDKL5 KO male mice (−/Y; n=8), and CDKL5 KO male mice treated with a TATκ-eGFP-CDKL5₁₁₅ fusion protein (−/Y+TATκ-CDKL5; n=8) according to the injection schedule in FIG. 35. Values represent mean±SD. ***p<0.001 as compared to +/Y; #p<0.001 as compared to the −/Y samples (Bonferroni's test after ANOVA).

FIG. 39 demonstrates body weight (in grams) of CDKL5 wild-type male (+/Y) and CDKL5 knockout male (−/Y) mice treated with a TATκ-eGFP-CDKL5₁₁₅ fusion protein according to the treatment schedule of FIG. 20 (+/Y; n=8) or FIG. 35 (−/Y; n=6). Mice were left to recover for 7 days after cannula implantation.

FIGS. 40A-40F demonstrate a comparison of Allograft Inflammatory Factor 1 (AIF-1) staining in untreated animals and treated with TATκ-eGFP-CDKL5₁₁₅ for 5 days or 10 days by intraventricular injection. Mice receiving injections were 4-6 months old.

FIG. 41 shows an image of a western blot demonstrating secretion (lanes 1-4) and expression (lanes 5-8) of a TATκ-eGFP-CDKL5₁₁₅ or TATκ-eGFP-CDKL5₁₀₇ fusion protein in HEK 293T cells cultured in DMEM and a comparison of the expression and secretion pattern of TATκ-eGFP-CDKL5₁₁₅ and TATκ-eGFP-CDKL5₁₀₇. Extracts were produced from HEK 293T cells or medium from cultured HEK 293T cells that were transiently transfected. About 15 μg total protein extracts for HEK 293T cells were loaded on the gel, 20 μL of a 40× concentrated DMEM medium was loaded to show protein secretion. The arrows indicate the respective CDKL5 fusion proteins.

FIGS. 42A-42B show graphs demonstrating CDKL5 fusion protein stability in HEK 293T (FIG. 42A) and neuroblastoma cells (FIG. 42B). HEK 293T or neuroblastoma cells were transfected with a plasmid containing polynucleotides encoding TATκ-eGFP-CDKL5₁₁₅ or TATκ-eGFP-CDKL5₁₀₇. Twenty-four hours later cells were incubated with cycloheximide (Chx; 50 μg/ml) for the indicated times (3, 6 or 8 hours). Ectopically expressed CDKL5 was detected by CDKL5 immunoblotting.

FIG. 43 shows an image of a western blot demonstrating protein stability of TATκ-eGFP-CDKL5₁₁₅ or TATκ-eGFP-CDKL5₁₀₇ in HEK 293T cells. HEK 293T cells were transfected with TATκ-eGFP-CDKL5₁₁₅ or TATκ-eGFP-CDKL5₁₀₇. Twenty-four hours later cells were incubated with cycloheximide (Chx; 50 μg/ml) for the indicated times (3 and 6 hours).

FIG. 44 shows a graph comparing the effect of TATκ-eGFP-CDKL5₁₁₅, TATκ-CDKL5₁₁₅ and TATκ-eGFP-CDKL5₁₀₇ on neuroblastoma cell proliferation. SH-SY5Y cells were treated with TATκ-eGFP, TATκ-eGFP-CDKL5₁₁₅, TATκ-CDKL5₁₁₅ or TATκ-eGFP-CDKL5₁₀₇. After 24 hours from transfection, the mitotic index was evaluated as number of mitotic cells on total cell number as expressed as % TATκ-eGFP. Note that the two CDKL5 isoforms show a similar activity of inhibition of SH-SY5Y mitosis compared to TATκ-eGFP treated control cells. Values represent mean±SEM. *** p<0.001 as compared to TATκ-eGFP treated cells (t-test).

FIGS. 45A-45D show localization of CDKL5 in TATκ-eGFP-CDKL5₁₀₇ treated hippocampal neuronal cultures. FIGS. 45A and 45C demonstrate the efficiency of transduction and the subcellular localization of a TATκ-eGFP control protein. FIGS. 45B and 45D demonstrate the efficiency of transduction and the subcellular localization of a TATκ-eGFP-CDKL5₁₀₇ protein Immunodetection was conducted using an anti-GFP antibody and cells were counterstained with DAPI. Higher magnification shows an enlargement of a dendrite segment.

FIGS. 46A-46C show images by laser confocal microscopy of a dendrite segment of a hippocampal neuron treated with TATκ-eGFP-CDKL5₁₀₇. Images show co-localization of TATκ-eGFP-CDKL5₁₀₇ protein with a postsynaptic protein (PSD-95). eGFP-CDKL5 immunodetection was conducted using an anti-GFP antibody (FIG. 45A).

FIG. 47 shows a graph demonstrating dendritic length of CDKL5 KO (−/Y) hippocampal neurons after treatment with TATκ-eGFP, TATκ-eGFP-CDKL5₁₁₅ or TATκ-eGFP-CDKL5₁₀₇ fusion proteins. Values represent mean±SEM. *** p<0.001 as compared to CDKL5 wild-type (+/Y) neurons; #p<0.05 as compared to the −/Y neurons (Bonferroni's test after ANOVA).

FIG. 48 shows a graph demonstrating number of synaptophysin puncta in hippocampal neurons of CDKL5 KO (−/Y) hippocampal neurons after treatment with TATκ-eGFP, TATκ-eGFP-CDKL5₁₁₅ or TATκ-eGFP-CDKL5₁₀₇ fusion proteins. Values represent mean±SEM. *** p<0.001 as compared to CDKL5 wild-type (+/Y) neurons; #p<0.05 as compared to the −/Y neurons (Bonferroni's test after ANOVA).

FIGS. 49A-49B show cartoons depicting a treatment schedule and route of administration of the CDKL5 fusion protein. Male CDKL5 wild-type (CDKL5+/Y; +/Y) mice received treatment with TATκ-eGFP (n=6) while KO (CDKL5 −/Y; −/Y) mice were treated with TATκ-eGFP (n=6) or TATκ-eGFP-CDKL5₁₀₇ (n=6) as indicated above. Treatment period consisted of a single daily injection (10 μl injection, approximately 50 ng/injection) for 5 consecutive days, followed by a two day rest period and then 5 additional days of a single injection. There was a total of 10 injections which were done in a 12 day period. Mice were 4-6 months old at the time of implantation. The treatment schedule, route of administration, and age of mice used in FIGS. 49A-49B apply to FIGS. 51-59.

FIG. 50 shows a graph demonstrating results from Morris Water Maze testing after receiving the TATκ-eGFP-CDKL5₁₀₇ fusion protein as described in FIGS. 49A-49B. Values represent mean±SE. * p<0.05, ** p<0.01, as compared to the CDKL5 wild-type mice (+/Y) treated with TATκ-eGFP (+/Y+TATκ-eGFP); #p<0.01 as compared to CDKL5 KO mice (−/Y) treated with TATκ-eGFP (−/Y+TATκ-eGFP) (Fisher LSD test after ANOVA).

FIGS. 51A-51C show graphs demonstrating spatial memory from measuring (FIG. 51A) latency to enter the former platform quadrant, FIG. 51B) frequency of entrances into the former platform quadrant, (FIG. 51C) percentage of time spent in the former platform quadrant. Performance in all parameters was severely impaired in TATκ-eGFP treated CDKL5 KO mice (−/Y+TATκ-eGFP). TATκ-eGFP-CDKL5₁₀₇ treated CDKL5 KO mice (−/Y+TATκ-eGFP-CDKL5₁₀₇) showed statistically significant improvement in all parameters, FIGS. 51A, 51B, and 51C. Values represent mean±SE. * p<0.05, ** p<0.01, *** p<0.001 as compared to the CDKL5 wild-type mice; #p<0.01 as compared to the TATκ-eGFP treated CDKL5 KO mice (Fisher LSD test after ANOVA).

FIGS. 52A-52B show graphs demonstrating the effect of treatment on learning and memory using a passive avoidance (PA) test. After treatment period and a two day rest period, mice received passive avoidance (PA) testing. The experiment utilized a test cage with two chambers (light and dark). On the first day, animals were placed in the light chamber and instinctively move into the dark chamber where they are conditioned with a single adverse event (foot-shock). FIG. 52A indicates that the latency time to enter the dark chamber was similar for all groups. On the second day (testing period) animals are again placed in the light chamber. Memory of the adverse event was measured by latency time to enter the dark chamber and represented in FIG. 52B. TATκ-eGFP treated CDKL5 KO male mice (−/Y+TATκ-eGFP) were severely impaired in this task, as shown by a reduced latency to enter the dark compartment in comparison with CDKL5 wild-type male mice treated with TATκ-eGFP (+/Y+TATκ-eGFP). TATκ-eGFP-CDKL5₁₀₇ treated CDKL5 KO male mice (−/Y+TATκ-eGFP-CDKL5₁₀₇) showed similar latency time as compared to wild-type male mice (FIG. 52B). These differences were statistically significant in comparison to TATκ-eGFP treated CDKL5 KO male mice (−/Y+TATκ-eGFP). ** p<0.01 as compared to the CDKL5 wild-type male condition; #p<0.01 as compared to the TATκ-eGFP treated CDKL5 KO male condition (Fisher LSD test after ANOVA).

FIGS. 53A-53B show (FIG. 53A) a cartoon of a Y-maze used to evaluate the effect of treatment on learning and memory and (FIG. 53B) a graph demonstrating the results from the Y maze test. CDKL5 KO mice treated with TATκ-eGFP-CDKL5₁₀₇ (−/Y+TATκ-eGFP-CDKL5₁₀₇) showed performance similar to CDKL5 wild-type mice treated with TATκ-eGFP (+/Y+TATκ-eGFP). * p<0.05, ** p<0.01 as compared to the wild-type condition; #p<0.05 as compared to the TATκ-eGFP treated CDKL5 KO condition (−/Y+TATκ-eGFP) (Fisher LSD test after ANOVA).

FIGS. 54A-54B show (FIG. 54A) a graph and (FIG. 54B) an image demonstrating clasping (right mouse) vs. unclasping (left mouse) in a hind limb clasping test used to evaluate the effect of treatment on motor function. Treatment of CDKL5 KO mice with TATκ-eGFP-CDKL5₁₀₇ (−/Y+TATκ-eGFP-CDKL5₁₀₇) led to a statistically significant reduction in clasping time as compared to TATκ-eGFP treated CDKL5 KO (−/Y+TATκ-eGFP) Values represent mean±SE. *** p<0.001 as compared to the wild-type condition (+/Y+TATκ-eGFP); ##p<0.001 as compared to the TATκ-eGFP treated CDKL5 KO (Fisher LSD test after ANOVA).

FIGS. 55A and 55B show graphs demonstrating breathing disturbances in treated TATκ-eGFP-CDKL5₁₀₇) and untreated (+TATκ-eGFP) CDKL5 wild-type (+/Y) or CDKL5 KO (−/Y) mice as measured by the number of apneas during non-rapid eye movement (NREM) (FIG. 55A) and rapid eye movement (REM) (FIG. 55B) sleep. Apneas were measured using whole body plethysmography. * p<0.01; ** p<0.01 as compared to the wild-type condition; #p<0.01 as compared to the CDKL5 KO −/Y condition (t-test).

FIGS. 56A-56D show a graph (FIG. 56A) and (FIGS. 56B-56D) reconstructed dendritic trees of newborn granule cells demonstrating the effect of treatment with TATκ-eGFP-CDKL5₁₀₇ fusion protein (+TATκ-eGFP-CDKL5₁₀₇) or TATκ-eGFP (+TATκ-eGFP) on CDKL5 wild-type (+/Y) or CDKL5 KO (−/Y) mice. Values represent mean±SE. ** p<0.01 as compared to the wild-type condition; #p<0.01 as compared to the TATκ-eGFP treated CDKL5 KO condition (Bonferroni test after ANOVA).

FIG. 57 demonstrates quantification of the number of DCX positive cells in the hippocampus (dentate gyrus) of CDKL5 wild-type male mice (+/Y) treated with TATκ-eGFP (+/Y+TATκ-eGFP), CDKL5 KO male mice (−/Y) (−/Y+TATκ-eGFP), and CDKL5 KO male mice treated with TATκ-eGFP-CDKL5₁₀₇ (−/Y+TATκ-eGFP-CDKL5₁₀₇). Treatment period consisted of once daily intraventricular injection for 5 days followed by a two day rest period then an additional 5 injections. Animals were sacrificed 10 days after the last injection. Data are expressed as number of cells/mm ** p<0.01 as compared to +/Y; ##p<0.001 as compared to the CDKL5 KO samples (Bonferroni's test after ANOVA). Data suggest that the positive impact of treatment with TATκ-eGFP-CDKL5 on the number of DCX-positive cells is retained 10 days after treatment completion.

FIG. 58 demonstrates quantification of the total number of cleaved Caspase 3 positive cells in the hippocampus (dentate gyrus) of CDKL5 wild-type male mice (+/Y) treated with TATκ-eGFP (+/Y+TATκ-eGFP), CDKL5 KO male mice (−/Y) (−/Y+TATκ-eGFP), and CDKL5 KO male mice treated with TATκ-eGFP-CDKL5₁₀₇ (−/Y+TATκ-eGFP-CDKL5₁₀₇). The treatment protocol was a once daily intraventricular injection given for 5 days followed by a two day rest period then an additional 5 injections. Animals were sacrificed 10 days after the last injection.

FIG. 59 shows a graph demonstrating body weight of CDKL5 KO (−/Y) and CDKL5 wild-type (+/Y) mice treated with TATκ-eGFP (+TATκ-eGFP) or CDKL5 KO (−/Y) mice treated with or TATκ-eGFP-CDKL5₁₀₇ (−/Y+TATκ-eGFP-CDKL5₁₀₇) via once daily intraventricular injection. Mice were allowed to recover for 7 days after cannula implantation and sacrificed 10 days after the last injection.

FIGS. 60A-60C show images by laser confocal microscopy of a dendrite segment of a hippocampal neuron transduced with TATκ-eGFP-CDKL5₁₀₇. Images show co-localization of TATκ-eGFP-CDKL5₁₀₇ protein with a presynaptic protein (synaptophysin; SYN). eGFP-CDKL5 immunodetection was conducted using an anti-GFP antibody (FIG. 64A). FIGS. 60A-60C were produced using the protocol as described in relation to FIG. 45.

FIG. 61 shows a graph demonstrating dendritic spine number of CDKL5 wild-type (+/Y) or CDKL5 KO (−/Y) hippocampal neurons in Golgi-stained sections after treatment with TATκ-eGFP (+TATκ-eGFP), TATκ-eGFP-CDKL5₁₁₅ (+TATκ-eGFP-CDKL5₁₁₅) or TATκ-eGFP-CDKL5₁₀₇ (+TATκ-eGFP-CDKL5₁₀₇) fusion proteins. Values represent mean±SEM. ** p<0.01 as compared to wild-type (+/Y) neurons; #p<0.05 as compared to the CDKL5 (−/Y) neurons (Bonferroni's test after ANOVA).

FIG. 62 shows a treatment schedule for systemic administration of the CDKL5 fusion proteins. To mimic a daily human dose administration we used an infusion method which is based on a programmable pump implanted under the skin with a refillable reservoir. CDKL5 −/Y mice were treated with TATκ-eGFP or TATκ-eGFP-CDKL5₁₀₇. Protein is injected through a cannula directly into the internal carotid artery. This system allowed us to apply a twice a day infusion protocol (morning and evening; 20 μl injection, approximately 50 ng/injection) for the duration of 10 days. Mice were 4-6 months of age at the time of implantation. The treatment schedule and age of mice described with respect to FIG. 62 apply to FIGS. 63A-72.

FIGS. 63A-63B show images of hippocampal dentate gyrus sections immunostained for DCX. TATκ-eGFP-CDKL5₁₀₇ fusion protein administered systemically on ten consecutive days to mice that were 4-6 months old was observed to increase neurite length and number of newborn granule cells in CDKL5 KO male mice (FIG. 63B).

FIG. 64 shows a graph demonstrating the effect of systemic treatment of 4-6 month old mice with a TATκ-eGFP-CDKL5₁₀₇ fusion protein on breathing disturbances as measured by the number of apneas during NREM sleep.

FIG. 65 is graph showing mean total dendritic length of newborn (doublecortin-positive) granule cells of untreated CDKL5+/Y and CDKL5 −/Y mice, and CDKL5 −/Y mice treated with TATκ-eGFP or TATκ-eGFP-CDKL5₁₀₇. Values represented as means±SE. ** p<0.01; *** p<0.001 as compared to the untreated CDKL5+/Y condition; #p<0.05 as compared to the untreated CDKL5 −/Y samples (Fisher LSD test after ANOVA).

FIG. 66 is a graph showing mean total dendritic length of Golgi-stained granule cells of untreated CDKL5+/Y and CDKL5 −/Y mice, and CDKL5 −/Y mice treated with TATκ-eGFP or TATκ-eGFP-CDKL5₁₀₇. Values represented as means±SE. ** p<0.01; *** p<0.001 as compared to the untreated CDKL5+/Y condition; #p<0.05 as compared to the untreated CDKL5 −/Y samples (Fisher LSD test after ANOVA).

FIG. 67 is a graph showing the number of digging bouts of untreated CDKL5+/and CDKL5 −/Y mice, and CDKL5 −/Y mice treated with TATκ-eGFP or TATκ-eGFP-CDKL5₁₀₇. Values represented as means±SE. ** p<0.01; as compared to the untreated CDKL5+/Y condition; #p<0.05 as compared to the untreated CDKL5 −/Y samples (Fisher LSD test after ANOVA).

FIG. 68 is a graph showing the nest quality of untreated CDKL5+/and CDKL5 −/Y mice, and CDKL5 −/Y mice treated with TATκ-eGFP or TATκ-eGFP-CDKL5₁₀₇. Values represented as means±SE. ** p<0.01; as compared to the untreated CDKL5+/Y condition; #p<0.05 as compared to the untreated CDKL5 −/Y samples (Fisher LSD test after ANOVA).

FIG. 69 are a series of representative images of neural activity in the visual cortex collected at different time points in one CDKL5 −/Y mouse treated with either TATκ-eGFP or TATκ-eGFP-CDKL5₁₀₇.

FIG. 70 is a graph showing the mean amplitude of visually evoked responses measured before and after 6 and 10 days of treatment in CDKL5 −/Y mice treated with either TATκ-eGFP or TATκ-eGFP-CDKL5₁₀₇. The persistence of the effect was evaluated with an additional measurement 6-10 days after treatment cessation (washout). As a reference, the 95% confidence interval of untreated wild-type response amplitude over time is shown in the patterned area. Error bars represent standard error of the mean. Two-way ANOVA (repeated measures for the factor time) revealed a time X treatment interaction p<0.05; post-hoc Holm-Sidak's multiple comparisons test: * p<0.05, **p<0.01.

FIG. 71 is a graph showing the dendritic spine density of the primary visual cortex (V1) pyramidal neurons (layer 2/3) from untreated CDKL5+/Y and CDKL5 −/Y mice, and CDKL5 −/Y mice treated with TATκ-eGFP or TATκ-eGFP-CDKL5₁₀₇ (n=5) and sacrificed at the end of treatment (short term) or with TATκ-eGFP or TATκ-eGFP-CDKL5₁₀₇ and sacrificed 10 days after treatment cessation (long term). Values are represented as means±SE. * p<0.05; ** p<0.01; *** p<0.001 as compared to the untreated CDKL5+/Y condition; #p<0.05 as compared to the untreated CDKL5 −/Y samples (Fisher LSD test after ANOVA).

FIG. 72 is a graph showing the number of fluorescent puncta per μm² exhibiting PSD-95 immunoreactivity in the primary visual cortex (V1) of untreated CDKL5+/Y (n=3) and CDKL5 −/Y (n=3) mice, and CDKL5 −/Y mice treated with TATκ-eGFP (n=4) or TATκ-eGFP-CDKL5₁₀₇ (n=6) and sacrificed at the end of treatment (short term), or with TATκ-eGFP (n=4) or TATκ-eGFP-CDKL5₁₀₇ (n=4) and sacrificed 10 days after treatment cessation (long term). Values are represented as means±SE. ** p<0.01 as compared to the untreated CDKL5+/Y condition; #p<0.05 as compared to the untreated CDKL5 −/Y samples (Fisher LSD test after ANOVA).

### DETAILED DESCRIPTION

Provided herein are TATκ-CDKL5 fusion protein compositions and formulations and methods for their use in the treatment of CDKL5-mediated disease and disorders, particularly disorders and diseases due to CDKL5 mutations and/or deficiencies. Also provided herein are methods for producing TATκ-CDKL5 fusion protein compositions and formulations. These methods provide for improved experimental tools for the research of CDKL5-mediated neurological disorders as well as improved treatment options for patients suffering disorders related to CDKL5 dysfunction. Also provided herein are methods of using such TATκ-CDKL5 fusion protein compositions and formulations for treating CDKL5-mediated diseases and disorders. Also provided herein are methods of increasing neural activity in the visual cortex in a patient having a CDKL5-mediated disease or disorder. Also provided herein are methods of increasing neurite growth, elongation, dendritic spine number, branch number, or branch density in a brain of a patient having a CDKL5-mediated disease or disorder. Also provided herein are methods of reducing neuronal apoptosis in the brain of a patient having a CDKL5-mediated disease or disorder. Also provided herein are methods of improving motor function of a patient having a CDKL5-mediated disease or disorder.

### Definitions

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “additive effect” refers to an effect arising between two or more molecules, compounds, substances, factors, or compositions that is equal to or the same as the sum of their individual effects.

The term “amphiphilic,” as used herein, refers to a molecule combining hydrophilic and lipophilic (hydrophobic) properties.

As used herein, “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen.

As used herein, “anti-infective” refers to compounds or molecules that can either kill an infectious agent or inhibit it from spreading. Anti-infectives include, but are not limited to, antibiotics, antibacterials, antifungals, antivirals, and antiprotozoans.

As used herein, “aptamer” refers to single-stranded DNA or RNA molecules that can bind to pre-selected targets including proteins with high affinity and specificity. Their specificity and characteristics are not directly determined by their primary sequence, but instead by their tertiary structure.

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

As used herein “biodegradable” generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “hydrophilic”, as used herein, refers to substances that have strongly polar groups that readily interact with water.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “CDKL5 deficiency” refers to any deficiency in the biological function of the protein. The deficiency can result from any DNA mutation in the DNA coding for the protein or a DNA related regulatory region or any change in the function of the protein due to any changes in epigenetic DNA modification, including but not limited to DNA methylation or histone modification, any change in the secondary, tertiary, or quaternary structure of the CDKL5 protein, or any change in the ability of the CDKL5 protein to carry out its biological function as compared to a wild-type or normal subject.

As used herein, “CDKL5 mutation” refers to any change in the nucleotide sequence of the coding region of the CDKL5 protein.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “composition” refers to a combination of active agent and at least one other compound or molecule, inert (for example, a detectable agent or label) or active, such as an adjuvant.

As used herein, “concentrated” refers to a molecule, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “control” is an alternative subject or sample used in an experiment for comparison purpose and included to minimize or distinguish the effect of variables other than an independent variable.

As used herein, “chemotherapeutic agent” or “chemotherapeutic” refer to a therapeutic agent utilized to prevent or treat cancer.

As used herein, “culturing” refers to maintaining cells under conditions in which they can proliferate and avoid senescence as a group of cells. “Culturing” can also include conditions in which the cells also or alternatively differentiate.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “DNA molecule” includes nucleic acids/polynucleotides that are made of DNA.

As used herein, “derivative” refers to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring with saturated or unsaturated cyclohexane or other more complex, e.g., nitrogen-containing rings, and extensions of these rings with side various groups.

As used herein, “differentiate” or “differentiation,” refers to the process by which precursor or progenitor cells (e.g., neuronal progenitor cells) differentiate into specific cell types (e.g., neurons).

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “diluted” refers to a molecule, including but not limited to a polynucleotide, peptide, polypeptide, protein, antibody, or fragments thereof, that is distinguishable from its naturally occurring counterpart in that the concentration or number of molecules per volume is less than that of its naturally occurring counterpart.

As used herein, “dose,” “unit dose,” or “dosage” refers to physically discrete units suitable for use in a subject, each unit containing a predetermined quantity of the CDKL5 fusion protein, a composition containing the CDKL5 fusion protein, and/or a pharmaceutical formulation thereof calculated to produce the desired response or responses in association with its administration.

As used herein, “effective amount” is an amount sufficient to effect beneficial or desired biological, emotional, medical, or clinical response of a cell, tissue, system, animal, or human. An effective amount can be administered in one or more administrations, applications, or dosages. The term also includes within its scope amounts effective to enhance normal physiological function.

As used herein, “expansion” or “expanded” in the context of cell refers to an increase in the number of a characteristic cell type, or cell types, from an initial population of cells, which may or may not be identical. The initial cells used for expansion need not be the same as the cells generated from expansion. For instance, the expanded cells may be produced by ex vivo or in vitro growth and differentiation of the initial population of cells.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “fusion protein” refers to a protein formed from the combination of at least two different proteins or protein fragments. A fusion protein is encoded by a recombinant DNA molecule. As such, a “CDKL5 fusion protein” refers to a recombinant protein having a human CDKL5 polypeptide or variant thereof operatively linked to other polypeptide sequences.

As used herein, “gene” refers to a hereditary unit corresponding to a sequence of DNA that occupies a specific location on a chromosome and that contains the genetic instruction for a characteristic(s) or trait(s) in an organism.

As used herein, “green fluorescent protein,” “yellow fluorescent protein,” “red fluorescent protein” and the like and their abbreviations include, without limitation, all forms of such proteins as they are routinely modified, derivatized, and generally known to those of ordinary skill in the art. For example “green fluorescent protein” includes, without limitation, enhanced green fluorescent protein (eGFP), redox sensitive GFP (roGFP), and all color mutants.

As used herein, “HEK 293T” and is a term of art to refer to human embryonic kidney (HEK) 293 cells that express large T antigen and are generally known in the art and commercially available through vendors such as American Tissue Type Culture Collection.

The term “hydrophobic”, as used herein, refers to substances that lack an affinity for water; tending to repel and not absorb water as well as not dissolve in or mix with water. As used herein, “identity,” is a relationship between two or more polypeptide sequences, as determined by comparing the sequences. In the art, “identity” also refers to the degree of sequence relatedness between polypeptide as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure. As used herein, “immunomodulator,” refers to an agent, such as a therapeutic agent, which is capable of modulating or regulating one or more immune function or response.

As used herein “induces,” “inducing,” or “induced” refers to activating or stimulating a process or pathway within a cell, such as endocytosis, secretion, and exocytosis.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

The term “lipophilic,” as used herein, refers to compounds having an affinity for lipids.

As used herein, “mammal,” for the purposes of treatments, refers to any animal classified as a mammal, including human, domestic and farm animals, nonhuman primates, and zoo, sports, or pet animals, such as, but not limited to, dogs, horses, cats, and cows.

As used herein, “matrix” refers to a material, in which one or more specialized structures, molecules, or compositions, are embedded.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. Also, polynucleotide, as used herein, refers to triple-stranded regions comprising RNA or DNA or both RNA and DNA. The strands in such regions may be from the same molecule or different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, among other things. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or other reasons are “nucleic acids” or “polynucleotide” as that term is intended herein.

As used herein, “nucleic acid sequence” and “oligonucleotide” also encompasses a nucleic acid and polynucleotide as defined above. As used herein, “organism,” “host,” and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). “Subject” may also be a cell, a population of cells, a tissue, an organ, or an organism, preferably to human and constituents thereof. As used herein, “overexpressed” or “overexpression” refers to an increased expression level of an RNA or protein product encoded by a gene as compared to the level of expression of the RNA or protein product in a normal or control cell.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector.

As used herein, “patient” refers to an organism, host, or subject in need of treatment.

As used herein “peptide” refers to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “plasmid” as used herein refers to a non-chromosomal double-stranded DNA sequence including an intact “replicon” such that the plasmid is replicated in a host cell.

As used herein, “positive control” refers to a “control” that is designed to produce the desired result, provided that all reagents are functioning properly and that the experiment is properly conducted.

As used herein, “preventative” and “prevent” refers to hindering or stopping a disease or condition before it occurs, even if undiagnosed, or while the disease or condition is still in the sub-clinical phase.

As used herein, “protein” as used herein refers to a large molecule composed of one or more chains of amino acids in a specific order. The term protein is used interchangeable with “polypeptide.” The order is determined by the base sequence of nucleotides in the gene coding for the protein. Proteins are required for the structure, function, and regulation of the body's cells, tissues, and organs. Each protein has a unique function.

As used herein, “purified” or “purify” is used in reference to a nucleic acid sequence, peptide, or polypeptide that has increased purity relative to the natural environment.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “Rett syndrome variant,” “variant of Rett syndrome,” and the like refers to an atypical form of Rett syndrome with similar clinical signs to Rett syndrome but an unknown etiology.

As used herein, “separated” refers to the state of being physically divided from the original source or population such that the separated compound, agent, particle, or molecule can no longer be considered part of the original source or population.

As used herein, “specifically binds” or “specific binding” refers to binding that occurs between such paired species such as enzyme/substrate, receptor/agonist or antagonist, antibody/antigen, lectin/carbohydrate, oligo DNA primers/DNA, enzyme or protein/DNA, and/or RNA molecule to other nucleic acid (DNA or RNA) or amino acid, which may be mediated by covalent or non-covalent interactions or a combination of covalent and non-covalent interactions. When the interaction of the two species produces a non-covalently bound complex, the binding that occurs is typically electrostatic, hydrogen-bonding, or the result of lipophilic interactions. Accordingly, “specific binding” occurs between a paired species where there is interaction between the two which produces a bound complex having the characteristics of an antibody/antigen, enzyme/substrate, DNA/DNA, DNA/RNA, DNA/protein, RNA/protein, RNA/amino acid, receptor/substrate interaction. In particular, the specific binding is characterized by the binding of one member of a pair to a particular species and to no other species within the family of compounds to which the corresponding member of the binding member belongs. Thus, for example, an antibody preferably binds to a single epitope and no other epitope within the family of proteins.

As used herein, “specific binding partner” or “binding partner” is a compound or molecule to which a second compound or molecule binds with a higher affinity than all other molecules or compounds.

As used interchangeably herein, “subject,” “individual,” or “patient” refers to a vertebrate organism.

As used herein, “substantially pure” means an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used herein, “substantially pure cell population” refers to a population of cells having a specified cell marker characteristic and differentiation potential that is about 50%, preferably about 75-80%, more preferably about 85-90%, and most preferably about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

The terms “sufficient” and “effective,” as used interchangeably herein, refer to an amount (e.g. mass, volume, dosage, concentration, and/or time period) needed to achieve one or more desired result(s). For example, a therapeutically effective amount refers to an amount needed to achieve one or more therapeutic effects.

As used herein, “synergistic effect,” “synergism,” or “synergy” refers to an effect arising between two or more molecules, compounds, substances, factors, or compositions that is greater than or different from the sum of their individual effects.

As used herein, “therapeutic” refers to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof. The disease or disorder can be a CDKL5 deficiency and/or Rett Syndrome.

As used herein, “therapeutically effective amount” refers to the amount of a CDKL5-fusion protein, a composition containing a CDKL5 fusion protein, a pharmaceutical formulation thereof, auxiliary agent, or secondary agent described herein that will elicit the biological or medical response of a tissue, system, animal, or human that is being sought by the researcher, veterinarian, medical doctor or other clinician. “Therapeutically effective amount” includes that amount of a CDKL5-fusion protein, a composition containing a CDKL5 fusion protein, a pharmaceutical formulation thereof that, when administered alone or co-administered with a secondary agent, is sufficient to prevent development of, reduce or alleviate to some extent, one or more of the symptoms of CDKL5 deficiency and/or Rett syndrome. “Therapeutically effect amount” includes that amount of CDKL5-fusion protein, a composition containing a CDKL5 fusion protein, a pharmaceutical formulation thereof that, when administered alone or co-administered with a secondary agent, is sufficient to increase neuron survival, neuron number, neurite growth, elongation, dendritic spine number, and/or branch density in a region of the brain of a subject as compared to a control. “Therapeutically effect amount” includes that amount of CDKL5-fusion protein, a composition containing a CDKL5 fusion protein, a pharmaceutical formulation thereof that, when administered alone or co-administered with a secondary agent, is sufficient to increase learning ability in a subject as compared to a control. “Therapeutically effect amount” includes that amount of CDKL5-fusion protein, a composition containing a CDKL5 fusion protein, a pharmaceutical formulation thereof that, when administered alone or co-administered with a secondary agent, is sufficient to increase memory ability in a subject as compared to a control. “Therapeutically effect amount” includes that amount of CDKL5-fusion protein, a composition containing a CDKL5 fusion protein, a pharmaceutical formulation thereof that, when administered alone or co-administered with a secondary agent, is sufficient to improve motor function in a subject as compared to a control. “Therapeutically effect amount” includes that amount of CDKL5-fusion protein, a composition containing a CDKL5 fusion protein, a pharmaceutical formulation thereof that, when administered alone or co-administered with a secondary agent, is sufficient to restore learning ability, memory ability, and/or motor function to levels that are substantially similar to wild-type or normal levels. “Therapeutically effect amount” includes that amount of CDKL5-fusion protein, a composition containing a CDKL5 fusion protein, a pharmaceutical formulation thereof that, when administered alone or co-administered with a secondary agent, is sufficient to restore neuron number, neuron survival, neurite growth, neurite elongation, dendritic spine number, neurite branch number, and/or neurite branch density in a region of the brain to levels that are substantially similar to wild-type or normal levels. The therapeutically effective amount will vary depending on the exact chemical structure of the CDKL5-fusion protein, a composition containing a CDKL5 fusion protein, a pharmaceutical formulation thereof, the CDKL5 deficiency, Rett syndrome or symptom thereof being treated, the route of administration, the time of administration, the rate of excretion, the drug combination, the judgment of the treating physician, the dosage form, and the age, weight, general health, sex and/or diet of the subject to be treated.

The terms “treating” and “treatment” as used herein refer generally to obtaining a desired pharmacological and/or physiological effect. The effect may be prophylactic in terms of preventing or partially preventing a disease, symptom or condition thereof, such as disease or disorders resulting from CDKL5 mutations and/or deficiencies, the CDKL5 variant of Rett syndrome, or other CDKL5-mediated neurological disorder, and/or may be therapeutic in terms of a partial or complete cure of a disease, condition, symptom or adverse effect attributed to the disease, disorder, or condition. The term “treatment” as used herein covers any treatment of CDKL5-mediated neurological disorder in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “treatment” as used herein refers to both therapeutic treatment and prophylactic or preventative measures. Those in need of treatment include those already with the disorder as well as those in which the disorder is to be prevented.

As used herein, “tangible medium of expression” refers to a medium that is physically tangible and is not a mere abstract thought or an unrecorded spoken word. Tangible medium of expression includes, but is not limited to, words on a cellulosic or plastic material or data stored on a suitable device such as a flash memory or CD-ROM.

As used herein, “transduced” refers to the direct introduction of a protein into a cell.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome. It may be incorporated into a viral particle.

As used herein, “transformation” or “transformed” refers to the introduction of a nucleic acid (e.g., DNA or RNA) into cells in such a way as to allow expression of the coding portions of the introduced nucleic acid.

As used herein, “underexpressed” or “underexpression” refers to decreased expression level of an RNA or protein product encoded by a gene as compared to the level of expression of the RNA or protein product in a normal or control cell.

As used herein, “variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. “Variant” includes functional and structural variants.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, “wild-type” is the typical form of an organism, variety, strain, gene, protein, or characteristic as it occurs in nature, as distinguished from mutant forms that may result from selective breeding or transformation with a transgene.

Unless otherwise defined herein, all technical and scientific terms used herein have the same meaning as commonly understood by one of ordinary skill in the art.

### Discussion

TATκ-CDKL5 Fusion Genes and Proteins

In human CDKL5 the first two reported splice variants differ in the 5′UTRs and produce the same 115 kDa protein. They are referred to as isoform I, containing exon 1, which is transcribed in a wide range of tissues, and isoform II, including exon 1a and 1b, which is limited to testis and fetal brain (Kalscher et al. 2003. Am J Hum Genet, 72: 1401-11 and Williamson et al. 2012. Hum Genet, 131: 187-200). The resulting CDKL5 transcript generates a protein of 1030 amino acids with molecular weight of 115 kDa (CDKL5₁₁₅) and is mainly expressed in the testis. More recently alternative splicing events have been identified, leading to at least three distinct human protein isoforms. One of these isoforms is, characterized by an altered C-terminal region and is referred to as CDKL5₁₀₇. (Fichou et al. 2011. J. Hum Genet, 56:52-57, Williamson et al. 2012, Hector et. al. 2016. PLoS One. 11(6): e0157758). CDKL5₁₀₇ is the predominant isoform in human and mouse brains. In all human tissues CDKL5₁₀₇ is the most abundant transcript, with tissues expressing 10- to 100-fold or more CDKL5₁₀₇ than CDKL5₁₁₅. In particular, in the whole brain, there is 37-fold more of CDKL5₁₀₇ than CDKL5₁₁₅. Testis is the exception, with only 2.5-fold more CDKL5₁₀₇ than CDKL5₁₁₅ reflecting the relative abundance of CDKL5₁₁₅ in this tissue. The C-terminus of CDKL5, which is different in the two isoforms, is important in modulating its subcellular localization, and the accumulation of truncated protein in the nucleus. (Bertani et al. 2006. J Biol Chem, 281:32048-56 and Rusconi et al. 2008. J Biol Chem, 283:30101-11).

The cellular distribution of the CDKL5₁₀₇ isoform has been examined, revealing subcellular localization and catalytic activity that overlap greatly, but not completely, with that of the previously studied human CDKL5₁₁₅ protein. In vitro data indicate that proteasomal degradation of the CDKL5₁₁₅ isoform is mediated by a signal between amino acids 904 and 1030, exclusively present in this isoform, whereas CDKL5₁₀₇ is more stable and less prone to degradation through the proteasome pathway.

Fusion Genes and Proteins

Disclosed herein are recombinant cDNA sequences, which code for various CDKL5 fusion proteins containing a modified TAT (TATκ) sequence. The CDKL5 included in the fusion protein can be the CDKL5₁₁₅ isoform. The CDKL5 included in the fusion protein can be the CDKL5₁₀₇ isoform. SEQ ID NO: 2 corresponds to the CDKL5₁₁₅ isoform polypeptide and SEQ ID NO: 16 corresponds to the CDKL5₁₀₇ isoform polypeptide. In one embodiment the fusion protein contains a human CDKL5 polypeptide operatively coupled to a TATκ polypeptide. The cDNA sequence, which codes for the CDKL5 fusion protein, can have a sequence according to any one of SEQ ID NOs: 1, 7, 9, 11, 13, or a variant thereof described herein. The CDKL5 fusion protein can have a polypeptide sequence according to any one of SEQ ID NOs: 8, 10, 12, 14, or a variant thereof describe herein.

In some embodiments, the human CDKL5 cDNA sequence can be according to SEQ ID NOs: 1 or 15. In further embodiments, the human CDKL5 cDNA can be about 90% to about 100%, 80% to about 90%, or about 50% to about 80%, identical to SEQ ID NOs: 1 or 15. In some embodiments, the human CDKL5 cDNA sequence can code for an amino acid sequence according to SEQ ID NO: 2 or 16. In further embodiments, the human CDKL5 cDNA sequence can code for an amino acid sequence that is about 90% to about 100%, 80% to about 90%, or about 50% to about 80% identical to SEQ ID NO: 2 or 16.

In some embodiments, the human CDKL5 cDNA sequence can be a fragment of at least 12 consecutive nucleotides that are about 90% to 100% identical to 12 consecutive nucleotides in SEQ ID NO: 1. In some embodiments, the human CDKL5 cDNA sequence can be a fragment of at least 12 consecutive nucleotides that are about 80% to 90% identical to 12 consecutive nucleotides in SEQ ID NO: 1. In some embodiments, the cDNA sequence can be a fragment of at least 12 consecutive nucleotides that are about 50% to 80% identical to 12 consecutive nucleotides in SEQ ID NO: 1.

In some embodiments, the human CDKL5 cDNA sequence can be a fragment of at least 12 consecutive nucleotides that are about 90% to 100% identical to 12 consecutive nucleotides in SEQ ID NO: 15. In some embodiments, the human CDKL5 cDNA sequence can be a fragment of at least 12 consecutive nucleotides that are about 80% to 90% identical to 12 consecutive nucleotides in SEQ ID NO: 15. In some embodiments, the cDNA sequence can be a fragment of at least 12 consecutive nucleotides that are about 50% to 80% identical to 12 consecutive nucleotides in SEQ ID NO: 15.

The CDKL5 fusion protein contains a modified trans-acting activation of transcription (TAT) protein transduction domain (PTD) (TATκ) operatively coupled to the human CDKL5 polypeptide. The TATκ can have a cDNA sequence according to SEQ ID NO: 3 and an amino acid sequence according to SEQ ID NO: 4. TATκ is a modified TAT-PTD. Unmodified TAT-PTD mediates the transductions of peptides and proteins into cells. However, unmodified TAT-PTD does not allow TAT-PTD fusion proteins to be secreted by the cell. Unmodified TAT-PTD is cleaved from the fusion protein by furin endoprotease at furin recognition sequences located within unmodified TAT-PTD. In contrast, TATκ is modified such that it does not contain the furin recognition sequences. As such, the CDKL5 fusion proteins described herein containing TATκ can be secreted in its full form by eukaryotic cells.

In some embodiments, the TATκ cDNA sequence can be about 90% to 100% or about 80% to about 90% identical to SEQ ID NO: 3. In some embodiments, the TATκ cDNA can code for a polypeptide sequence that is about 90% to 100% or about 80% to about 90% identical to SEQ ID NO: 4. The TATκ cDNA can be operatively coupled to a cDNA that encodes a CDKL5 fusion protein.

The CDKL5 fusion protein can optionally contain an Igκ-chain leader sequence to direct the polypeptide down the secretory pathway during production by a cell. In some embodiments, the Igκ-chain leader sequence can be operatively coupled at the N-terminus of the human CDKL5 polypeptide. The Igκ-chain leader sequence can have a cDNA sequence according to SEQ ID NO: 5 or a variant thereof described herein and can have an amino acid sequence according to SEQ ID NO: 6 or variant thereof described herein. The Igκ-chain leader sequence cDNA can be operatively coupled to a cDNA that encodes a CDKL5 fusion protein.

In other embodiments, the Igκ-chain leader sequence cDNA can be about 90% to 100%, about 80% to about 90%, or about 80% to 90% identical to SEQ ID NO: 5. In some embodiments, the Igκ-chain leader sequence can have an amino acid sequence that is about 90% to about 100%, about 80% to about 90%, or about 50% to about 80% identical to SEQ ID NO: 6.

The CDKL5 fusion protein can optionally contain one or more protein tags operatively coupled to the CDKL5 fusion protein. These types of tags are amino acid sequences that allow for affinity purification, solubilization, chromatographical separation, and/or immunodetection of the fusion protein. Suitable protein tags include, but are not limited to, chitin binding protein (CBP), maltose binding protein (MBP), glutathione-S-transferase (GST), poly(His), thioredoxin (TRX), poly(NANP), FLAG-tag (including any FLAG-tag variant, e.g. 3×FLAG), V5-tag, Myc-tag, HA-tag, S-tag, SBP-Tag, Sftag 1, Softag 3, Tc tag, Xpress tag, Strep-tag, Isopeptag, Spy Tag, Ty tag, Biotin Carboxyl Carrier Protein (BCCP), and Nus tag. A CDKL5 fusion protein cDNA according SEQ ID NO: 7, 9, or 11 having an amino acid sequence according to SEQ ID NO: 8, 10, or 12, respectively, demonstrate non-limiting embodiments of a CDKL5 fusion protein containing a TATκ, and Myc-tag and a poly(HIS) tag. A CDKL5 fusion protein cDNA according SEQ ID NO: 13, having an amino acid sequence according to SEQ ID NO: 14 demonstrate a non-limiting embodiment of a CDKL5 fusion protein having a FLAG-tag.

The CDKL5 fusion protein can optionally contain one or more reporter proteins operatively coupled to the CDKL5 polypeptide. Suitable reporter genes include, but are not limited to, fluorescent proteins (e.g. green fluorescent protein (GFP), red fluorescent protein (RFP), yellow fluorescent protein (YFP), blue fluorescent protein (BFP), and cyan fluorescent protein (CFP)), beta-galactosidase, luciferase (bacterial, firefly, and renilla luciferase), antibiotic-resistance genes (e.g. chloramphenicol acetyltransferase, neomycin phosphotransferase, and NPT-II), p-glucuronidase, and alkaline phosphatase. Inclusion of a reporter protein allows, among other things, for direct and/or indirect characterization of the fusion protein and function of the fusion protein, as well as affinity purification of the protein. The reporter protein can be operatively linked to the N-terminus and/or the C-terminus of the human CDKL5 polypeptide. In other embodiments, the reporter protein can be operatively linked to N-terminus and/or the C-terminus of the CDKL5 fusion protein. A CDKL5 fusion protein cDNA according SEQ ID NO: 9 or 11 and having an amino acid sequence according to SEQ ID NO: 10 or 12 respectively, demonstrate non-limiting embodiments of a CDKL5 fusion protein containing a fluorescent reporter protein.

Recombinant Vectors

The CDKL5 fusion cDNA sequence can be incorporated into a suitable expression vector. The expression vector can contain one or more regulatory sequences or one or more other sequences used to facilitate the expression of the CDKL5 fusion cDNA. The expression vector can contain one or more regulatory sequences or one or more other sequences used to facilitate the replication of the CDKL5 fusion expression vector. The expression vector can be suitable for expressing the CDKL5 fusion protein in a bacterial cell. In other embodiments, the expression vector can be suitable for expressing the CDKL5 fusion protein in a yeast cell. In further embodiments, the expression vector can be suitable for expressing the CDKL5 fusion protein in a plant cell. In other embodiments, the expression vector can be suitable for expressing the CDKL5 fusion protein in a mammalian cell. In another embodiment, the vector can be suitable for expressing the CDKL5 fusion protein in a fungal cell. In further embodiments, the vector can be suitable for expressing the CDKL5 fusion protein in an insect cell. Suitable expression vectors are generally known to those of ordinary skill in the art.

TATκ-CDKL5 Protein Production

In some embodiments, the CDKL5 fusion protein is produced in vitro in a cell culture system. The cell culture system can contain one or more bacterial, yeast, insect, fungal, plant, or mammalian cells. In some embodiments, the CDKL5 fusion protein is secreted by the cultured cell(s) into the cell culture media. In other embodiments, the CDKL5 fusion protein is contained within the cytoplasm or a membrane of the cultured cell(s).

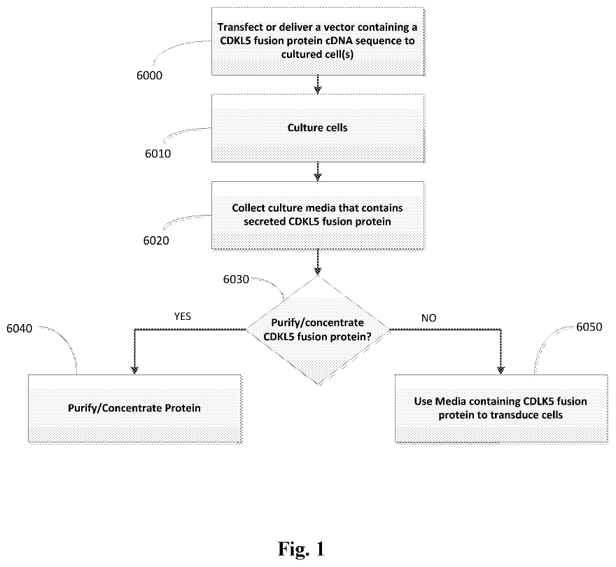

With that said, attention is directed to FIG. 1, which shows one embodiment of a method to produce a CDKL5 fusion protein, wherein the CDKL5 fusion protein is produced by the cultured cell and secreted into the surrounding culture medium. The method begins by transfecting or otherwise delivering a suitable vector containing a CDKL5 fusion protein cDNA sequence to a cell or cells in culture (6000). The cells are then cultured (6010) using generally known methods to allow the transfected cells to produce the CDKL5 fusion protein from the vector and secrete the CDKL5 fusion protein into the surrounding cell culture medium. In other embodiments, stably-transfected cell lines expressing one or more of the vectors containing a CDKL5 fusion protein cDNA can be generated using techniques generally known by those of ordinary skill in the art. These cells can be cultured (6010) using generally know methods to allow the cells to produce the CDKL5 fusion protein.

After a suitable amount of time, the culture medium that contains the secreted CDKL5 fusion protein is collected (6020). In some embodiments the cells are cultured from about 12 h to about 96 h. At this point, it is determined whether or not the CDKL5 fusion protein needs to be further purified from the culture medium (6030). In some embodiments, the medium containing the CDKL5 fusion protein is not further purified and is used directly to transduce one or more cells (6050). In other embodiments, the CDKL5 fusion protein is further purified from and/or concentrated in the culture media. In some embodiments, the CDKL5 fusion protein is purified and/or concentrated using a suitable method. Suitable methods include, but are not limited to solvent partitioning salting in or salting out, affinity-based methods, and chromatographic separation methods such as hydrophobic interaction, ion exchange, mixed mode, dye binding, and size exclusion, as exemplified in Kameshita et al. (Biochemical and Biophysical Research Communications 377, 1162-1167 (2008)), Sekiguchi et al. (Archives of Biochemistry and Biophysics 535, 257-267 (2013)), and Katayama et al. (Biochemistry, 54, 2975-2987 (2015)), which are incorporated by reference.

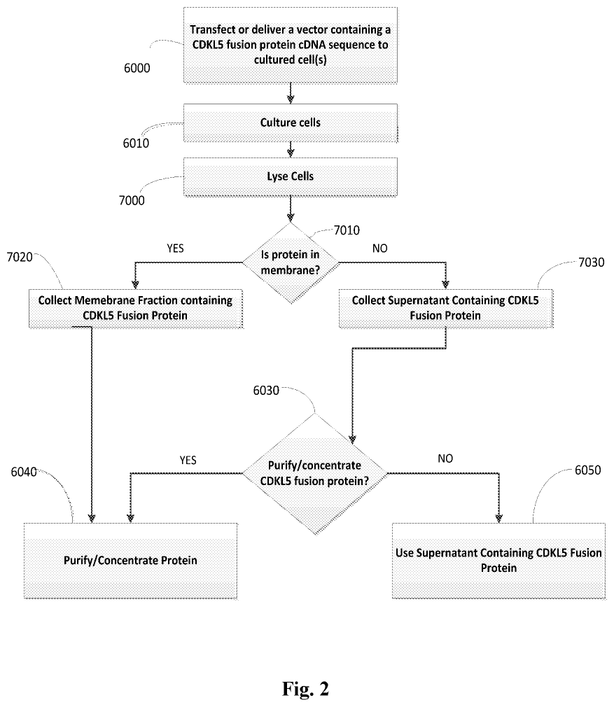

With an understanding of a secretory method of production in mind, attention is directed to FIG. 2, which shows one embodiment of a method of producing a CDKL5 fusion protein wherein the CDKL5 fusion protein is not secreted into the surrounding cell culture media. The method begins by transfecting or otherwise delivering a suitable vector containing a CDKL5 fusion protein cDNA sequence to a cell or cells in culture (6000). The cells are then cultured (6010) using generally known methods to allow the transfected cells to produce the CDKL5 fusion protein from the vector. After a suitable amount of time, the cells are lysed using standard methods (7000). In some embodiments, the cells are cultured from 12 h to 96 h before being lysed.

Next, it is determined if the CDKL5 fusion protein is integrated within the cell membrane or the cytoplasm (7010). If the CDKL5 fusion protein is in the membrane fraction, then the membrane fraction is collected (7020). After the membrane fraction is collected (7020), the CDKL5 fusion protein is separated from the membrane fraction using suitable method (6040) for purifying and/or concentrating the CDKL5 fusion protein.

In embodiments where the CDKL5 fusion protein is present in the cytoplasm, the supernatant containing the CDKL5 fusion protein is collected (7030). After the supernatant is collected (7030), it is determined if the CDKL5 fusion protein should be further purified and/or concentrated. If it is determined that that the CDKL5 fusion protein should be further purified and/or concentrated, then the CDKL5 fusion protein is purified and/or concentrated using a suitable method (6040). Suitable methods include, but are not limited to, affinity purification, size exclusion separation, and chromatographical separation methods. In other embodiments where it is determined that the CDKL5 should not be further purified and/or concentrated from the supernatant, the supernatant containing the CDKL5 fusion protein is used directly to transduce cells (6050).

Compositions and Formulations Containing TATκ-CDKL5 Fusion Protein

Also within the scope of this disclosure are compositions and formulations containing a CDKL5 fusion protein as described herein. The composition can be the media or supernatant containing the CDKL5 fusion protein that can be produced according to a method described herein.

The CDKL5 fusion proteins described herein can be provided to a subject in need thereof alone or as such as an active ingredient, in a pharmaceutical formulation. As such, also described herein are pharmaceutical formulations containing an amount of a CDKL5 fusion protein. In some embodiments, the pharmaceutical formulations contain a therapeutically effective amount of a CDKL5 fusion protein. The pharmaceutical formulations described herein can be administered to a subject in need thereof. The subject in need thereof can have a CDKL5 deficiency, Rett syndrome, and/or a symptom thereof. In other embodiments, the CDKL5 fusion protein can be used in the manufacture of a medicament for the treatment or prevention of a CDKL5 deficiency, Rett syndrome, and/or a symptom thereof.

Pharmaceutically Acceptable Carriers and Auxiliary Ingredients and Agents

The pharmaceutical formulations containing a therapeutically effective amount of a CDKL5 fusion protein described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to the therapeutically effective amount of a of a CDKL5 fusion protein described herein, the pharmaceutical formulation can also include an effective amount of an auxiliary active agent, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e g aminosalicylates (e g aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid

Effective Amounts of the CDKL5 Fusion Protein and Auxiliary Agents

The pharmaceutical formulations can contain a therapeutically effective amount of a CDKL5 fusion protein, and optionally, a therapeutically effective amount of an auxiliary agent. The precise dosage will vary with the age, size, sex and condition of the subject, the nature and severity of the disorder to be treated, and the like; thus, a precise effective amount cannot be specified in advance and will be determined by the caregiver. However, the therapeutically effective amount of the CDKL5 fusion protein can generally range from about 1 μg/kg to about 10 mg/kg. In further embodiments, the therapeutically effective amount of the CDKL5 fusion protein can range from 1 ng/g bodyweight to about 0.1 mg/g bodyweight. The therapeutically effective amount of the CDKL5 fusion protein can range from about 1 pg to about 10 g. In some embodiments, the therapeutically effective amount of the CDKL5 fusion protein or pharmaceutical composition containing the CDKL5 fusion protein can range from about 10 nL to about 10 mL. In other embodiments the therapeutically effective amount of the CDKL5 fusion protein or pharmaceutical composition from about 10 nL to about 1 μL.

In some embodiments, the therapeutically effective amount can also range from about 20 to about 50 ng per injection, such as for an intraventricular injection. In other embodiments, the therapeutically effective amount can be about 10 microliters per injection, such as for intraventricular injection. In further embodiments, the therapeutically effective amount can be about 5 ng/μL, such as for intraventricular injection. In yet further embodiments, the therapeutically effective amount can be about 1.9 μg/kg of bodyweight for intraventricular injection. In other embodiments, the therapeutically effective amount can be from about 1 to 3 μg/kg of bodyweight for intraventricular injection.

In other embodiments, the therapeutically effective amount can be from about 1 to about 2 micrograms per injection, such as for a systemically administered injection. In some embodiments, the therapeutically effective amount can be about 5 ng/μL, such as for systemic injections. For some embodiments, the therapeutically effective amount can be about 1 to about 1.5 μg per 5 g of bodyweight.

In one or more embodiments, the systemic administration is intravenous administration. The intravenous formulation can be administered by direct intravenous injection (i.v. bolus) or can be administered by infusion by addition to an appropriate infusion solution such as 0.9% sodium chloride injection or other compatible infusion solution. The most commonly used IV infusion system consists of a bag filled with IV fluids, a drip chamber, roller clamp (variable resistance controller) for control of the flow and tubing connected to an IV catheter. The elevated IV bag in this system serves as a pressure source, the roller clamp as a user-controlled resistor, and the IV catheter as a fixed resistor.

Most commonly, the rate of IV fluids flow is determined by the rate at which drops of liquid are observed falling through a drip chamber. Gravity infusion of the parenteral solution is accomplished by suspending the solution container several feet above the patient and connecting the solution container to the venopuncture site via a disposable intravenous administration set which includes a drip chamber and flexible delivery tube.

In embodiments where there is an auxiliary active agent contained in the pharmaceutical formulation in addition to the CDKL5 fusion protein, the therapeutically effective amount of the auxiliary active agent will vary depending on the auxiliary active agent. In some embodiments, the effective amount of the auxiliary active agent ranges from 0.001 micrograms to about 1 milligram. In other embodiments, the effective amount of the auxiliary active agent ranges from about 0.01 IU to about 1000 IU. In further embodiments, the effective amount of the auxiliary active agent ranges from 0.001 mL to about 1 mL. In yet other embodiments, the effective amount of the auxiliary active agent ranges from about 1% w/w to about 50% w/w of the total pharmaceutical formulation. In additional embodiments, the effective amount of the auxiliary active agent ranges from about 1% v/v to about 50% v/v of the total pharmaceutical formulation. In still other embodiments, the effective amount of the auxiliary active agent ranges from about 1% w/v to about 50% w/v of the total pharmaceutical formulation.

Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravireal, intracerebral, and intracerebroventricular and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the CDKL5 fusion protein or composition containing the CDKL5 fusion protein The oral dosage form can be administered to a subject in need thereof.

Where appropriate, the dosage forms described herein can be microencapsulated. The dosage form can also be prepared to prolong or sustain the release of any ingredient. In some embodiments, the CDKL5 fusion protein is the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water soluble polymer, water insoluble polymers, and/or pH dependent polymers, with or without water insoluble/water soluble non polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the CDKL5 fusion protein, auxiliary active ingredient, and/or pharmaceutically acceptable salt thereof can be formulated with a paraffinic or water-miscible ointment base. In other embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the CDKL5 fusion protein, the composition containing a CDKL5 fusion protein, auxiliary active ingredient, and/or pharmaceutically acceptable salt thereof in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient, which may be generated by various types of metered dose pressurized aerosols, nebulizers, or insufflators.

In some embodiments, the dosage forms are aerosol formulations suitable for administration by inhalation. In some of these embodiments, the aerosol formulation contains a solution or fine suspension of the CDKL5 fusion protein, the composition containing a CDKL5 fusion protein, and/or pharmaceutically acceptable salt thereof, and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a CDKL5 fusion protein, composition containing a CDKL5 fusion protein, or a pharmaceutical formulation thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the CDKL5 fusion protein, the composition containing a CDKL5 fusion protein, an auxiliary active ingredient, and/or pharmaceutically acceptable salt thereof, such a dosage form can contain a powder base such as lactose, glucose, trehalose, mannitol, and/or starch. In some of these embodiments, the CDKL5 fusion protein, the composition containing a CDKL5 fusion protein, auxiliary active ingredient, and/or pharmaceutically acceptable salt thereof is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol formulations are arranged so that each metered dose of aerosol contains a predetermined amount of an active ingredient, such as the one or more of the CDKL5 fusion proteins or compositions containing the CDKL5 fusion protein described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or non-aqueous sterile solutions that can optionally be adapted for injection, and which can optionally contain anti-oxidants, buffers, bacteriostats, solutes that render the composition isotonic with the eye or fluid contained therein or around the eye of the subject, and aqueous and non-aqueous sterile suspensions, which can include suspending agents and thickening agents.

For some embodiments, the dosage form contains a predetermined amount of the CDKL5 fusion protein or composition containing a CDKL5 fusion protein per unit dose. In an embodiment, the predetermined amount of the CDKL5 fusion protein or composition containing a CDKL5 fusion protein is a therapeutically effective amount of the CDKL5 fusion protein or composition containing a CDKL5 fusion protein to treat or prevent a CDKL5 deficiency, Rett syndrome, and/or a symptom thereof. In other embodiments, the predetermined amount of the CDKL5 fusion protein or composition containing a CDKL5 fusion protein can be an appropriate fraction of the therapeutically effective amount of the active ingredient. Such unit doses may, therefore, be administered once or more than once a day. Such pharmaceutical formulations may be prepared by any of the methods well known in the art.

Treatment of Neurological Disorders with TATκ-CDKL5 Compositions and Formulations

The CDKL5 fusion protein and pharmaceutical formulations thereof described herein can be used for the treatment and/or prevention of a disease, disorder, syndrome, or a symptom thereof in a subject. In some embodiments, the CDKL5 fusion protein and pharmaceutical formulations thereof can be used to treat and/or prevent a CDKL5 deficiency, Rett syndrome, variants of Rett syndrome, and/or a symptom thereof. In some embodiments, the subject has a CDKL5 deficiency, Rett syndrome, variants of Rett syndrome, and/or a symptom thereof. In some embodiments, the CDKL5 fusion protein and pharmaceutical formulations thereof can be used to increase neural activity in the visual cortex in a patient having a CDKL5 deficiency, Rett syndrome, variants of Rett syndrome, and/or a symptom thereof. In some embodiments, the CDKL5 fusion protein and pharmaceutical formulations thereof can be used to increase neurite growth, elongation, dendritic spine number, branch number, or branch density in a brain of a patient having a CDKL5 deficiency, Rett syndrome, variants of Rett syndrome, and/or a symptom thereof. In some embodiments, the CDKL5 fusion protein and pharmaceutical formulations thereof can be used to reduce neuronal apoptosis in the brain of a patient having a CDKL5 deficiency, Rett syndrome, variants of Rett syndrome, and/or a symptom thereof. In some embodiments, the CDKL5 fusion protein and pharmaceutical formulations thereof can be used to improve motor function of a patient having a CDKL5 deficiency, Rett syndrome, variants of Rett syndrome, and/or a symptom thereof.

An amount of the CDKL5 fusion protein, compositions, and pharmaceutical formulations thereof described herein can be administered to a subject in need thereof one or more times per day, week, month, or year. In some embodiments, the amount administered can be the therapeutically effective amount of the CDKL5 fusion protein, compositions, and pharmaceutical formulations thereof. For example, the CDKL5 fusion protein, compositions, and pharmaceutical formulations thereof can be administered in a daily dose. This amount may be given in a single dose per day. In other embodiments, the daily dose may be administered over multiple doses per day, in which each containing a fraction of the total daily dose to be administered (sub-doses). In some embodiments, the amount of doses delivered per day is 2, 3, 4, 5, or 6. In further embodiments, the compounds, formulations, or salts thereof are administered one or more times per week, such as 1, 2, 3, 4, 5, or 6 times per week. In other embodiments, the CDKL5 fusion protein, compositions, and pharmaceutical formulations thereof can be administered one or more times per month, such as 1 to 5 times per month. In still further embodiments, the CDKL5 fusion protein, compositions, and pharmaceutical formulations thereof can be administered one or more times per year, such as 1 to 11 times per year.

The CDKL5 fusion proteins, compositions, and pharmaceutical formulations thereof can be co-administered with a secondary agent by any convenient route. The secondary agent is a separate compound and/or formulation from the CDKL5 fusion proteins, compositions, and pharmaceutical formulations thereof. The secondary agent can be administered simultaneously with the CDKL5 fusion proteins, compositions, and pharmaceutical formulations thereof. The secondary agent can be administered sequentially with the CDKL5 fusion proteins, compositions, and pharmaceutical formulations thereof. The secondary agent can have an additive or synergistic effect to the CDKL5 fusion proteins, compositions, and pharmaceutical formulations thereof. Suitable secondary agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. In some embodiments the secondary agent is DCA.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e g aminosalicylates (e g aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In embodiments where the CDKL5 fusion proteins, compositions, and pharmaceutical formulations thereof are simultaneously co-administered with a secondary agent, the CDKL5 fusion proteins, compositions, and pharmaceutical formulations thereof can be administered to the subject at substantially the same time as the secondary agent. As used in this context “substantially the same time” refers to administration of the CDKL5 fusion proteins, compositions, and pharmaceutical formulations thereof and a secondary agent where the period of time between administration of the CDKL5 fusion protein, composition, or pharmaceutical formulation thereof and the secondary agent is between 0 and 10 minutes.

In embodiments where the CDKL5 fusion protein, composition, or pharmaceutical formulations thereof is sequentially co-administered with a secondary agent, the CDKL5 fusion protein, composition, or pharmaceutical formulations thereof can be administered first, and followed by administration of the secondary agent after a period of time. In other embodiments where the CDKL5 fusion protein, composition, or pharmaceutical formulations thereof is sequentially co-administered with a secondary agent, the secondary agent can be administered first, and followed by administration of the CDKL5 fusion protein, composition, or pharmaceutical formulations thereof after a period of time. In any embodiment, the period of time between administration of the CDKL5 fusion protein, composition, or pharmaceutical formulations thereof and the secondary agent can range from 10 minutes to about 96 hours. In some embodiments, the period of time can be about 10 minutes, about 30 minutes, about 1 hour, about 2 hours, about 4 hours, about 6 hours, about 8 hours, about 10 hours, or about 12 hours. The sequential administration can be repeated as necessary over the course of the period of treatment.

The amount of the CDKL5 fusion proteins, compositions, pharmaceutical formulations thereof that can be administered are described elsewhere herein. The amount of the secondary agent will vary depending on the secondary agent. The amount of the secondary agent can be a therapeutically effective amount. In some embodiments, the effective amount of the secondary agent ranges from 0.001 micrograms to about 1 milligram. In other embodiments, the amount of the secondary agent ranges from about 0.01 IU to about 1000 IU. In further embodiments, the amount of the secondary agent ranges from 0.001 mL to about 1 mL. In yet other embodiments, the amount of the secondary agent ranges from about 1% w/w to about 50% w/w of the total pharmaceutical formulation. In additional embodiments, the amount of the secondary agent ranges from about 1% v/v to about 50% v/v of the total pharmaceutical formulation. In still other embodiments, the amount of the secondary agent ranges from about 1% w/v to about 50% w/v of the total secondary agent composition or pharmaceutical formulation.

In some embodiments, the composition or formulation containing the CDKL5 fusion protein is administered to a patient via and injection. Suitable methods of injection include, but are not limited to, intravenous, intraperitoneal, subcutaneous, intramuscular, intradermal, intraosseous, epidural, intracardiac, intraarticular, intracavernous, intrathecal, intravireal, intracerebral, and intracerebroventricular injection Other suitable methods of administration of the composition or formulation containing the CDKL5 fusion protein include, but are not limited to, topical, transdermal, nasal, or oral delivery. In some embodiments, the dosage of the CDKL5 fusion protein ranges from about 0.01 μg/g bodyweight to about 10 mg/g bodyweight.

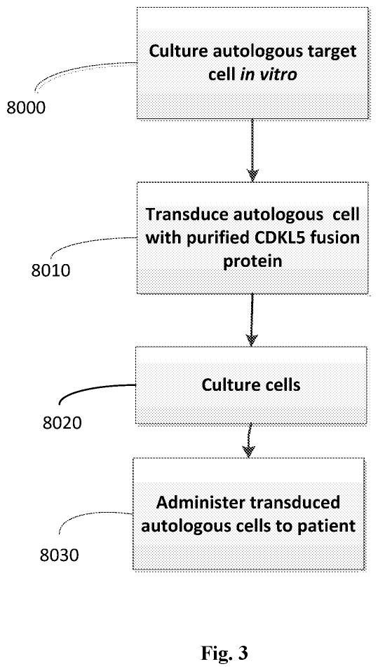

In other embodiments, the CDKL5 fusion protein can be delivered to a patient in need of treatment via cell therapy. With this in mind, attention is directed to FIG. 3, which shows one embodiment of method of delivering a CDKL5 fusion protein via an autologous cell. The method begins by culturing cells in vitro (8000). Preferably, the cells are autologous cells. In one embodiment, the autologous cells are neurons or neuronal precursor cells, such as neural stem cells. In some embodiments, the autologous cells are neurons that are derived from induced pluripotent stem cells. In other embodiments, the autologous cells are neurons that are derived from umbilical cord blood stem cells.

Next, the cultured cells are transduced with a purified CDKL5 fusion protein (8010). In other embodiments, the cultured cells are transduced by exposing the culture cells to media containing a CDKL5 fusion protein as previously described. In further embodiments, the cultured cells are transfected with a suitable vector containing a CDKL5 fusion protein cDNA. The cells are then cultured for a suitable amount of time to allow for expression of the CDKL5 fusion protein (8020). In some embodiments, the cells are cultured for about 6 h to about 96 h. After the cells are cultured, one or more transduced cells are administered to a patient.

In one embodiment, transduced autologous neurons are delivered to the brain using surgical techniques. In some embodiments, one or more transduced cells are administered to a patient via injection. In some embodiments, one or more transduced cells are included in a formulation. In one embodiment, the formulation containing one or more transduced cells also includes a pharmaceutically acceptable carrier and/or an active agent. In some embodiments the formulation containing the one or more transduced cells is administered to a patient via injection or using a surgical technique.

Kits Containing the CDKL5 Fusion Protein and Formulations Thereof

The CDKL5 fusion protein, compositions containing the CDKL5 fusion protein, and pharmaceutical formulations thereof described herein can be presented as a combination kit. As used herein, the terms “combination kit” or “kit of parts” refers to the CDKL5 fusion protein, compositions containing the CDKL5 fusion protein, and pharmaceutical formulations thereof described herein and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

The combination kit can contain each agent, compound, pharmaceutical formulation or component thereof, in separate compositions or pharmaceutical formulations. The separate compositions or pharmaceutical formulations can be contained in a single package or in separate packages within the kit. Also provided in some embodiments, are buffers, diluents, solubilization reagents, cell culture media and other reagents. These additional components can be contained in a single package or in separate packages within the kit.

In some embodiments, the combination kit also includes instructions printed on or otherwise contained in a tangible medium of expression. The instructions can provide information regarding the content of the CDKL5 fusion protein, compositions containing the CDKL5 fusion protein, and pharmaceutical formulations thereof and/or other auxiliary and/or secondary agent contained therein, safety information regarding the content of the CDKL5 fusion protein, compositions containing the CDKL5 fusion protein, and pharmaceutical formulations thereof and/or other auxiliary and/or secondary agent contained therein, information regarding the dosages, indications for use, and/or recommended treatment regimen(s) for the CDKL5 fusion protein, compositions containing the CDKL5 fusion protein, and pharmaceutical formulations thereof and/or other auxiliary and/or secondary agent contained therein. In some embodiments, the instructions can provide directions for administering the CDKL5 fusion protein, compositions containing the CDKL5 fusion protein, and pharmaceutical formulations thereof and/or other auxiliary and/or secondary agent to a subject having a CDKL5 deficiency, Rett syndrome, and/or a symptom thereof.

Without further elaboration, it is believed that one skilled in the art can, based on the description herein, utilize the present disclosure to its fullest extent. It is emphasized that the embodiments of the present disclosure, particularly any “preferred” embodiments, are merely possible examples of the implementations, merely set forth for a clear understanding of the principles of the disclosure. Many variations and modifications may be made to the disclosed embodiment(s) of the disclosure without departing substantially from the spirit and principles of the disclosure. All such modifications and variations are within the scope of this disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### EXAMPLES

The following examples are put forth so as to provide those of ordinary skill in the art with a complete disclosure and description of how to perform the methods and use the compositions and compounds disclosed and claimed herein. The specific examples below are to be construed as merely illustrative, and not limitative of the remainder of the disclosure in any way whatsoever. Efforts have been made to ensure accuracy with respect to numbers (e.g. amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1: Production and Purification of the TATκ-CDKL5₁₁₅ and TATκ-CDKL5₁₀₇ Fusion Proteins

To produce a deliverable TAT-CDKL5 fusion protein a synthetic TATκ-PTD in which mutation of the furin recognition sequences in the TAT domain allows secretion of recombinant proteins was used. The secreted protein was observed to be successfully taken up by the target cells. TATκ-CDKL5₁₁₅ or TATκ-CDKL5₁₀₇ fusion gene containing a human CDKL5₁₁₅ or CDKL5₁₀₇ was cloned into the expression plasmid pSecTag2 (Life Technologies). This plasmid is designed to allow expression of genes in mammalian hosts and high expression levels of target proteins. Proteins expressed from pSecTag2 are fused at the N-terminus to the murine Igκ chain leader sequence for protein secretion in culture medium. The TATκ-CDKL5 fusion proteins were tagged with an eGFP protein to allow for western blot analysis using an anti-GFP antibody. To facilitate protein purification, the TATκ-CDKL5 fusion proteins were configured to include a myc-tag, 6×His tag, and/or a FLAG tag at the C-terminal region of the TATκ-eGFP-CDKL5₁₁₅ or TATκ-eGFP-CDKL5₁₀₇ gene. HEK 293T cells were transfected with the TATκ-eGFP-CDKL5₁₁₅ or TATκ-eGFP-CDKL5₁₀₇ expression plasmid using standard plasmid delivery methods. After transfection cells were left to grow in serum-free medium (High glucose Dulbecco's Modified Eagle Medium). After 48 hours medium was collected, diafiltered and concentrated with Amicon ultra centrifugal filters (50 kDa cut-off). This method allows buffer exchange and enrichment of the secreted protein.

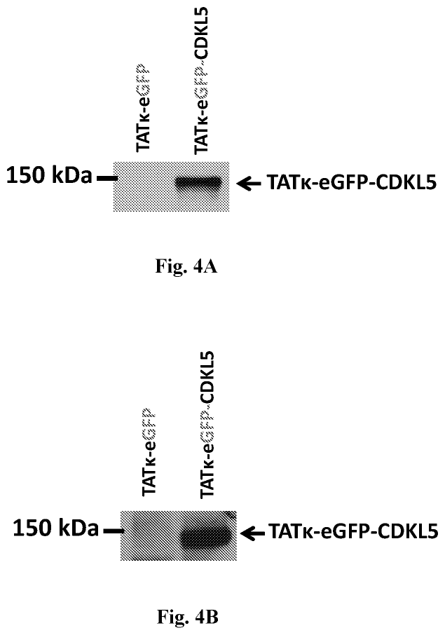

FIGS. 4A and 4B demonstrate western blot analysis results from TATκ-eGFP-CDKL5₁₁₅ protein expression in transfected HEK 293T cells. FIG. 4A demonstrates TATκ-eGFP-CDKL5₁₁₅ fusion protein expression in cell homogenates from transfected HEK 293Tcells. FIG. 4B demonstrates TATκ-eGFP-CDKL5₁₁₅ fusion protein accumulation in concentrated (20×) cell culture medium from transfected HEK 293T cells. Although not shown in FIGS. 4A-4B similar results were obtained with the TATκ-eGFP-CDKL5₁₀₇ fusion protein.

### Example 2: Validation of TATκ-CDKL5₁₁₅ Kinase Activity

In order to purify the TATκ-eGFP-CDKL5₁₁₅ protein, a myc-tag and a 6×His tag were added at the C-terminal region of the TATκ-eGFP-CDKL5₁₁₅ gene. TATκ-eGFP-CDKL5₁₁₅ fusion protein was purified from culture medium on a Ni-NTA resin. It has been shown that the CDKL5 kinase has a high autophosphorylation activity. As shown in FIGS. 5A and 5B, which shows the results from an in vitro kinase activity assay, purified TATκ-eGFP-CDKL5₁₁₅ protein preserves its autophosphorylation activity. This demonstrates that the purified fusion protein retains its kinase activity.

### Example 3: Internalization of TATκ-CDKL5₁₁₅ by HEK 293T Cells

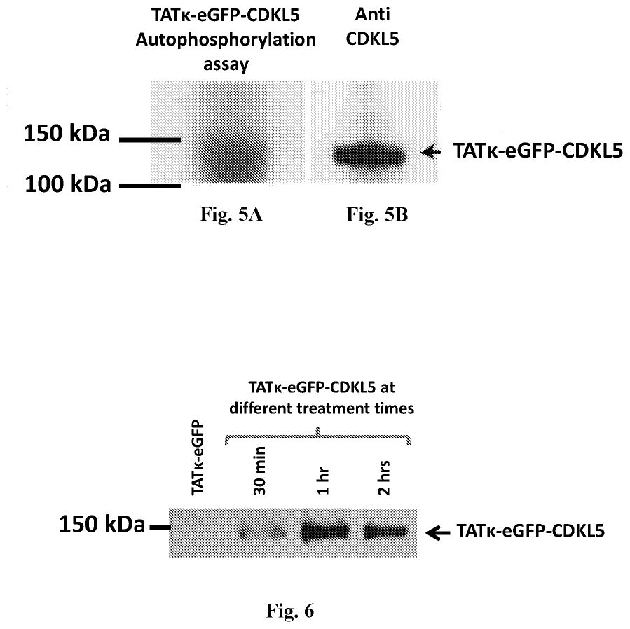

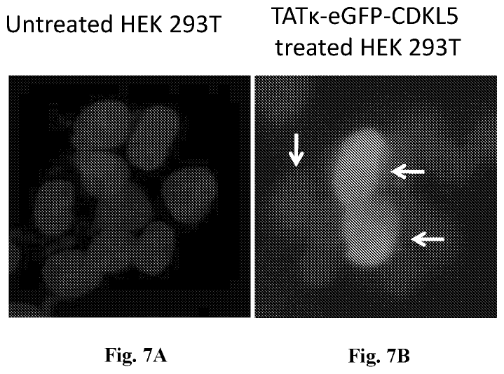

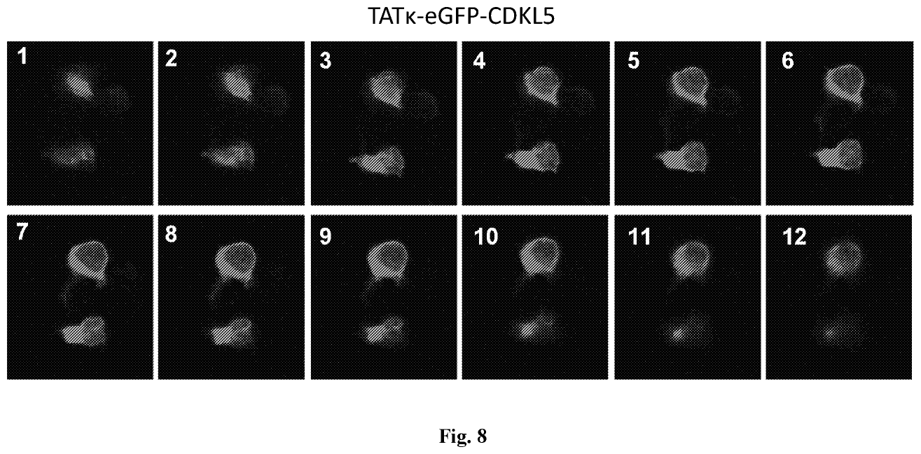

To evaluate the efficiency of the transduction of the TATκ-eGFP-CDKL5₁₁₅ fusion protein HEK 293T cells were incubated with the purified/concentrated fusion protein. Briefly, the TATκ-eGFP-CDKL5₁₁₅ fusion protein was produced and purified as described in Example 1. HEK 293T cells were incubated in concentrated medium containing the fusion protein. After different incubation times cells were lysed and total protein extracts were separated by SDS-PAGE and transferred to a nitrocellulose membrane for immunoblotting for TATκ-eGFP-CDKL5₁₁₅ protein quantification. As shown in FIG. 6, TATκ-eGFP-CDKL5₁₁₅ is internalized by cells after only about 30 minutes of incubation. Other cultures were treated in parallel and were fixed and immunostained with an anti-GFP specific antibody to visualize the transduced TATκ-eGFP-CDKL5₁₁₅ protein. As demonstrated in FIGS. 7A-7B, TATκ-eGFP-CDKL5₁₁₅ protein was efficiently translocated into the cells. The internalization in target cells was confirmed by confocal microscopy (FIG. 8). SH-SY5Y neuroblastoma cells were incubated in concentrated media containing the fusion protein for 30 minutes. FIG. 8 shows an image of a series of confocal images (1-12) of TATκ-eGFP-CDKL5₁₁₅ transduced SH-SY5Y cells, demonstrating that TATκ-eGFP-CDKL5₁₁₅ protein is internalized by target cells and localized both in the nucleus and cytoplasm of SH-SY5Y cells (FIG. 8).

### Example 4: TATκ-CDKL5₁₁₅ Induces Differentiation and Inhibits Proliferation of the SHSY5Y Neuroblastoma Cell Line

In spite of the clear importance of CDKL5 for the central nervous system, the biological functions of this kinase remain largely unknown. The CDKL5 protein can affect both proliferation and differentiation of neural cells (See e.g. Valli et al., 2012. Biochim Biophys Acta. 1819:1173-1185, and Rizzi et al., 2011. Brain Res. 1415:23-33). Neuroblastoma cells share several features with normal neurons and thus are considered a good in vitro model to study the biochemical and functional properties of neuronal cells, particularly when they are induced to differentiate upon treatment with agents such as retinoic acid (RA) (See e.g., Singh, 2007 Brain Res. 1154 p 8-21; Melino, 1997 J. Neurooncol. 31 pp 65-83). For these reasons, neuroblastoma cells were employed to study the CDKL5 function in vitro.

SH-SY5Y cells were treated with purified TATκ-eGFP-CDKL5 similar to the treatment as described in Example 3. Here, SH-SY5Y cells were incubated with the concentrated media containing the purified TATκ-eGFP-CDKL5₁₁₅ protein for about 24 hours. Cell proliferation was evaluated as mitotic index (the ratio between the number of cells in a population undergoing mitosis to the total number of cells) using Hoecsht nuclear staining. Differentiation was evaluated by examining neurite growth, which is a sign of neuronal differentiation. For analysis of neurite growth, cells were grown for an additional 1-2 days in the presence or absence of the pro-differentiation agent, RA. Neurite outgrowth was measured using an image analysis system.

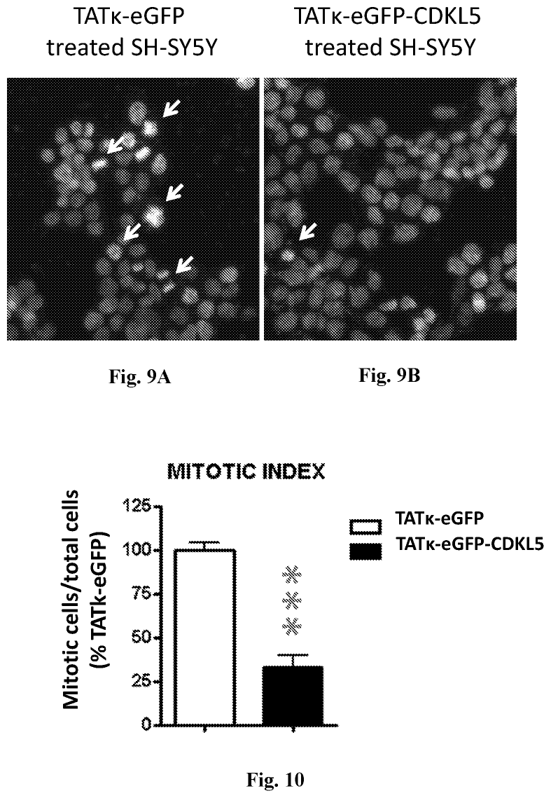

Induction of CDKL5 expression (by TATκ-eGFP-CDKL5₁₁₅ protein) caused a strong inhibition of cell proliferation (e.g., FIGS. 9A-9B, and 10) with no increase in apoptotic cell death (data not shown) compared to controls. Further, as shown in FIGS. 11A-11B and 12, TATκ-eGFP-CDKL5₁₁₅ promotes neuroblastoma cell differentiation as indicated by neurite outgrowth in SH-SY5Y cells. These results demonstrate that TATκ-eGFP-CDKL5₁₁₅ is functional in an in vitro neuronal model.

### Example 5: Characterization of the CDKL5-KO Mouse Model

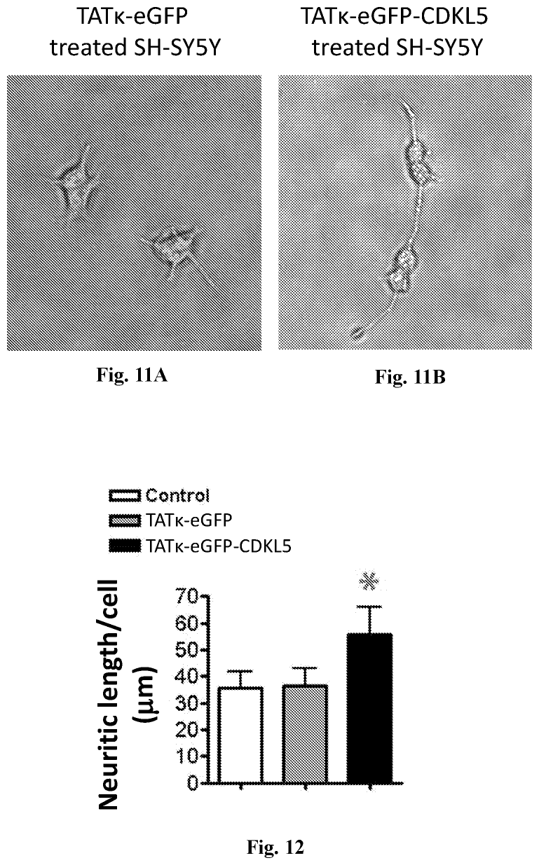

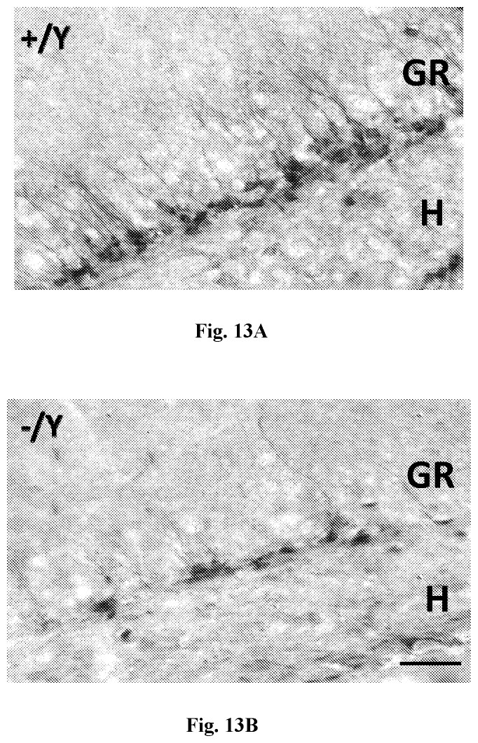

A CDKL5 knockout mouse model has been recently created by the EMBL in Monterotondo, Italy, by the group led by Dr. Cornelius Gross (Amendola, 2014 PLoS One. 9(5):e91613). To establish the effect of CDKL5-loss of function on dendritic development of newborn neurons, the dendritic morphology of newborn hippocampal granule cells derived from the CDKL5 KO mouse was examined. Dendritic morphology of newborn neurons was analyzed with immunohistochemistry for doublecortin (DCX), taking advantage of the expression of this protein in the cytoplasm of immature neurons during the period of neurite elongation. As shown in FIGS. 13A-13B, DCX-positive cells of CDKL5 KO mice (−/Y) exhibited a dendritic tree with a highly immature pattern (FIG. 13B) compared to the CDKL5 wild-type (+/Y) counterparts (FIG. 13A). A highly immature pattern can be evidenced by little branching and elongation. Absence of CDKL5 resulted in a decrease in the number of DCX-positive cells (FIG. 13B) due to an increase in apoptotic cell death (data not shown) that was observed to affect postmitotic immature granule neurons (DCX-positive cells) (Fuchs, 2014 Neurobiol Dis. 70 p53-68). These data suggests that CDKL5 plays a fundamental role on postnatal neurogenesis, by affecting neural precursor survival and maturation of newborn neurons. Cultures of neuronal precursor cells (NPCs) from the subventricular zone (SVZ) of CDKL5 knockout mice were observed to exhibit the same defects observed in vivo in cerebellar granule cell precursors. Namely, in cultures of neuronal precursor cells derived from female wild-type mice (+/+) there were more neurons (β-tubulin III positive cells, red cells) than in cultures of neuronal precursor cells derived from homozygous CDKL5 KO female mice (−/−) (FIGS. 14A and 14B). This suggests that the loss of CDKL5 decreases the survival of post-mitotic neurons. Assessment of neurite outgrowth in β-tubulin III positive cells demonstrated that neurons generated from Cdk15 knockout NPCs were less differentiated compared to female wild-type (+/+) neurons (FIGS. 14A and 14B). These results suggest that post-mitotic NPCs from CDKL5 knockout mice have an intrinsic defect, not only in cell survival, but also in neuronal maturation.

### Example 6: TATκ-CDKL5₁₁₅ Protein Restores Neurite Development of Neuronal Cell Precursors Derived from a CDKL5 KO Mouse

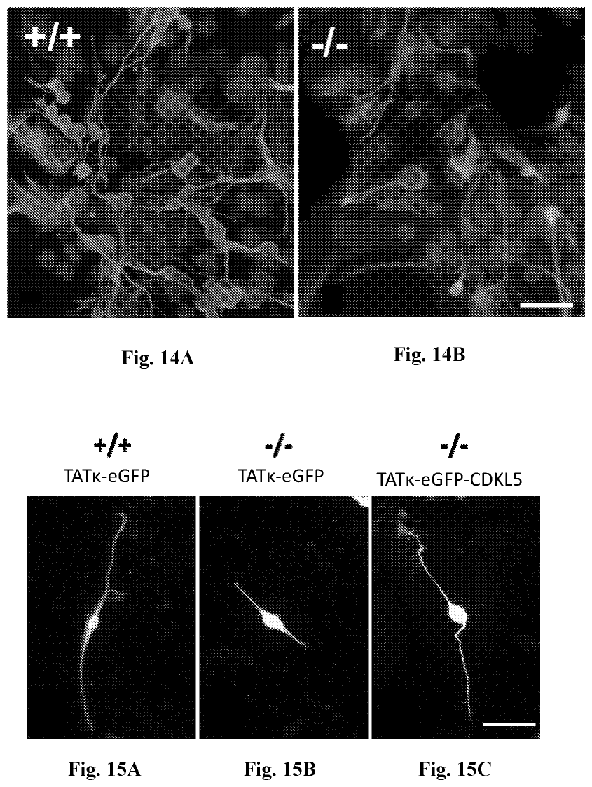

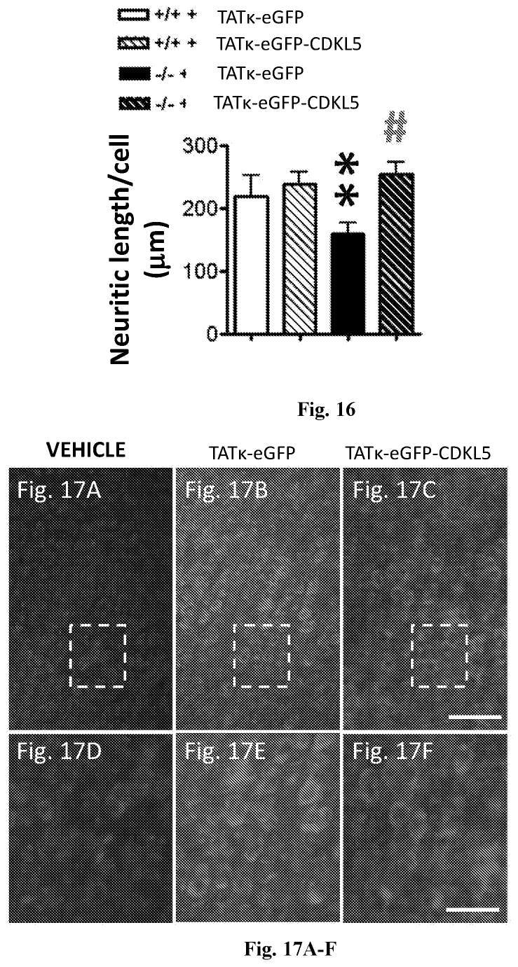

Neuronal precursor cells cultures from the female homozygous CDKL5 KO (−/−) mouse and wild-type (+/+) mouse were treated with TATκ-eGFP-CDKL5₁₁₅ or TATκ-eGFP. Neuronal maturation was evaluated by measuring the total neuritic length of differentiated neurons (positive for β-tubulin III). Evaluation of neurite length was performed by using the image analysis system Image Pro Plus (Media Cybernetics, Silver Spring, Md. 20910, USA). The average neurite length per cell was calculated by dividing the total neurite length by the number of cells counted in the areas. As shown in FIGS. 15A-15C and 16, absence of CDKL5 causes a reduction in the maturation of new neurons and treatment with TATκ-eGFP-CDKL5₁₁₅ restores neurite development. For FIGS. 15A-16, cells were isolated from the subventricular zone (SVZ) of newborn (2-day-old) mice. For differentiation analysis, neurospheres obtained after three passages in vitro were dissociated and plated on cover slips coated with 15 μg/ml poly-1-ornithine (Sigma) at a density 20,000 cells/well. Cells were grown for 2 days and then transferred to a differentiating medium (EGF and FGF free plus 1% foetal bovine serum) from day 3 for 7 days. TATκ-CDKL5₁₁₅ fusion protein was administered daily at a final 10× concentration after buffer exchange with DMEM-F12, avoiding complete change of culture medium. Every 3 days, half of the medium was replenished with fresh differentiating medium.

### Example 7: Delivery of TATκ-CDKL5₁₁₅ into the Mouse Brain

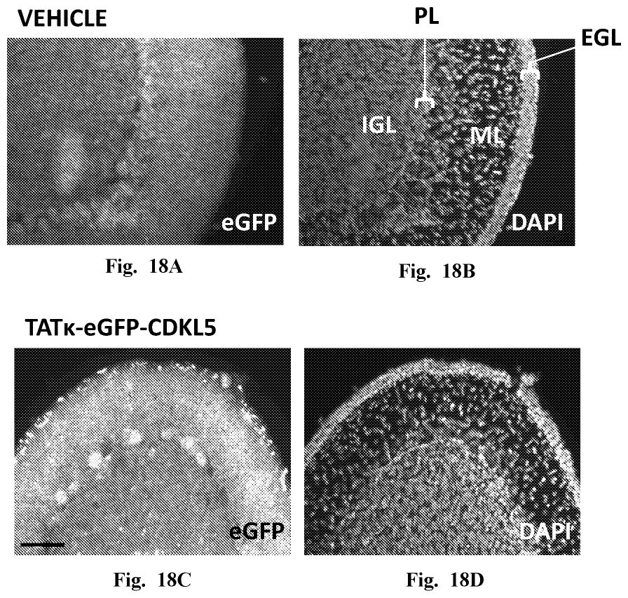

Seven-day old mouse pups were subcutaneously injected with a single dose of culture medium of HEK 293T cells transfected with TATκ-eGFP-CDKL5₁₁₅, TATκ-eGFP or medium from untransfected cells (vehicle) (single dose corresponded to about 200 μl of 200× concentrated medium; which contained about 1-1.5 μg of the fusion protein). Culture medium was collected after 48 hours from transfection and was diafiltered and concentrated with Amicon ultra centrifugal filters (50 kDa cut-off). Mice were sacrificed 4 hours post-administration of the treatment. Brains were stored in the fixative for 24 hours, cut along the midline and kept in 20% sucrose in phosphate buffer for an additional 24 hours. Hemispheres were frozen and stored at −80° C. The right hemisphere was cut with a freezing microtome in 30-μm-thick coronal sections. Immunohistochemistry was carried out on free-floating sections. Localization of TATκ-eGFP-CDKL5₁₁₅ and TATκ-eGFP in the brain was evaluated by immunohistochemistry using an anti-GFP antibody and a TSA amplification kit. Images were taken at the level of the sensory-motor cortex and the cerebellum. Cells were counterstained using 4′,6-diamidino-2-phenylindole (DAPI). Representative images demonstrating presence of the TATκ-eGFP-CDKL5₁₁₅ protein in the sensory-motor cortex and cerebellum of mice are shown in FIGS. 17A-17F and FIGS. 18A-18D, respectively. Given that the TATκ-eGFP-CDKL5₁₁₅ protein was subcutaneously administered, these data demonstrates that the TATκ-eGFP-CDKL5₁₁₅ protein is effectively transported across the blood brain barrier and enters into brain cells.

### Example 8: Effect of TATκ-CDKL5₁₁₅ Fusion Protein In Vivo on Neuronal Maturation, Survival and Connectivity

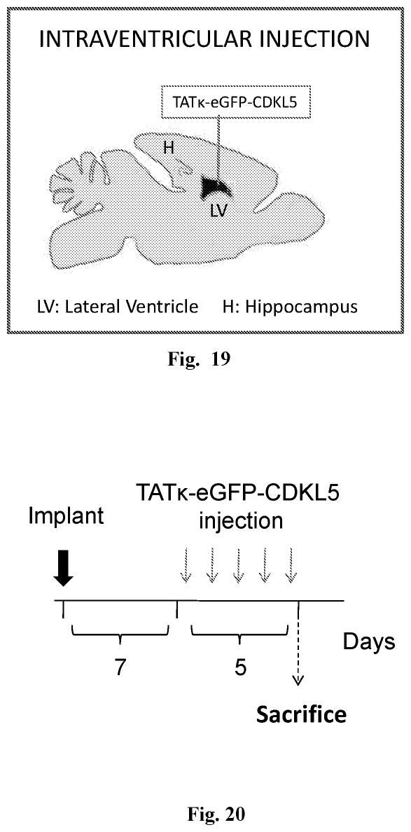

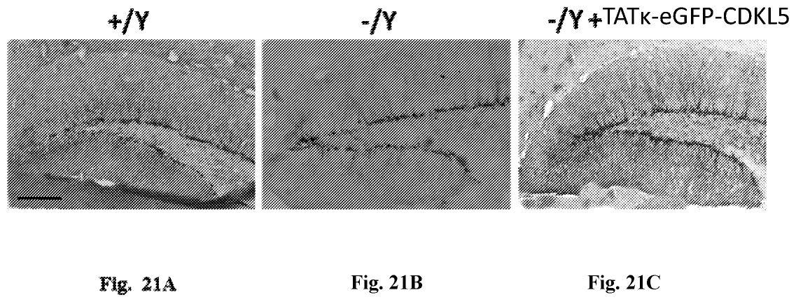

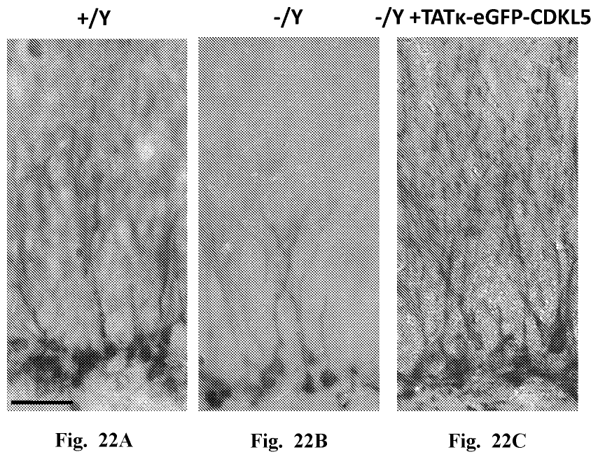

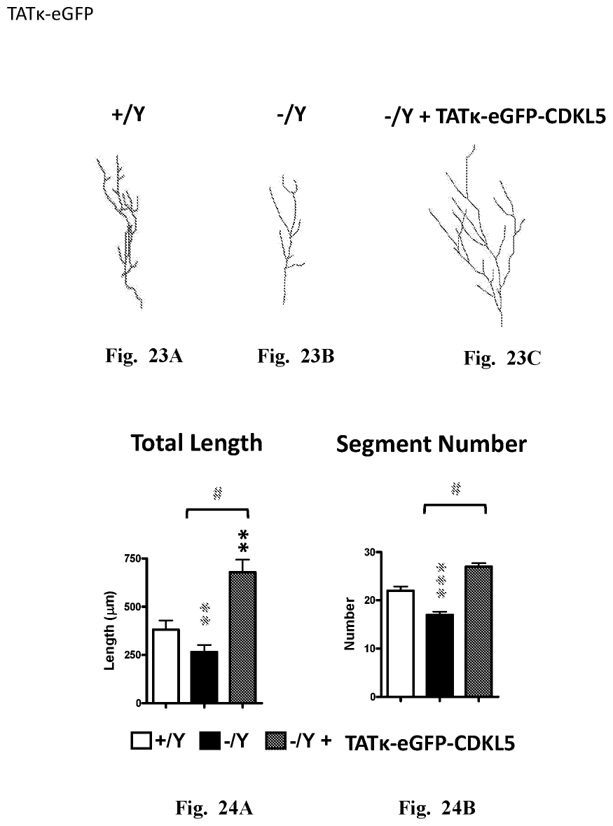

Adult mice (4-6 months of age) were intraventricularly injected (FIG. 19) for 5 consecutive days (see e.g. FIG. 20 for experimental schedule) with TATκ-eGFP-CDKL5₁₁₅ or TATκ-eGFP. Briefly, mice were anesthetized with ketamine (100-125 mg/kg) and xylazine (10-12.5 mg/kg). Cannulas (0.31-mm diameter, Brain Infusion Kit III; Alzet Cupertino, Calif.) were stereotaxically implanted into the lateral ventricles (A/P −0.4-mm caudal, M/L 1.0 mm, D/V −2.0 mm; FIG. 19). Seven days after implantation mice were infused for 5 consecutive days with 10 μl (about 50 ng) of TATκ-eGFP-CDKL5₁₁₅ or TATκ-eGFP in PBS by using a Hamilton syringe connected to a motorized nanoinjector (at a rate of 0.5 μl/min). Four hours after the last injection animals were sacrificed, and the dendritic morphology of newborn hippocampal granule cells was analyzed with immunohistochemistry for DCX. FIGS. 21A-21C and 22A-22C demonstrate that DCX positive neurons of male CDKL5 KO mice had shorter processes than those of their wild-type counterparts (FIGS. 21A-21B and 22A-22B). TATκ-eGFP-CDKL5₁₁₅ fusion protein administered intraventricularly on five consecutive days was observed to increase neurite length and branch number in CDKL5 knockout male mice (FIG. 22C) to levels similar to wild-type (FIG. 22A). FIGS. 23A-23B show examples of the reconstructed dendritic tree of newborn granule cells of wild-type (+/Y) (FIG. 23A), CDKL5 knockout male mice (−/Y) (FIG. 23B), and CDKL5 knockout male mice treated with a TATκ-eGFP-CDKL5₁₁₅ fusion protein.

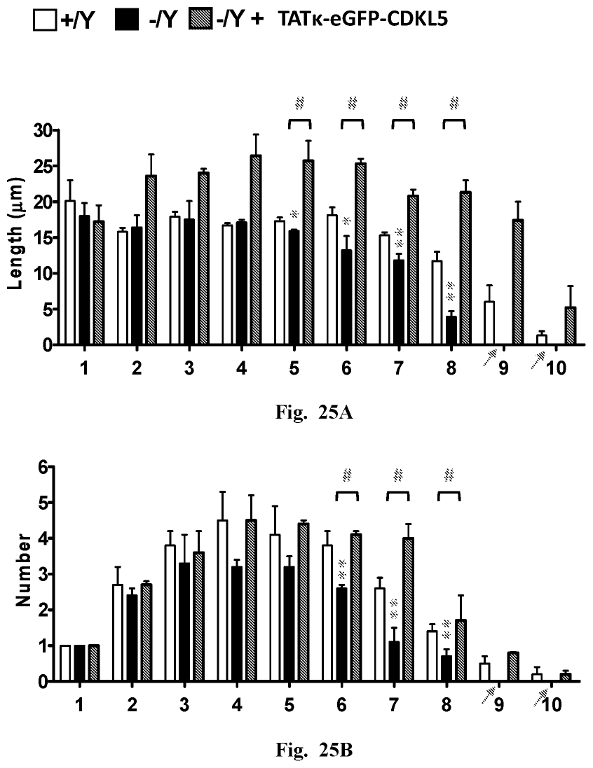

Quantification of the dendritic size of DCX positive cells demonstrate that CDKL5 KO male mice (−/Y) mice had a shorter dendritic length (FIG. 24A) and a reduced number of segments (FIG. 24B) than wild-type male mice (FIGS. 24A and 24B). In TATκ-eGFP-CDKL5₁₁₅ treated CDKL5 knockout male mice there was an increase in both parameters that became even larger in comparison with wild-type male mice (FIGS. 24A-24B). The effects of TATκ-eGFP-CDKL5₁₁₅ treatment on details of the dendritic architecture were examined by evaluating each dendritic order separately. A striking feature of CDKL5 KO mice was the absence of branches of higher order (FIGS. 25A-25B; red arrows). While wild-type male mice had up to 10 orders of branches, CDKL5 knockout male mice lacked branches of orders 8-10 (FIG. 25A, arrows). In addition, CDKL5 knockout male mice showed a reduced branch length of orders 5-8 (FIG. 25A) and a reduced number of branches of orders 6-8 (FIG. 25B). Taken together, these data indicate that in CDKL5 KO male mice the dendritic tree of the newborn granule cells is hypotrophic and that this defect is due to a reduction in the number and length of branches of intermediate order and a lack of branches of higher order. All these defects were observed to be completely rescued by TATκ-eGFP-CDKL5₁₁₅ treatment (FIGS. 25A to 25B).

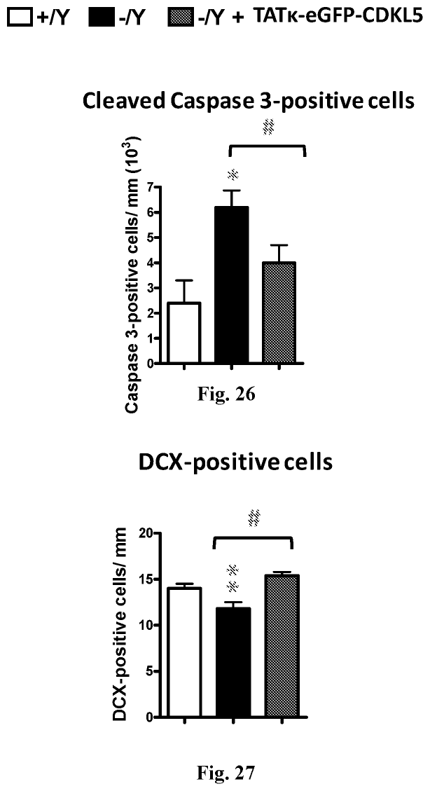

In order to evaluate the effect of TATκ-eGFP-CDKL5₁₁₅ treatment on apoptotic cell death, we counted the number of apoptotic cells expressing cleaved caspase-3 in the hippocampal dentate gyrus (FIG. 26). Quantification of cleaved caspase-3 cells shows that TATκ-eGFP-CDKL5₁₁₅ treatment completely normalized apoptotic cell death in CDKL5 knockout male mice (−/Y) (FIG. 26). It was observed that CDKL5 knockout male mice had fewer post-mitotic neurons (DCX-positive cells) than wild-type male mice in the hippocampal dentate gyrus (FIG. 27). TATκ-eGFP-CDKL5₁₁₅-treated CDKL5 knockout mice underwent an increase in the number of post-mitotic neurons that became similar to those of wild-type male mice (FIG. 27). This indicates that the increased death of post-mitotic immature granule cells that characterizes CDKL5 knockout mice is rescued by TATκ-eGFP-CDKL5₁₁₅ treatment. Taken together, these data demonstrate that treatment with TATκ-eGFP-CDKL5₁₁₅ in CDKL5 knockout mice increased neurite length and survival of newborn cells in the hippocampus indicating that injected TATκ-CDKL5 diffused from the lateral ventricle to the hippocampus and restored maturation and survival of post-mitotic granule cells.

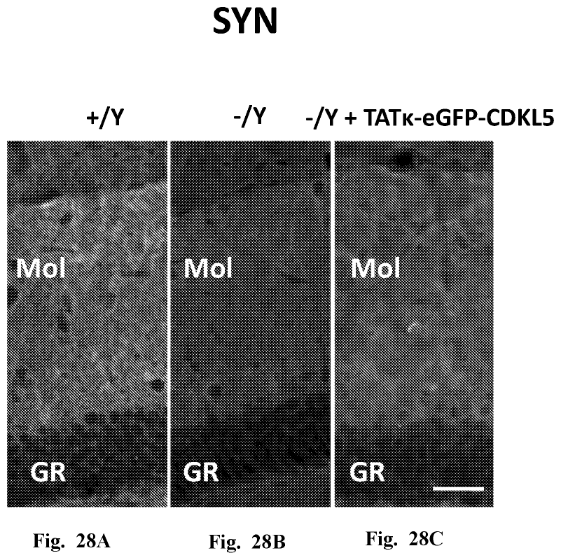

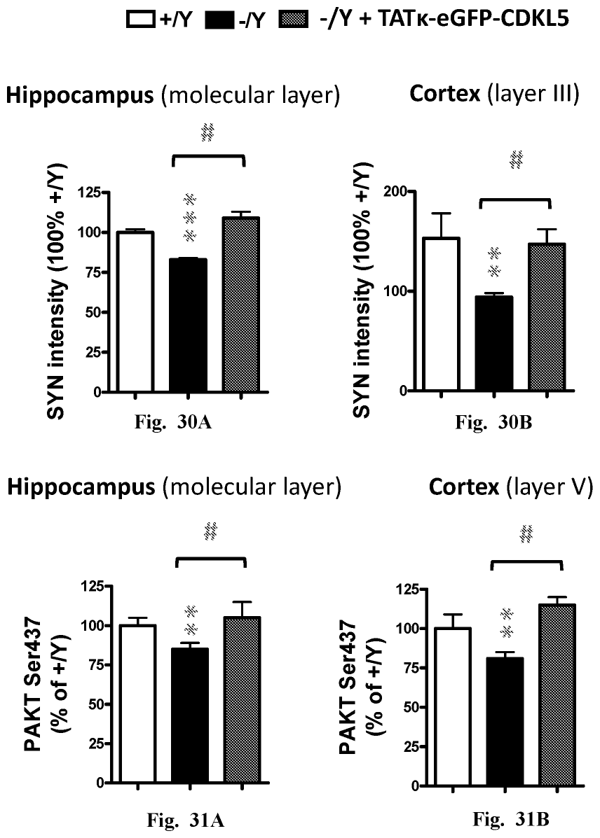

Without being bound by any one theory, a reduction in connectivity may be the counterpart of the dendritic hypotrophy that characterizes the newborn granule cells of CDKL5 KO mice. Synaptophysin (SYN; also known as p38) is a synaptic vesicle glycoprotein that is a specific marker of presynaptic terminals. Here, it was observed in CDKL5 knockout male mice that the optical density of SYN was significantly lower than in wild-type male mice in the molecular layer of the hippocampus (FIGS. 28 and 30A), suggesting that CDKL5 KO male mice had fewer synaptic contacts in the dentate gyrus. FIGS. 28A-28C show representative images demonstrating brain sections processed for synaptophysin (SYN) immunofluorescence from the molecular layer (Mol) of the dentate gryrus (DG) from a wild-type male mouse (+/Y) (FIG. 28A), a CDKL5 knockout male mouse (−/Y) (FIG. 28B), and a CDKL5 knockout male mouse treated with TATκ-eGFP-CDKL5₁₁₅ fusion protein via intraventricular injections given once a day for 5 consecutive days (−/Y+TATκ-eGFP-CDKL5) (FIG. 28C). One out of six 30-μm-thick coronal sections from the DG of animals were processed for immunohistochemistry. Immunohistochemistry was carried out on free-floating sections for the frozen brains. For Synaptophysin immunohistochemistry, sections were incubated for 48 hours at 4° C. with mouse monoclonal anti-SYN (SY38) antibody (1:1000, MAB 5258, Millipore Bioscience Research Reagents) and for 2 hours with a Cy3 conjugated anti-mouse IgG secondary antibody (1:200; Jackson Immunoresearch). Intensity of immunoreactivity (IR) was determined by optical densitometry of immunohistochemically stained sections. Fluorescence images were captured using a Nikon Eclipse E600 microscope equipped with a Nikon Digital Camera DXM1200 (ATI system). Densitometric analysis in the molecular layer and cortex was carried out using Nis-Elements Software 3.21.03 (Nikon). For each image, the intensity threshold was estimated by analyzing the distribution of pixel intensities in the image areas that did not contain IR. This value was then subtracted to calculate IR of each sampled area. Values are given as a percentage of the optical density of control CDKL5 wild-type male mice (mean+standard error).

Dendritic arborization is significantly reduced in cortical pyramidal neurons of CDKL5 KO mice compared to their wild-type counterparts (Amendola, 2014 PLoS One. 9(5):e91613). A similar lower level of SYN immunoreactivity in the layer III of the neocortex was observed (FIG. 30B). In CDKL5 KO male mice treated with TATκ-eGFP-CDKL5₁₁₅ these defects were fully rescued (FIG. 28 and FIGS. 30A and 30B), suggesting that the positive impact of treatment with TATκ-eGFP-CDKL5₁₁₅ on dendritic structure was paralleled by restoration of the input to neurons.

### Example 9: Effect of TATκ-CDKL5₁₁₅ Fusion Protein In Vivo on P-AKT

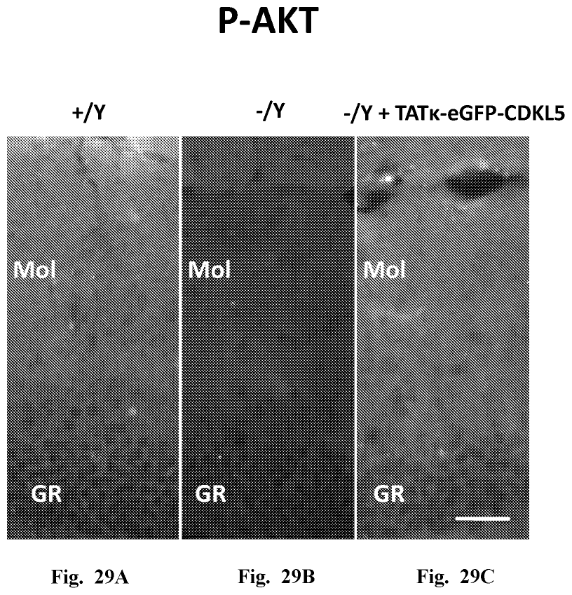

AKT is a central signaling kinase associated with multiple cellular pathways. Phosphorylated AKT (P-AKT) is significantly reduced in CDKL5 knockout animals, CDKL5 deficiency and Rett syndrome. FIGS. 29A-29C show representative images demonstrating brain sections processed for P-AKT immunofluorescence from the molecular layer (Mol) of the dentate gryrus (DG) from a wild-type male mouse (+/Y) (FIG. 29A), a CDKL5 knockout male mouse (−/Y) (FIG. 29B), and a CDKL5 knockout male mouse treated with TATκ-eGFP-CDKL5₁₁₅ fusion protein via intraventricular injections given once a day for 5 consecutive days (−/Y+TATκ-eGFP-CDKL5) (FIG. 29C). For phospho-AKT immunohistochemistry, sections were incubated for 24 hours at 4° C. with mouse monoclonal anti-phospho-AKT-Ser473 antibody (1:1000, Cell Signaling Technology) and for 2 hours with a Cy3 conjugated anti-mouse IgG secondary antibody (1:200; Jackson Immunoresearch). Intensity of immunoreactivity (IR) was determined by optical densitometry of immunohistochemically stained sections. Fluorescence images were captured using a Nikon Eclipse E600 microscope equipped with a Nikon Digital Camera DXM1200 (ATI system).

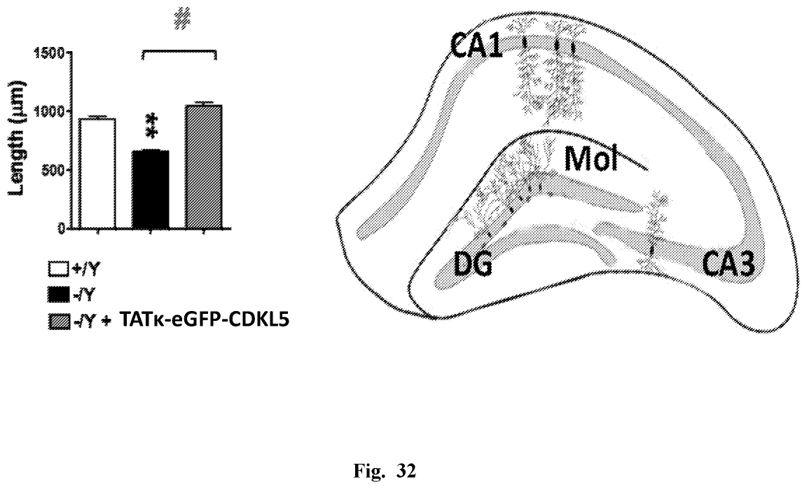

In CDKL5 knockout male mice (−/Y) the optical density of P-AKT in the molecular layer of the DG (FIG. 31A) and in the layer V of the cortex (FIG. 31B) was observed to be significantly lower than in +/Y mice. In CDKL5 knockout male mice (−/Y) intraventricular injected with TATκ-eGFP-CDKL5₁₁₅ for five consecutive days these defects were fully rescued (FIGS. 31A and 31B), demonstrating that treatment with TATκ-eGFP-CDKL5₁₁₅ in CDKL5 knockout mice restores AKT activity.

### Example 10: Effect of TATκ-CDKL5₁₁₅ Fusion Protein In Vivo on Dendritic Architecture of Mature Neurons

The effects of treatment on the dendritic architecture of mature neurons were analyzed. To this end Golgi-stained granule neurons located in the middle portion of the granule cell layer were examined. While CDKL5 KO male mice show a shorter length of dendritic branches compared to wild-type male mice, these defects were completely rescued by treatment with TATκ-eGFP-CDKL5₁₁₅ (FIG. 32). In both treated (−/Y) and (+/Y) mice total dendritic length became even larger in comparison with untreated (+/Y) mice. These results show that the impaired dendritic architecture of mature granule neurons observed in CDKL5 KO male mice was restored by treatment with TATκ-eGFP-CDKL5₁₁₅ protein.

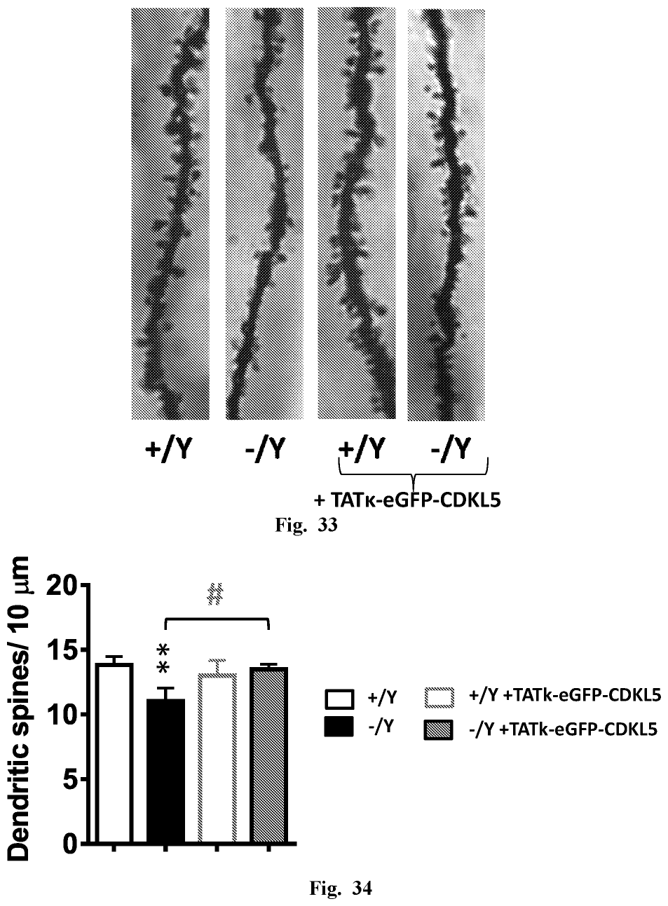

In Golgi-stained sections, spines of granule cells were counted and spine density was measured on dendritic segments in the inner and outer half of the molecular layer. Untreated CDKL5 KO adult male mice showed a lower spine density compared to wild-type mice, while treatment with TATκ-eGFP-CDKL5₁₁₅ protein fully restored density, erasing the difference between knockout and wild-type conditions. Representative images are shown in FIG. 33 and the relative quantification can be observed in the histogram on the right FIG. 34.

Taken together, these data demonstrate that treatment with TATκ-eGFP-CDKL5₁₁₅ completely recovers connectivity in CDKL5 KO male mice, by restoring synaptophysin expression and by correcting dendritic spine number and maturation.

### Example 11: Effect of TATκ-CDKL5₁₁₅ Fusion Protein on Learning and Memory Ability

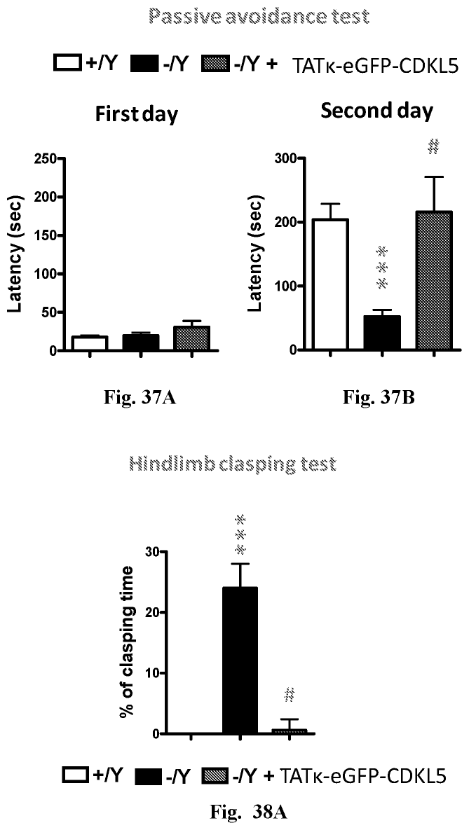

Male CDKL5 knockout mice exhibit learning and memory deficits as compared to wild-type mice (see e.g. FIGS. 36, and 37A-37B).

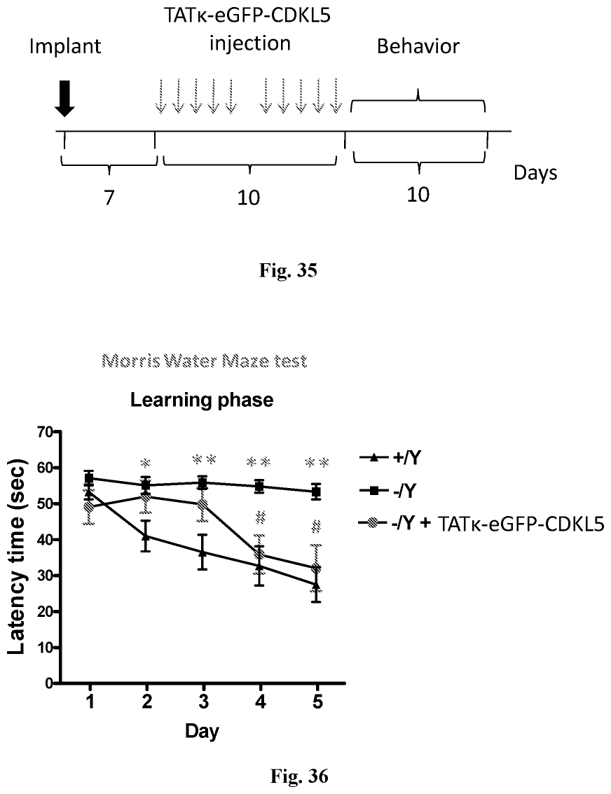

To examine memory and learning ability, CDKL5 knockout male mice were administered daily intraventricular injections of a TATκ-eGFP-CDKL5₁₁₅ fusion protein for 10 days (see e.g. FIG. 35 for experimental schedule). After a two day rest period at the conclusion of 10 days of injections, mice in all groups received the Morris Water Maze (MWM) testing (FIG. 36). MWM measures the ability to find and recall the position of a hidden platform submerged in water. Mice were trained in the MWM task to locate a hidden escape platform in a circular pool. The apparatus consisted of a large circular water tank (1.00 m diameter, 50 cm height) with a transparent round escape platform (10 cm²). The pool was virtually divided into four equal quadrants identified as northeast, northwest, southeast, and southwest. The tank was filled with tap water at a temperature of 22° C. up to 0.5 cm above the top of the platform and the water was made opaque with milk. The platform was placed in the tank in a fixed position (in the middle of the north-west quadrant). The pool was placed in a large room with a number of intra- (squares, triangles, circles and stars) and extra-maze visual cues. After training, each mouse was tested for two sessions of 4 trials each per day, for 5 consecutive days with an inter-session interval of 40 minutes (acquisition phase). A video camera was placed above the center of the pool and connected to a videotracking system (Ethovision 3.1; Noldus Information Technology B.V., Wageningen, Netherlands). Mice were released facing the wall of the pool from one of the following starting points: North, East, South, or West and allowed to search for up to 60 seconds for the platform. If a mouse did not find the platform, it was gently guided to it and allowed to remain there for 15 seconds. The latency to find the hidden platform was used as a measure of learning. All experimental sessions were carried out between 9:00 a.m. and 3:00 p.m.

The results of this test are demonstrated in FIG. 36. FIG. 36 shows a graph demonstrating the quantification of the learning phase as determined via the Morris Water Maze test in wild-type male mice (+/Y), CDKL5 KO male mice (−/Y), and CDKL5 KO male mice treated with a TATκ-eGFP-CDKL5₁₁₅ fusion protein (−/Y+TATκ-eGFP-CDKL5). Wild-type mice learned to find the platform by the second day, but no significant learning was detected in CDKL5 KO mice. CDKL5 KO male mice treated with a TATκ-eGFP-CDKL5₁₁₅ fusion protein began to recover learning ability at day 4 with continued improvement at day 5.

Memory and learning ability as further examined in response to TATκ-eGFP-CDKL5₁₁₅ fusion protein treatment using a passive avoidance test. After 10 consecutive days of treatment and a two day rest period, mice of the various groups received passive avoidance testing (FIG. 37). The experiment utilized a test cage with two chambers (light and dark). On day one (conditioning period), animals are placed in the light chamber and instinctively move into the dark chamber where they are conditioned with a single adverse event (foot shock). For the passive avoidance test we used a tilting-floor box (47×18×26 cm) divided into two compartments by a sliding door and a control unit incorporating a shocker (Ugo Basile, Italy). This classic instrument for Pavlovian conditioning exploits the tendency in mice to escape from an illuminated area into a dark one (step-through method). On the first day mice were individually placed into the illuminated compartment. After a 60 second habituation period, the connecting door between the chambers opened. In general, mice step quickly through the gate and enter the dark compartment because mice prefer to be in the dark. Upon entering the dark compartment, mice received a brief foot shock (0.7 mA for 3 seconds) and were removed from the chamber after 15 seconds of latency. If the mouse remained in the light compartment for the duration of the trial (358 s), the door closed and the mouse was removed from the light compartment. The chambers were cleaned with 70% ethanol between testing of individual mice. After a 24 hour retention period, mice were placed back into the light compartment and the time it took them to re-enter the dark compartment (latency) was measured up to 358 seconds.

FIGS. 37A-37B demonstrate the results from the passive avoidance test. FIG. 37A indicates that the latency time to enter the dark chamber was similar for all groups. On day two (testing period) (FIG. 37B) animals were again placed in the light chamber. Memory of the adverse event was measured by latency time to enter the dark chamber. CDKL5 knockout male mice (−/Y) were severely impaired at performing this task, as demonstrated by a reduced latency to enter the dark compartment in comparison with GDKL5 male wild-type mice (+/Y). TATκ-eGFP-CDKL5₁₁₅ treated CDKL5 knockout male mice showed a similar latency time as compared to wild-type mice.

In sum, the data demonstrate that TATκ-eGFP-CDKL5₁₁₅ can increase and restore learning and memory ability in CDKL5 knockout male mice to levels similar to that observed in their untreated wild-type counterparts.

### Example 12: Effect of TATκ-CDKL5₁₁₅ Fusion Protein on Motor Function

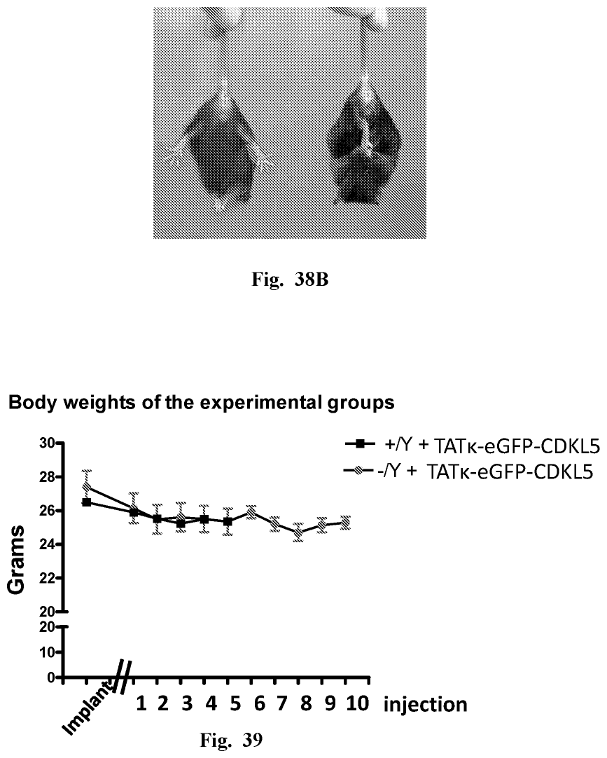

CDKL5 knockout male mice exhibited prolonged limb clasping when suspended (see e.g. FIGS. 38A-38B).

To examine the effect of TATκ-eGFP-CDKL5₁₁₅ fusion protein on motor function, mice were administered daily intraventricular injections of TATκ-eGFP-CDKL5₁₁₅ for 10 consecutive days (FIG. 38). 10 days following the completion of the dosing protocol, animals were suspended in the air by the tail (FIG. 38A and FIG. 38B). All animals were suspended for about 2 minutes and total time of limb clasping was measured. Results from this experiment are demonstrated in FIGS. 38A-38B.

FIGS. 38A-38B show a graph demonstrating quantification of motor ability as determined by a clasping test in which total amount of time spent limb clasping during a 2 minute interval was measured in wild-type male mice (+/Y), CDKL5 knockout male mice (−/Y), and CDKL5 KO male mice treated with a TATκ-eGFP-CDKL5₁₁₅ fusion protein (−/Y+TATκ-eGFP-CDKL5) according to the injection schedule in FIG. 35. In sum, the data demonstrate that treatment with TATκ-eGFP-CDKL5₁₁₅ improved motor function in CDKL5 KO male mice.

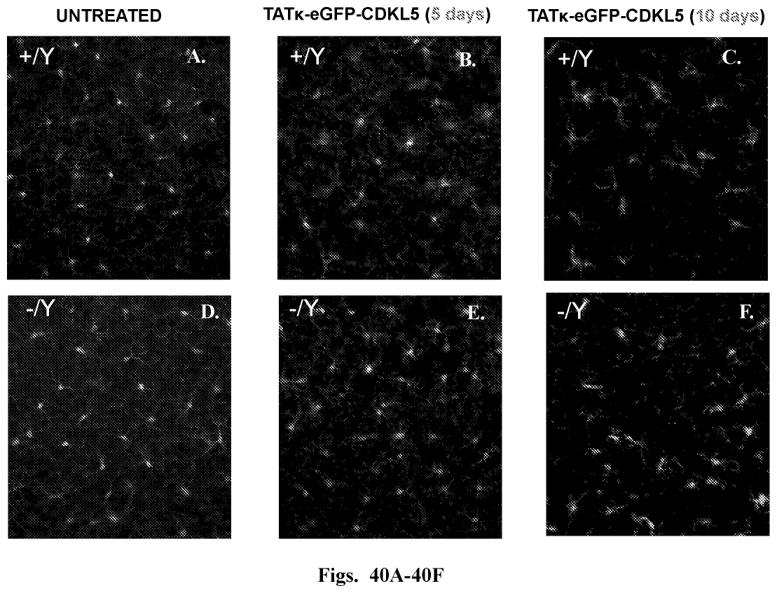

Body weight of wild-type (+/Y) and CDKL5 KO (−/Y) male mice injected for 5 (+/Y) or 10 (−/Y) days with TATκ-eGFP-CDKL5₁₁₅ protein was measured and results are demonstrated in FIG. 39. No significant changes in body weight during the injection period were observed, suggesting that there were no side effects caused by TATκ-eGFP-CDKL5₁₁₅ protein administration.

Comparison of Allograft Inflammatory Factor 1 (AIF-1) staining in untreated animals and treated with TATκ-eGFP-CDKL5₁₁₅ for 5 days or 10 days by intraventricular injection is shown in FIGS. 40A-40F. Data show that treatment does not provoke microglial activation, suggesting the absence of inflammatory response to the prolonged TATκ-eGFP-CDKL5₁₁₅ treatment.

### Example 13: Comparison of Expression and Activity Between TATκ-CDKL5 Isoforms

Alternative splice isoforms for the CDKL5 gene have been described (Kilstrup-Nielsen, 2012). The original CDKL5 transcript generates a protein of 1030 amino acids (CDKL5₁₁₁₅; 115 kDa). While CDKL5₁₁₅ was the first characterized CDKL5 isoform, a recently identified 107 kDa isoform has been demonstrated to contain an altered C-terminal region and is thought to be involved with brain function (CDKL5₁₀₇) (Williamson et al., 2012). As described above, the CDKL5 fusion protein can be formed using any suitable isoform (e.g. a variant as described elsewhere herein). For this Example, the CDKL5 fusion protein was formed by operatively liking the CDKL5₁₁₅ isoform (SEQ ID NO: 2) or the CDKL5₁₀₇ isoform (SEQ ID NO: 16) to TATκ using similar methods described elsewhere herein.

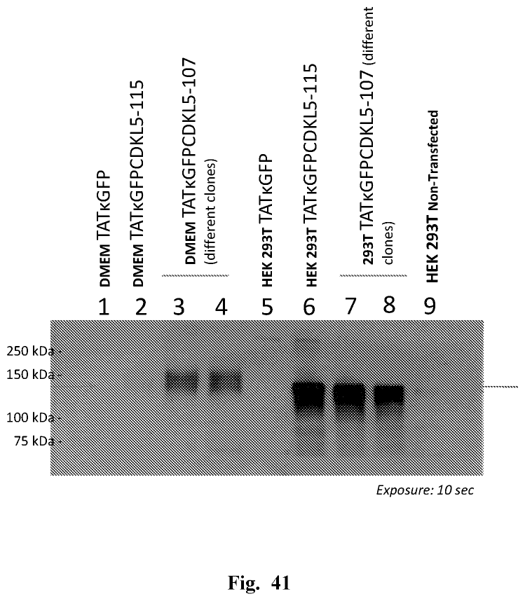

To compare the levels of production and activity of the two CDKL5 isoforms (115 and 107), TATκ-eGFP-CDKL5₁₁₅ or TATκ-eGFP-CDKL5₁₀₇ was transiently or stably expressed in HEK 293T cells. It was observed that the recovery of TATκ-eGFP-CDKL5₁₀₇ fusion protein from culture medium was higher than that of TATκ-eGFP-CDKL5₁₁₅ (FIG. 41).

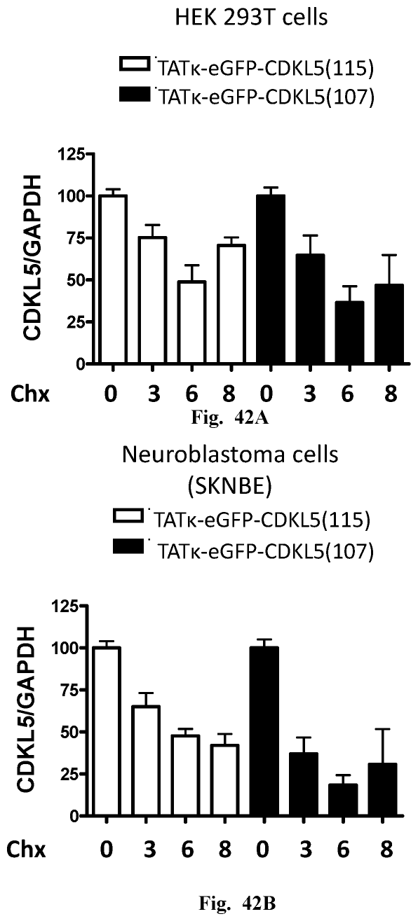

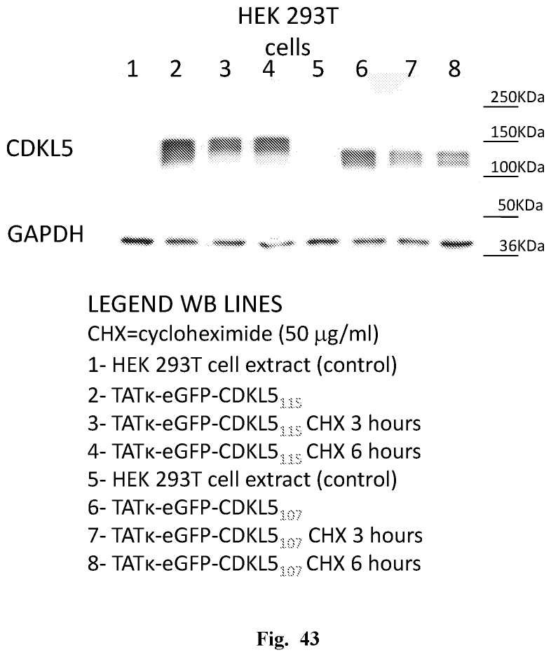

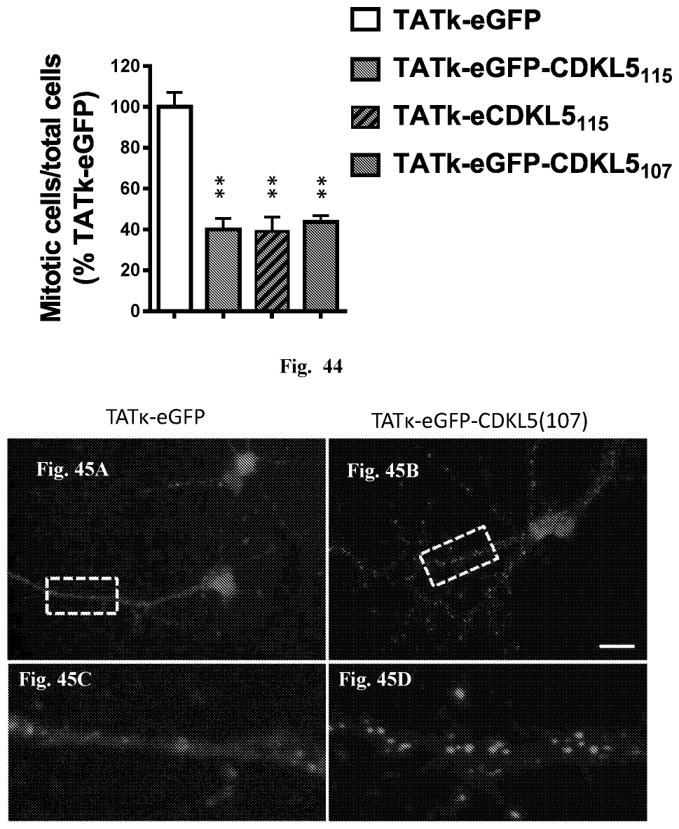

It was observed that the two CDKL5 isoforms have similar intracellular stability. FIGS. 42A-42B show graphs demonstrating intracellular stability of expressed TATκ-eGFP-CDKL5₁₁₅ or TATκ-eGFP-CDKL5₁₀₇ fusion proteins in HEK 293T cells (FIG. 42A) and SKNBE cells (FIG. 42B). HEK 293T and SKNBE cells were transfected with TATκ-eGFP-CDKL5₁₁₅ or TATκ-eGFP-CDKL5₁₀₇. Twenty-four hours later cells were incubated with cycloheximide (Chx; 50 μg/ml) for the indicated times (3, 6 or 8 hours). Ectopically expressed CDKL5 was detected by CDKL5 immunoblotting.

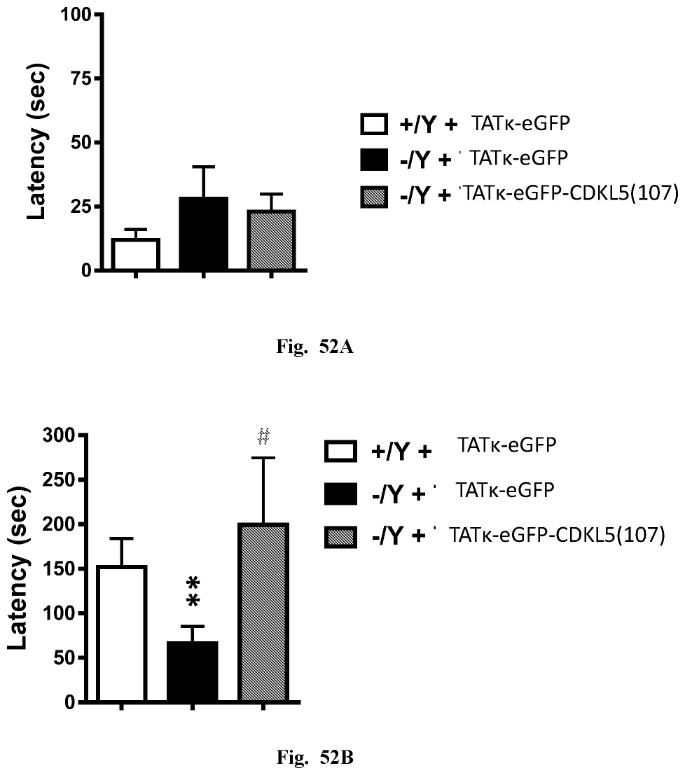

Importantly, at a functional level, we found that the two isoforms have a comparable physiological activity. The in vitro activity of TATκ-eGFP-CDKL5₁₁₅ was tested in parallel to a TATκ-CDKL5₁₁₅ and TATκ-eGFP-CDKL5₁₁₅. SH-SY5Y cells were treated with purified TATκ-CDKL5₁₁₅ or TATκ-eGFP-CDKL5₁₁₅ and TATκ-eGFP (as a control) the day after seeding. In particular, cells were incubated with the concentrated media containing the concentrated/purified protein for about 24 hours. Cell proliferation was evaluated as a mitotic index (the ratio between the number of cells in a population undergoing mitosis to the total number of cells) using Hoechst nuclear staining. As shown in FIG. 52, we demonstrated that both CDKL5 isoforms have the same effect on SH-SY5Y neuroblastoma cells in terms of inhibition of proliferation. We tested the in vitro activity of TATκ-eGFP-CDKL5₁₁₅ in parallel to a TATκ-CDKL5₁₁₅ protein without eGFP. As shown in FIG. 52, we demonstrated that both the proteins have the same effect on SH-SY5Y neuroblastoma cells in terms of inhibition of proliferation, indicating that the eGFP-tag does not alter CDKL5 activity.

### Example 14: TATκ-CDKL5 Protein has the Same Subcellular Localization as the Native CDKL5 and Restores Neurite Development of Hippocampal Neurons Derived from a CDKL5 KO Mouse

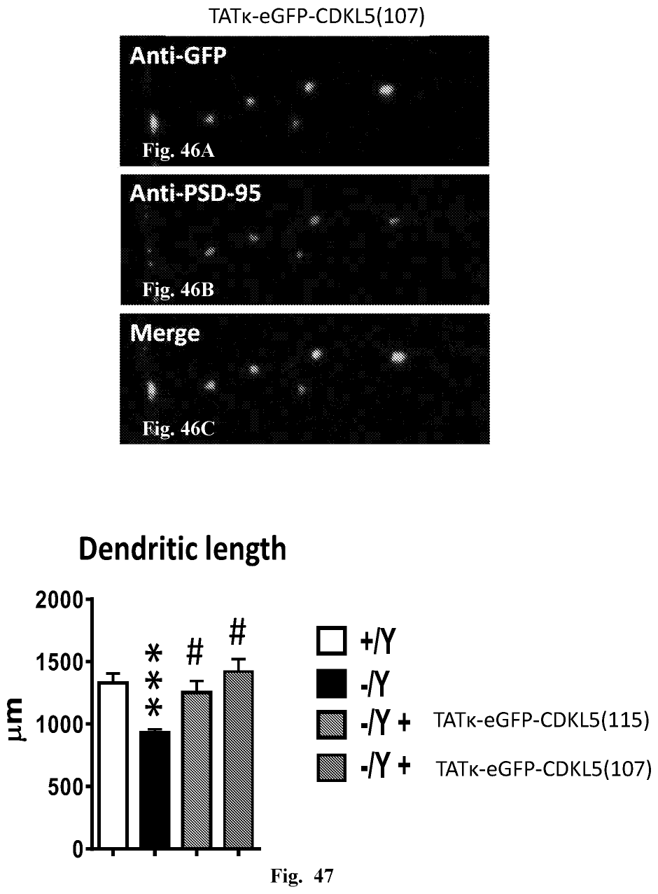

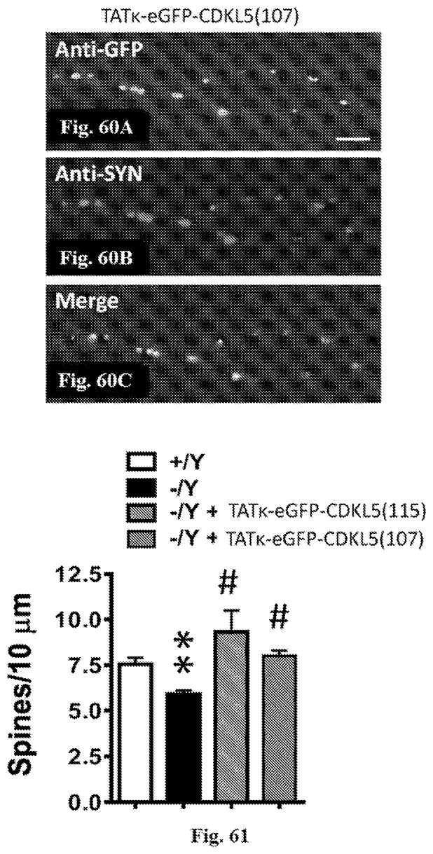

It has been shown that in hippocampal neurons CDKL5 has mainly a cytoplasmic localization with an enrichment at the postsynaptic compartment. We found that in these neurons internalized TATκ-eGFP-CDKL5₁₀₇ localizes mainly in the cytoplasm and, in particular, at the dendritic level it specifically localizes to the dendritic spine (FIGS. 45A-45D). Confocal imagines show TATκ-eGFP-CDKL5 co-localization with presynaptic (synaptophysin; SYN FIGS. 60A-60C) and postsynaptic (PSD-95; FIGS. 46A-46D). This indicates that the exogenous protein localizes at the same subcellular sites of the native CDKL5.

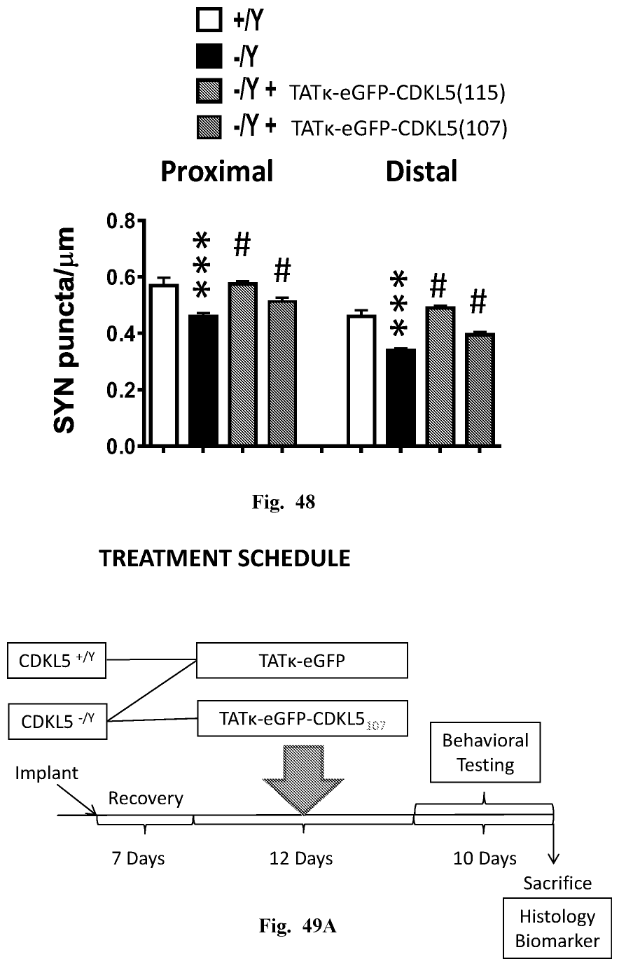

To establish whether TATκ-eGFP-CDKL5₁₀₇ retains CDKL5 physiological activity, hippocampal neuronal cultures from CDKL5 knockout male mice (−/Y) were grown in the presence of TATκ-eGFP-CDKL5₁₀₇ (added to the culture medium) for 8 days. In these neurons an absence of CDKL5 causes a reduction in neuronal maturation, as shown by reduced dendritic length (FIG. 47), number of synaptic connections (FIG. 48) and spine density (FIG. 61). Treatment with TATκ-eGFP-CDKL5 restores neurite development (FIGS. 47-48 and 61), Indicating that the fusion protein has retained the physiological activity of CDKL5.

### Example 15: Effect of TATκ-CDKL5₁₀₇ Fusion Protein on Behavior

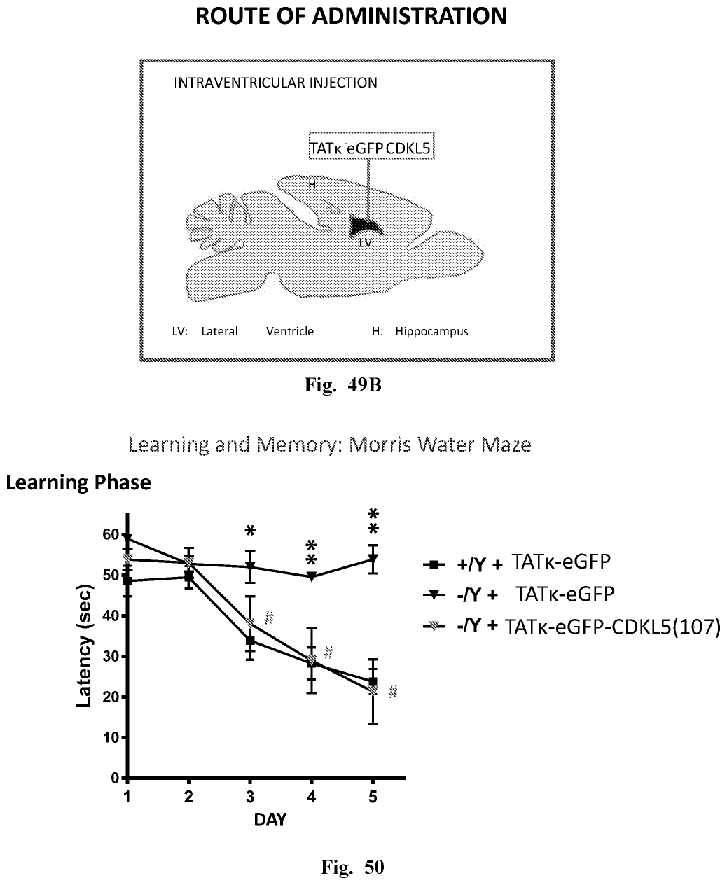

FIGS. 49A-49B show cartoons depicting a treatment schedule and route of administration of the CDKL5 fusion protein for behavioral testing. Male CDKL5 wild-type mice (+/Y) received treatment with TATκ-eGFP (n=6) while CDKL5 KO male mice (−/Y) were treated with TATκ-eGFP (n=6) or TATκ-eGFP-CDKL5₁₀₇ (n=6) as indicated above. Treatment period consisted of a single daily injection (10 μl injection, approximately 50 ng/injection) for 5 consecutive days, followed by a two day rest period and then 5 additional days of a single injection. There was a total of 10 injections which were done in a 12 day period.

FIG. 50 shows a graph demonstrating results from Morris Water Maze testing after receiving the TATκ-eGFP-CDKL5₁₀₇ fusion protein as described in FIGS. 49A-49B. After the treatment period and a two day rest period, mice received Morris Water Maze (MWM) testing. This test measures the ability to find and recall the position of a hidden platform submerged in water. Mice were tested for their ability to learn for 5 days (learning phase) and were subjected to the probe test on day 6 (Page 4). TATκ-eGFP treated wild-type (+/Y) male mice learned to find the platform by the third day, but no significant learning was detected in CDKL5 KO male mice treated with TATκ-eGFP, indicative of a learning deficit. TATκ-eGFP-CDKL5₁₀₇ treated CDKL5 KO male mice began to recover learning ability on day 3 and reached performance similar to WT at days 4 and 5. Values represent mean±SE. * p<0.05, ** p<0.01 as compared to the wild-type condition; #p<0.01 as compared to the TATκ-eGFP treated CDKL5 KO (−/Y) condition (Fisher LSD test after ANOVA).

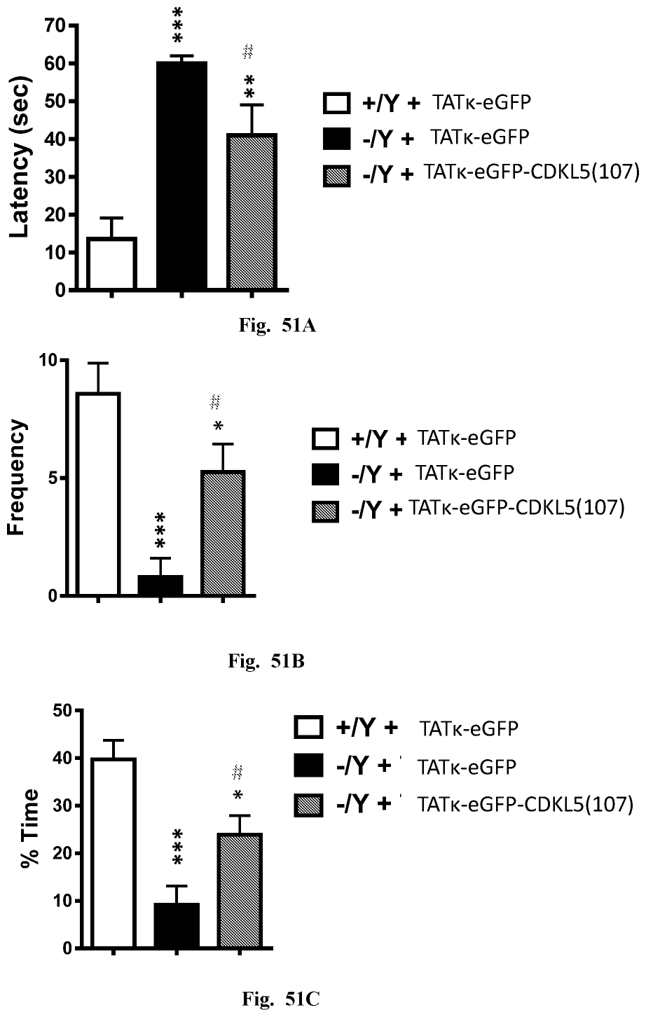

FIGS. 51A-51C show graphs demonstrating spatial memory from measuring (FIG. 51A) latency to enter the former platform quadrant, (FIG. 51B) frequency of entrances into the former platform quadrant, (FIG. 51C) percentage of time spent in the former platform quadrant. Performance in all parameters was severely impaired in TATκ-eGFP treated CDKL5 KO male mice. TATκ-eGFP-CDKL5₁₀₇ treated CDKL5 KO male mice showed statistically significant improvement in all parameters, Figures A, B and C. Values represent mean±SE. * p<0.05, ** p<0.01, *** p<0.001 as compared to the wild-type condition; #p<0.01 as compared to the TATκ-eGFP treated Cdk15 KO −/Y condition (Fisher LSD test after ANOVA).

FIGS. 52A-52B show graphs demonstrating the effect of treatment on learning and memory using a passive avoidance (PA) test. After treatment period and a two day rest period, mice received passive avoidance (PA) testing. The experiment utilized a test cage with two chambers (light and dark). On the first day, animals are placed in the light chamber and instinctively move into the dark chamber where they are conditioned with a single adverse event (foot-shock). FIG. 52A indicates that the latency time to enter the dark chamber was similar for all groups. On the second day (testing period) animals are again placed in the light chamber. Memory of the adverse event was measured by latency time to enter the dark chamber and represented in FIG. 52B. TATκ-eGFP treated CDKL5 KO male (−/Y) mice were severely impaired in this task, as shown by a reduced latency to enter the dark compartment in comparison with wild-type male (+/Y) mice. TATκ-eGFP-CDKL5₁₀₇ treated CDKL5 KO male mice showed similar latency time as compared to wild-type mice (FIG. 52B). These differences were statistically significant in comparison to TATκ-eGFP treated CDKL5 KO male mice. ** p<0.01 as compared to the wild-type male condition; #p<0.01 as compared to the TATκ-eGFP treated CDKL5 KO −/Y condition (Fisher LSD test after ANOVA).

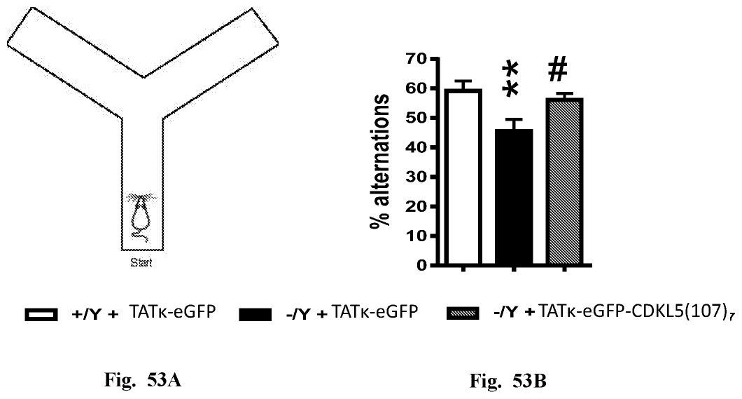

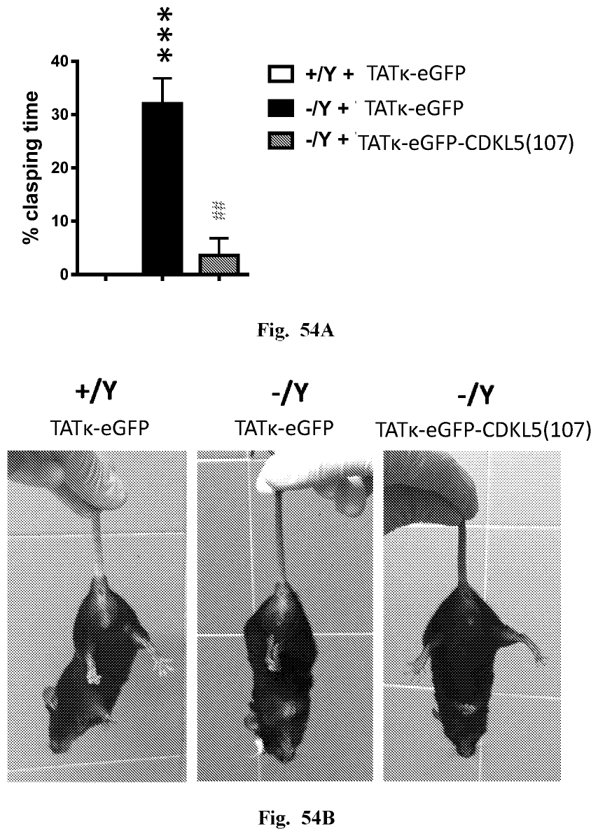

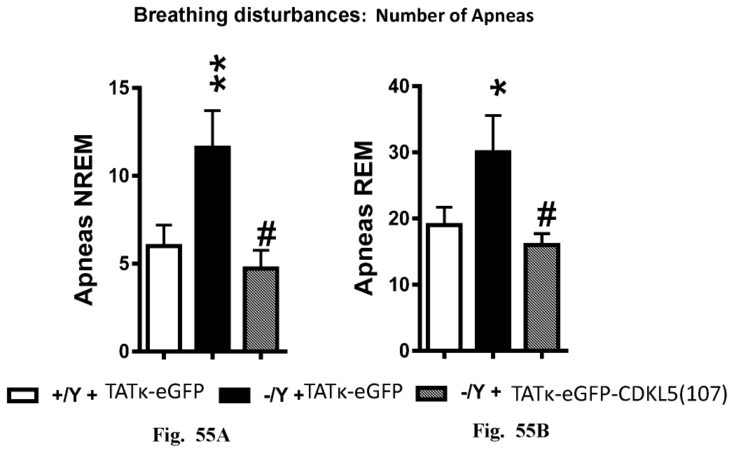

FIGS. 53A-42B show (FIG. 53A) a cartoon of a Y-maze used to evaluate the effect of treatment on learning and memory and (FIG. 53B) a graph demonstrating the results from the Y maze test. After the treatment period and a two day rest period, mice received Y maze testing. Y Maze Spontaneous Alternation was used for measuring the willingness of mice to explore new environments and represents hippocampus-dependent spatial reference memory. Each mouse was placed at the distal part of one arm facing the center of the maze. Each of the three arms was 34 cm×5 cm×10 cm height, angled 120° from the others and made of grey opaque plastic. After introduction into the maze, the animal is allowed to freely explore the three arms for 8 minutes. Over the course of the multiple arm entries, the subject should show a tendency to enter a less recently visited arm. Arm entries were defined by the presence of all four-paws in an arm. The percentage of spontaneous alternations is defined as: (total alternations/total arm entries-2)×100. One alternation is defined as consecutive entries in three different arms. TATκ-eGFP treated CDKL5 KO male (−/Y) mice were impaired in this task, as shown by a reduced percentage of spontaneous alternations in comparison with TATκ-eGFP treated wild-type male (+/Y) mice. CDKL5 KO male mice treated with TATκ-eGFP-CDKL5₁₀₇ showed performance similar to WT treated with TATκ-eGFP. * p<0.05, ** p<0.01 as compared to the wild-type male condition; #p<0.05 as compared to the TATκ-eGFP treated CDKL5 KO male condition (Fisher LSD test after ANOVA).

FIGS. 54A-54B show (FIG. 54A) a graph and (FIG. 54B) an image demonstrating clasping (right mouse) vs. unclasping (left mouse) in a hind limb clasping test used to evaluate the effect of treatment on motor function. After the treatment period, animals were suspended in air by the tail. All animals were suspended for 2 minutes and total time of hind-limb clasping was measured. The figure above reports time of hind-limb clasping as a percentage of the total time suspended. Treatment with TATκ-eGFP-CDKL5₁₀₇ led to a statistically significant reduction in clasping time as compared to TATκ-eGFP treated CDKL5 KO male (−/Y) mice. Values represent mean±SE. *** p<0.001 as compared to the wild-type condition; ##p<0.001 as compared to the TATκ-eGFP treated CDKL5 KO male condition (Fisher LSD test after ANOVA).

FIGS. 55A and 55B show graphs demonstrating breathing disturbances in CDKL5 KO (−/Y) male mice as measured by the number of apneas during non-REM (NREM) (FIG. 55A) and REM (FIG. 55B) sleep. Treatment with TATκ-eGFP-CDKL5₁₀₇ led to a drastic reduction in the number of apneas during non-REM (NREM) (FIG. 55A) and REM (FIG. 55B) sleep in CDKL5 KO male mice.

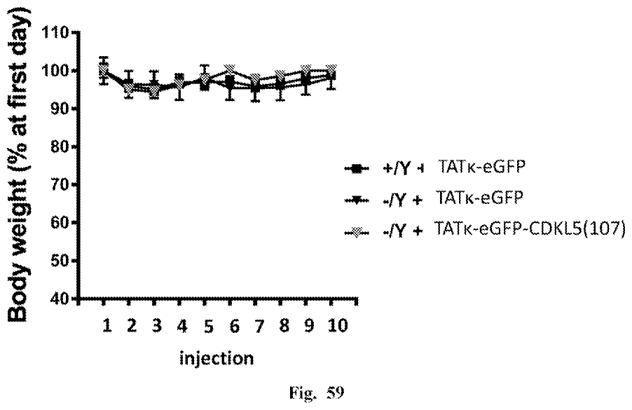

Body weight of male wild-type (+/Y) and CDKL5 KO mice injected for 10 (−/Y) days with TATκ-eGFP-CDKL5₁₀₇ protein was measured and results are demonstrated in FIG. 59. No significant changes in body weight during the injection period were observed, suggesting that there were no side effects caused by TATκ-eGFP-CDKL5 protein administration.

### Example 16: Long Lasting Effect of TATκ-CDKL5₁₀₇ In Vivo Treatment on Neuronal Maturation and Survival

A protein replacement therapy needs to be continued during the whole life span of the patient. With the idea of possibly reducing the frequency of injections in view of a future treatment protocol in humans, we evaluated the persistence of positive effects after treatment cessation.

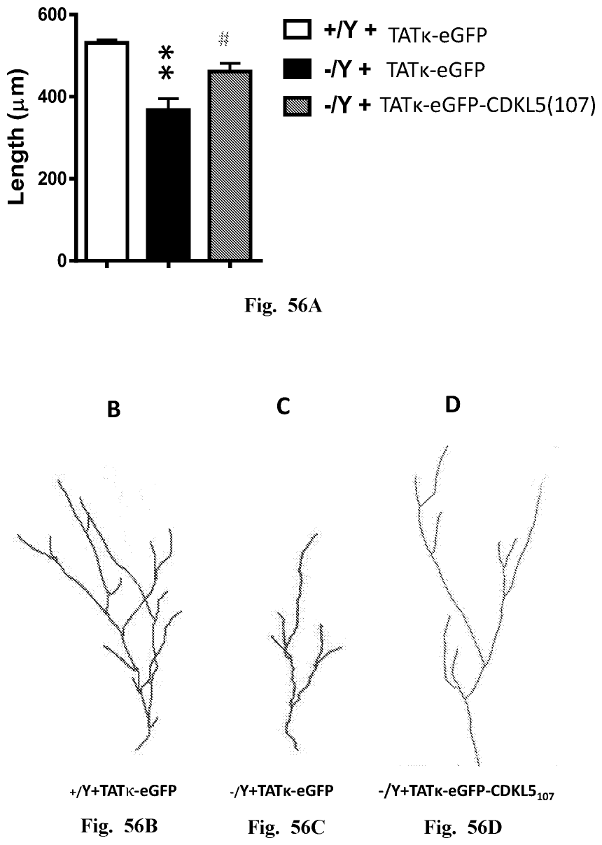

FIGS. 56A-56D show a graph (FIG. 56A) and (FIGS. 56B-56D) reconstructed dendritic trees of newborn granule cells demonstrating the effect of treatment with TATκ-eGFP-CDK5₁₀₇ fusion protein. Twelve days after the completion of the treatment period, granule cell dendritic morphology was analyzed with immunohistochemistry for DCX, a protein present in the cytoplasm during the period of neurite elongation (from one to four weeks after neuron birth). Figure A represents mean total dendritic length of male wild-type (+/Y) (WT) and male CDKL5 KO (−/Y) mice treated with TATκ-eGFP and male CDKL5 KO (−/Y) mice treated with TATκ-eGFP-CDKL5₁₀₇. FIGS. 56B, 56C and 56D show examples of the reconstructed dendritic tree of newborn granule cells of male wild-type (+/Y) mice treated with TATκ-eGFP, male CDKL5 KO (−/Y) mice treated with TATκ-eGFP and male CDKL5 KO (−/Y) mice treated with TATκ-eGFP-CDKL5₁₀₇ respectively. Data indicate treatment with TATκ-eGFP-CDKL5₁₀₇ leads to a morphological change that is durable for at least 12 days from time when dosing was discontinued. Values represent mean±SE. ** p<0.01 as compared to the wild-type condition; #p<0.01 as compared to the TATκ-eGFP treated (−/Y) condition (Bonferroni test after ANOVA).

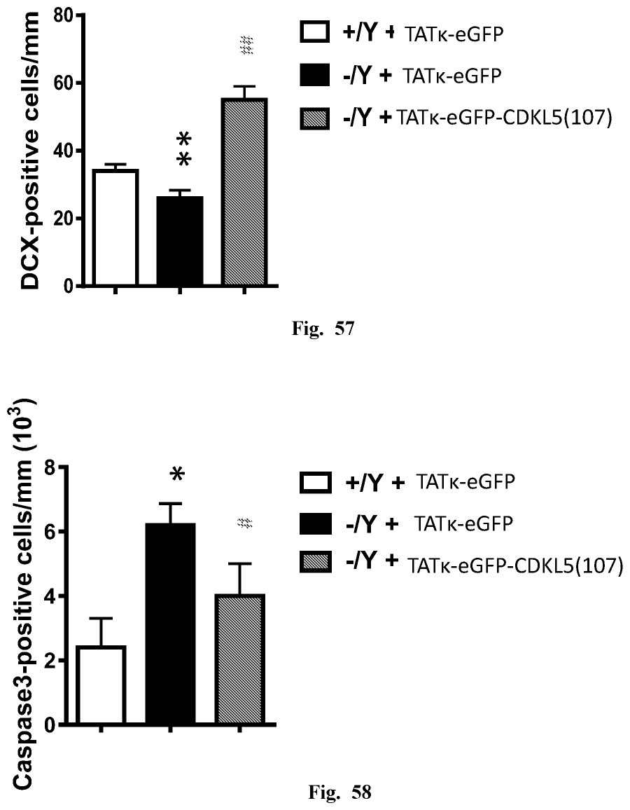

FIG. 57 demonstrates quantification of the number of DCX positive cells in the hippocampus (dentate gyrus) of wild-type (WT) male mice (+/Y), CDKL5 KO male mice (−/Y), and CDKL5 KO male mice treated with TATκ-eGFP-CDKL5₁₀₇. The treatment period consisted of once daily intraventricular injection for 5 days followed by two a day rest period then an additional once daily injection for 5 days. Animals were sacrificed 10 days after the last injection. Data are expressed as number of cells/μm ** p<0.01 as compared to the number of cells/μm in the +/Y+TATκ-eGFP samples; ##p<0.001 as compared to the number of cells/μm in the −/Y+TATκ-eGFP samples (Bonferroni's test after ANOVA). Data suggest that the positive impact of treatment with TATκ-eGFP-CDKL5 on the number of DCX-positive cells is retained 10 days after treatment completion.

FIG. 58 demonstrates quantification of the total number of cleaved Caspase 3 positive cells in the hippocampus (dentate gyrus) of wild-type (WT) male mice (+/Y), CDKL5 KO male mice (−/Y), and CDKL5 KO male mice treated with TATκ-eGFP-CDKL5₁₀₇. The treatment period consisted of a once daily intraventricular injection for 5 days followed by two a day rest period then an additional once daily injection for 5 days. Animals were sacrificed 10 days after the last injection.

### Example 17: Effect of Systemically Administered TATκ-CDKL5₁₀₇ Protein on Neuronal Development and Behavior

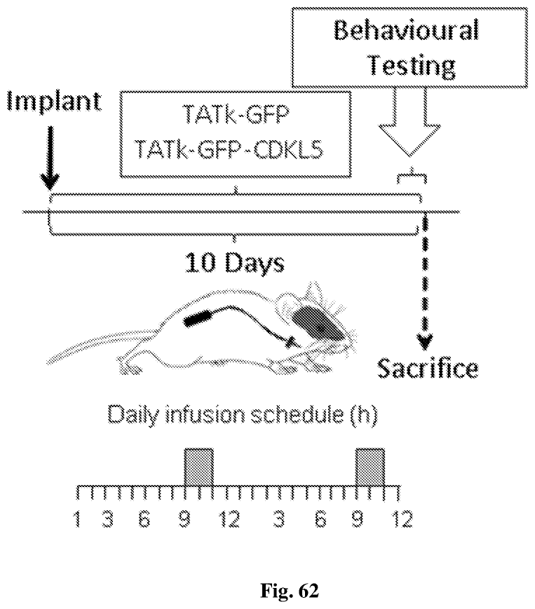

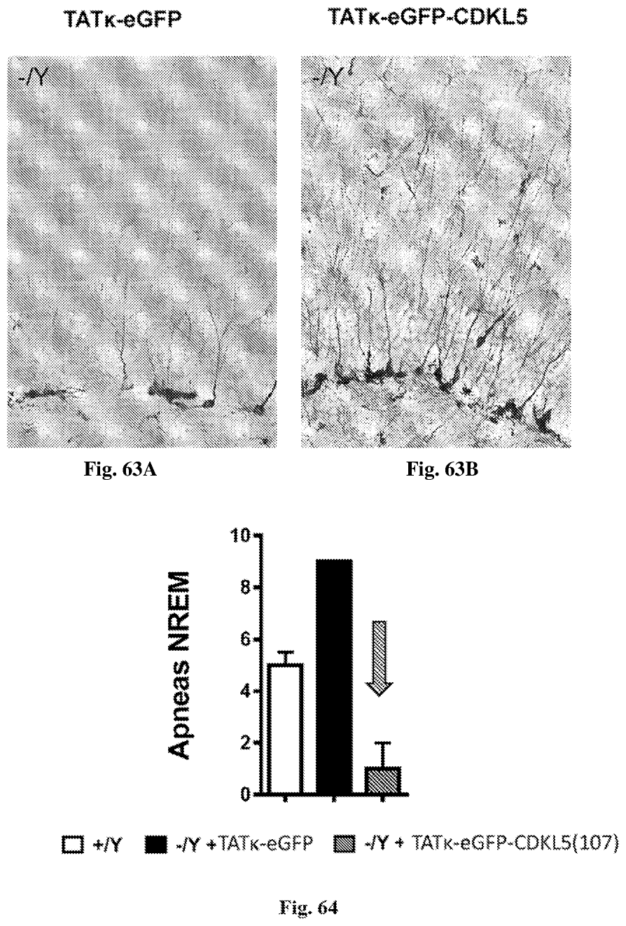

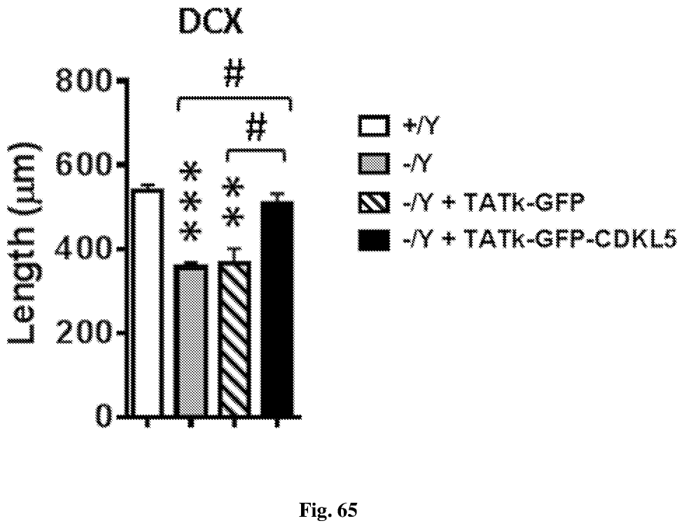

FIG. 62 shows a treatment schedule for systemic administration of the CDKL5 fusion proteins. To mimic a daily human dose administration we used an innovative infusion method which is based on a programmable pump implanted under the skin with a refillable reservoir. The pump was connected to a cannula implanted in the carotid artery. This system allowed us to apply a twice a day infusion protocol (morning and evening) for the duration of 10 days. Male CDKL5 KO (−/Y) mice were infused twice a day with TATκ-eGFP (20 μl+20 μl) or TATκ-eGFP-CDKL5₁₀₇ (20 μl+20 μl) for the duration of 10 days. The two periods of infusion were 9-10 a.m. and 9-10 p.m. This treatment schedule was used to generate the data shown in FIGS. 63A-72. In FIGS. 65-68 and 70-72, values are represented as means±SE. * p<0.05; ** p<0.01; *** p<0.001 as compared to the untreated CDKL5+/Y condition; #p<0.05 as compared to the untreated CDKL5 −/Y samples (Fisher LSD test after ANOVA).

As shown in FIGS. 17A-17F, the CDKL5 fusion protein 115 isoform can cross the blood-brain barrier when administered systemically. FIGS. 63A-63B demonstrate the effect of systemic treatment with the TATκ-eGFP-CDKL5₁₀₇ on newborn granule cell maturation. One hour after the last injection animals were sacrificed, and the dendritic morphology of newborn hippocampal granule cells was analyzed with immunohistochemistry for DCX. FIGS. 63A-63B demonstrate that DCX positive neurons of treated male CDKL5 knockout mice had longer processes than those of untreated male CDKL5 KO mice. FIG. 64 shows that apneas during sleep are drastically reduced in the mice treated with TATκ-eGFP-CDKL5₁₀₇, indicating a positive effect of the treatment.

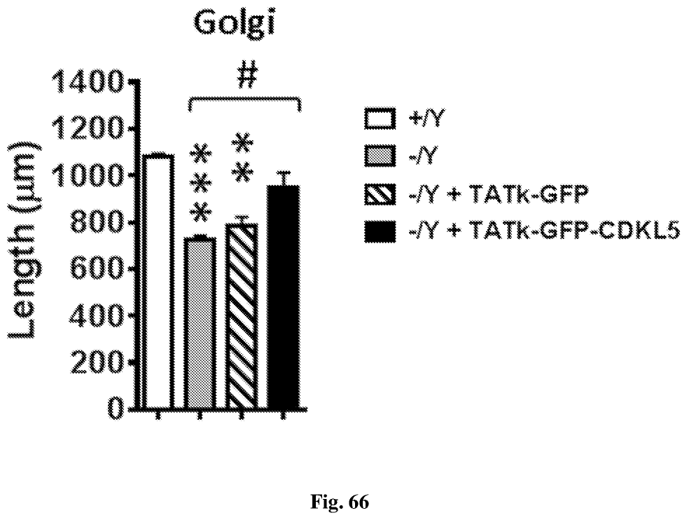

FIG. 65 shows the total dendritic length of newborn (doublecortin-positive) granule cells of untreated CDKL +/Y (n=5) and CDKL5 −/Y (n=5) mice, and CDKL5 −/Y mice treated with TATκ-eGFP (n=6) or TATκ-eGFP-CDKL5₁₀₇ (n=6). FIG. 66 shows the total dendritic length of Golgi-stained granule cells of untreated CDKL5+/Y (n=5) and CDKL5 −/Y (n=5) mice, and CDKL5 −/Y mice treated with TATκ-eGFP (n=4) or TATκ-eGFP-CDKL5₁₀₇ (n=5). As can be seen from FIGS. 65 and 66, systemic administration of the CDKL5 fusion protein significantly increased dendritic length compared to the untreated CDKL5+/Y mice.

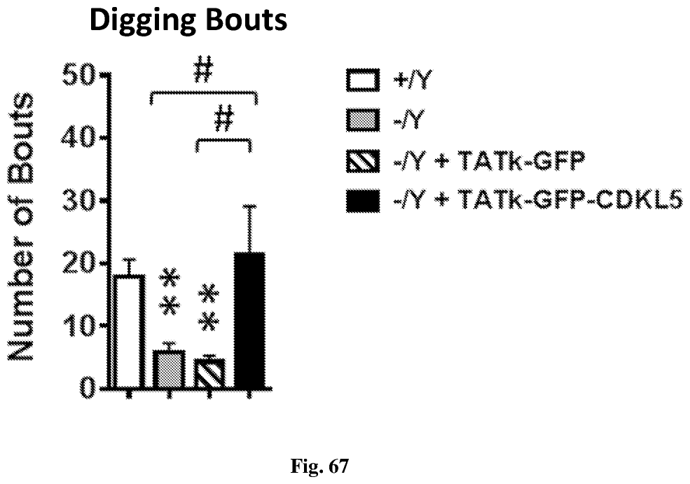

FIG. 67 shows the number of digging bouts of untreated CDKL5+/Y (n=24) and CDKL5 −/Y (n=11) mice, and CDKL5 −/Y mice treated with TATκ-eGFP (n=6) or TATκ-eGFP-CDKL5₁₀₇ (n=5). As can be seen from FIG. 67, systemic administration of the CDKL5 fusion protein significantly increased the number of digging bouts compared to the untreated CDKL5+/Y mice.

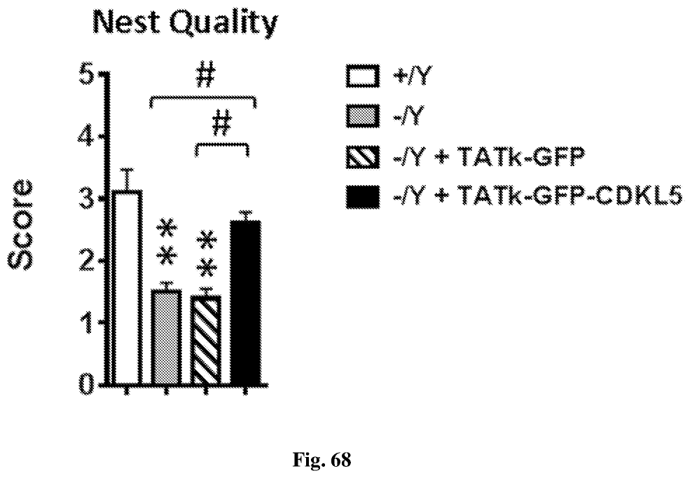

FIG. 68 shows the nest quality of untreated CDKL5+/Y (n=6) and CDKL5 −/Y (n=20) mice, and CDKL5 −/Y mice treated with TATκ-eGFP (n=6) or TATκ-eGFP-CDKL5₁₀₇ (n=5). As can be seen from FIG. 68, systemic administration of the CDKL5 fusion protein significantly increased the nest quality compared to the untreated CDKL5+/Y mice.

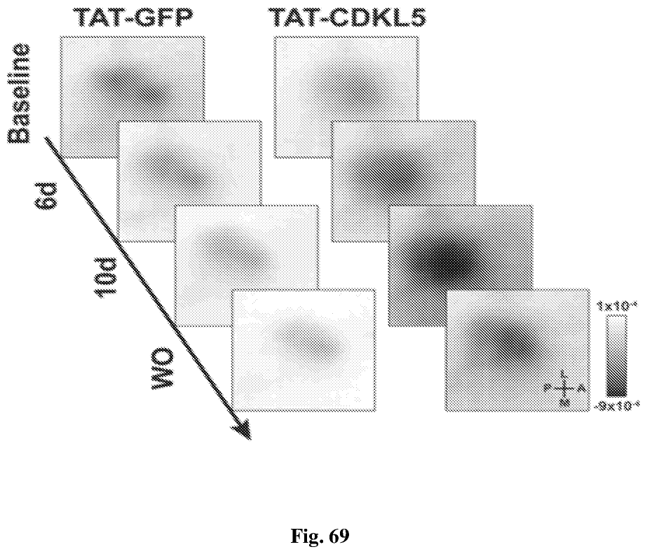

FIG. 69 shows representative images of neural activity in the visual cortex collected at different time points in one CDKL5 −/Y mouse treated with either TATκ-eGFP or TATκ-eGFP-CDKL5₁₀₇. In FIG. 69, a darker image shows a higher level of neural activity and a lighter image shows a lower level of neural activity. As can be seen from FIG. 69, the mouse treated with TATκ-eGFP had very little neural activity in the visual cortex, whereas the mouse treated with TATκ-eGFP-CDKL5₁₀₇ regained visual activity throughout the 10-day treatment period and retained visual activity during the washout period.

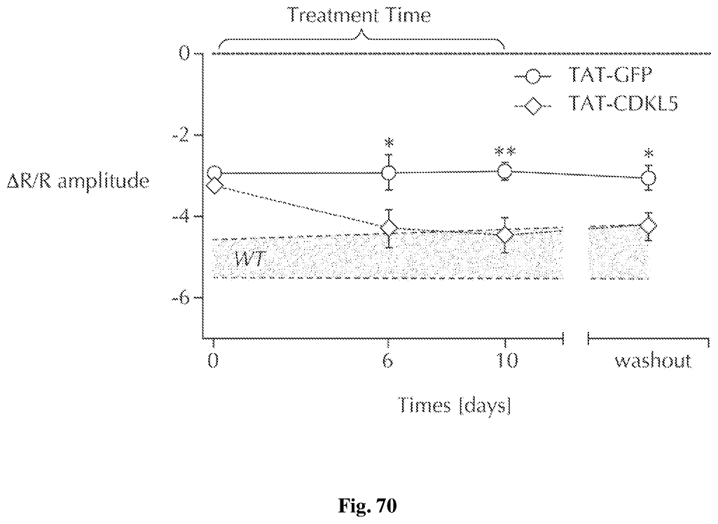

FIG. 70 shows the mean amplitude of visually evoked responses measured before and after 6 and 10 days of treatment in CDKL5 −/Y mice treated with TATκ-eGFP or TATκ-eGFP-CDKL5₁₀₇. The persistence of the effect was evaluated with an additional measurement 6-10 days after treatment cessation (washout). As a reference, the 95% confidence interval of untreated wild-type response amplitude over time is shown in the patterned area. Error bars represent standard error of the mean. Two-way ANOVA (repeated measures for the factor time) revealed a time X treatment interaction p<0.05; post-hoc Holm-Sidak's multiple comparisons test: * p<0.05, **p<0.01. As can be seen from FIG. 70, systemic administration of the CDKL5 fusion protein for 6 and 10 days significantly decreased the mean amplitude of visually evoked responses compared to the CDKL5+/Y mice treated with TATκ-eGFP. This trend continued even after the cessation of treatment.

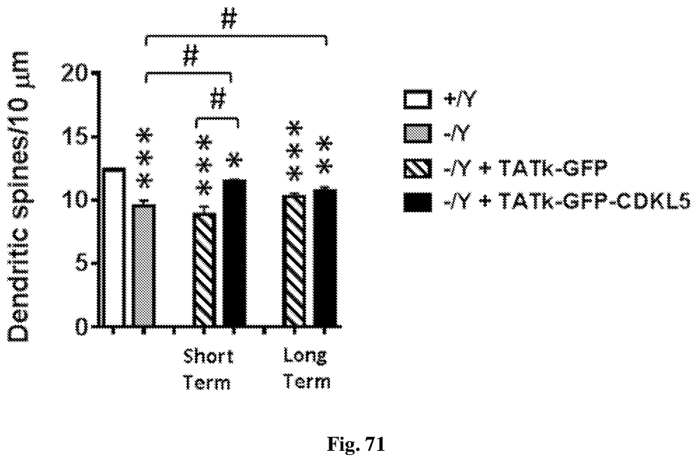

FIG. 71 shows the dendritic spine density of the primary visual cortex (V1) pyramidal neurons (layer 2/3) from untreated CDKL5+/Y (n=5) and CDKL5 −/Y (n=5) mice and CDKL5 −/Y mice treated with TATκ-eGFP (n=4) or TATκ-eGFP-CDKL5₁₀₇ (n=5) and sacrificed at the end of treatment (short term), or with TATκ-eGFP (n=3) or TATκ-eGFP-CDKL5₁₀₇ (n=3) and sacrificed 10 days after treatment cessation (long term). As can be seen from FIG. 71, systemic administration of the CDKL5 fusion protein significantly increased the dendritic spine density compared to the untreated CDKL5+/Y mice. This trend continued even after the cessation of treatment.

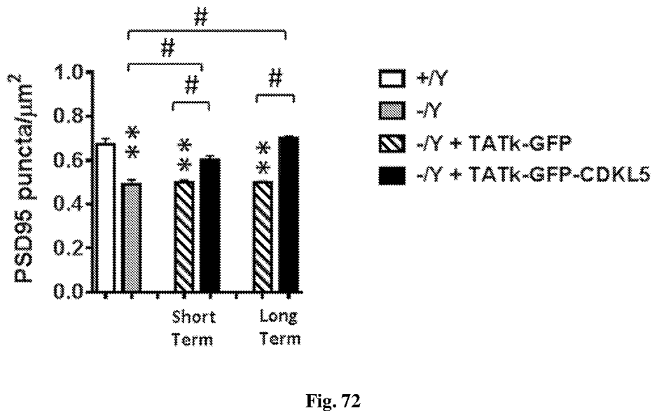

FIG. 72 shows the number of fluorescent puncta per μm² exhibiting PSD-95 immunoreactivity in the primary visual cortex (V1) of untreated CDKL5+/Y (n=3) and CDKL5 −/Y (n=3) mice, and CDKL5 −/Y mice treated with TATκ-eGFP (n=4) or TATκ-eGFP-CDKL5₁₀₇ (n=6) and sacrificed at the end of treatment (short term), or with TATκ-eGFP (n=4) or TATκ-eGFP-CDKL5₁₀₇ (n=4) and sacrificed 10 days after treatment cessation (long term). As can be seen from FIG. 72, systemic administration of the CDKL5 fusion protein significantly increased the number of PSD-95 fluorescent puncta per μm² compared to the untreated CDKL5+/Y mice. This trend continued even after the cessation of treatment.

These data support the systemic (e.g., intravenous) administration of CDKL5 fusion proteins for the treatment of CDKL5 deficiencies, increasing dendrite length, increasing neural activity in the visual cortex, and improving behavior.

SEQUENCE LISTING
CDKL5 115 isoform cDNA (lacking the ATG start codon for the
initiator methionine)
SEQ ID NO: 1
aagattcctaacattggtaatgtgatgaataaatttgagatccttggggttgtaggtgaagga
gcctatggagttgtacttaaatgcagacacaaggaaacacatgaaattgtggcgatcaagaaa
ttcaaggacagtgaagaaaatgaagaagtcaaagaaacgactttacgagagcttaaaatgctt
cggactctcaagcaggaaaacattgtggagttgaaggaagcatttcgtcggaggggaaagttg
tacttggtgtttgagtatgttgaaaaaaatatgctcgaattgctggaagaaatgccaaatgga
gttccacctgagaaagtaaaaagctacatctatcagctaatcaaggctattcactggtgccat
aagaatgatattgtccatcgagatataaaaccagaaaatctcttaatcagccacaatgatgtc
ctaaaactgtgtgactttggttttgctcgtaatctgtcagaaggcaataatgctaattacaca
gagtacgttgccaccagatggtatcggtccccagaactcttacttggcgctccctatggaaag
tccgtggacatgtggtcggtgggctgtattcttggggagcttagcgatggacagcctttattt
cctggagaaagtgaaattgaccaactttttactattcagaaggtgctaggaccacttccatct
gagcagatgaagcttttctacagtaatcctcgcttccatgggctccggtttccagctgttaac
catcctcagtccttggaaagaagataccttggaattttgaatagtgttctacttgacctaatg
aagaatttactgaagttggacccagctgacagatacttgacagaacagtgtttgaatcaccct
acatttcaaacccagagacttctggatcgttctccttcaaggtcagcaaaaagaaaaccttac
catgtggaaagcagcacattgtctaatagaaaccaagccggcaaaagtactgctttgcagtct
caccacagatctaacagcaaggacatccagaacctgagtgtaggcctgccccgggctgacgaa
ggtctccctgccaatgaaagcttcctaaatggaaaccttgctggagctagtcttagtccactg
cacaccaaaacctaccaagcaagcagccagcctgggtctaccagcaaagatctcaccaacaac
aacataccacaccttcttagcccaaaagaagccaagtcaaaaacagagtttgattttaatatt
gacccaaagccttcagaaggcccagggacaaagtacctcaagtcaaacagcagatctcagcag
aaccgccactcattcatggaaagctctcaaagcaaagctgggacactgcagcccaatgaaaag
cagagtcggcatagctatattgacacaattccccagtcctctaggagtccctcctacaggacc
aaggccaaaagccatggggcactgagtgactccaagtctgtgagcaacctttctgaagccagg
gcccaaattgcggagcccagtaccagtaggtacttcccatctagctgcttagacttgaattct
cccaccagcccaacccccaccagacacagtgacacgagaactttgctcagcccttctggaaga
aataaccgaaatgagggaacgctggactcacgtcgaaccacaaccagacattctaagacgatg
gaggaattgaagctgccggagcacatggacagtagccattcccattcactgtctgcacctcac
gaatctttttcttatggactgggctacaccagccccttttcttcccagcaacgtcctcatagg
cattctatgtatgtgacccgtgacaaagtgagagccaagggcttggatggaagcttgagcata
gggcaagggatggcagctagagccaacagcctgcaactcttgtcaccccagcctggagaacag
ctccctccagagatgactgtggcaagatcttcggtcaaagagacctccagagaaggcacctct
tccttccatacacgccagaagtctgagggtggagtgtatcatgacccacactctgatgatggc
acagcccccaaagaaaatagacacctatacaatgatcctgtgccaaggagagttggtagcttt
tacagagtgccatctccacgtccagacaattctttccatgaaaataatgtgtcaactagagtt
tcttctctaccatcagagagcagttctggaaccaaccactcaaaaagacaaccagcattcgat
ccatggaaaagtcctgaaaatattagtcattcagagcaactcaaggaaaaagagaagcaagga
tttttcaggtcaatgaaaaagaaaaagaagaaatctcaaacagtacccaattccgacagccct
gatcttctgacgttgcagaaatccattcattctgctagcactccaagcagcagaccaaaggag
tggcgccccgagaagatctcagatctgcagacccaaagccagccattaaaatcactgcgcaag
ttgttacatctctcttcggcctcaaatcacccggcttcctcagatccccgcttccagccctta
acagctcaacaaaccaaaaattccttctcagaaattcggattcaccccctgagccaggcctct
ggcgggagcagcaacatccggcaggaacccgcaccgaagggcaggccagccctccagctgcca
gacggtggatgtgatggcagaagacagagacaccattctggaccccaagatagacgcttcatg
ttaaggacgacagaacaacaaggagaatacttctgctgtggtgacccaaagaagcctcacact
ccgtgcgtcccaaaccgagcccttcatcgtccaatctccagtcctgctccctatccagtactc
caggtccgaggcacttccatgtgcccgacactccaggtccgaggcactgatgctttcagctgc
ccaacccagcaatccgggttctctttcttcgtgagacacgttatgagggaagccctgattcac
agggcccaggtaaaccaagctgcgctcctgacataccatgagaatgcggcactgacgggcaag
CDKL5 isoform 115 polypeptide (lacking the initiator methionine)
SEQ ID NO: 2
KIPNIGNVMNKFEILGVVGEGAYGVVLKCRHKETHEIVAIKKFKDSEENEEVKETTLRELKML
RTLKQENIVELKEAFRRRGKLYLVFEYVEKNMLELLEEMPNGVPPEKVKSYIYQLIKAIHWCH
KNDIVHRDIKPENLLISHNDVLKLCDFGFARNLSEGNNANYTEYVATRWYRSPELLLGAPYGK
SVDMWSVGCILGELSDGQPLFPGESEIDQLFTIQKVLGPLPSEQMKLFYSNPRFHGLRFPAVN
HPQSLERRYLGILNSVLLDLMKNLLKLDPADRYLTEQCLNHPTFQTQRLLDRSPSRSAKRKPY
HVESSTLSNRNQAGKSTALQSHHRSNSKDIQNLSVGLPRADEGLPANESFLNGNLAGASLSPL
HTKTYQASSQPGSTSKDLTNNNIPHLLSPKEAKSKTEFDFNIDPKPSEGPGTKYLKSNSRSQQ
NRHSFMESSQSKAGTLQPNEKQSRHSYIDTIPQSSRSPSYRTKAKSHGALSDSKSVSNLSEAR
AQIAEPSTSRYFPSSCLDLNSPTSPTPTRHSDTRTLLSPSGRNNRNEGTLDSRRTTTRHSKTM
EELKLPEHMDSSHSHSLSAPHESFSYGLGYTSPFSSQQRPHRHSMYVTRDKVRAKGLDGSLSI
GQGMAARANSLQLLSPQPGEQLPPEMTVARSSVKETSREGTSSFHTRQKSEGGVYHDPHSDDG
TAPKENRHLYNDPVPRRVGSFYRVPSPRPDNSFHENNVSTRVSSLPSESSSGTNHSKRQPAFD
PWKSPENISHSEQLKEKEKQGFFRSMKKKKKKSQTVPNSDSPDLLTLQKSIHSASTPSSRPKE
WRPEKISDLQTQSQPLKSLRKLLHLSSASNHPASSDPRFQPLTAQQTKNSFSEIRIHPLSQAS
GGSSNIRQEPAPKGRPALQLPDGGCDGRRQRHHSGPQDRRFMLRTTEQQGEYFCCGDPKKPHT
PCVPNRALHRPISSPAPYPVLQVRGTSMCPTLQVRGTDAFSCPTQQSGFSFFVRHVMREALIH
RAQVNQAALLTYHENAALTGK
TATκ polynucleotide sequence
SEQ ID NO: 3
tacgccagaaaggccgccaggcaggccagggca
TATκ polypeptide sequence
SEQ ID NO: 4
YARKAARQARA
Igκ-chain leader polynucleotide sequence
SEQ ID NO: 5
atggagacagacacactcctgctatgggtactgctgctctgggttccaggttccactggt
Igκchain leader polypeptide
SEQ ID NO: 6
METDTLLLWVLLLWVPGSTG
CDKL5 115 isoform fusion protein (Igκ-TATκ-CDKL5(115)-MYC-
HIS) polynucleotide
SEQ ID NO: 7
atggagacagacacactcctgctatgggtactgctgctctgggttccaggttccactggtgac
gcggcccagccggccaggcgcgcgcgccgtacgaagcttgcggcctacgccagaaaggccgcc
aggcaggccagggcaccggtgaagattcctaacattggtaatgtgatgaataaatttgagatc
cttggggttgtaggtgaaggagcctatggagttgtacttaaatgcagacacaaggaaacacat
gaaattgtggcgatcaagaaattcaaggacagtgaagaaaatgaagaagtcaaagaaacgact
ttacgagagcttaaaatgcttcggactctcaagcaggaaaacattgtggagttgaaggaagca
tttcgtcggaggggaaagttgtacttggtgtttgagtatgttgaaaaaaatatgctcgaattg
ctggaagaaatgccaaatggagttccacctgagaaagtaaaaagctacatctatcagctaatc
aaggctattcactggtgccataagaatgatattgtccatcgagatataaaaccagaaaatctc
ttaatcagccacaatgatgtcctaaaactgtgtgactttggttttgctcgtaatctgtcagaa
ggcaataatgctaattacacagagtacgttgccaccagatggtatcggtccccagaactctta
cttggcgctccctatggaaagtccgtggacatgtggtcggtgggctgtattcttggggagctt
agcgatggacagcctttatttcctggagaaagtgaaattgaccaactttttactattcagaag
gtgctaggaccacttccatctgagcagatgaagcttttctacagtaatcctcgcttccatggg
ctccggtttccagctgttaaccatcctcagtccttggaaagaagataccttggaattttgaat
agtgttctacttgac
ctaatgaagaatttactgaagttggacccagctgacagatacttgacagaacagtgtttgaat
caccctacatttcaaacccagagacttctggatcgttctccttcaaggtcagcaaaaagaaaa
ccttaccatgtggaaagcagcacattgtctaatagaaaccaagccggcaaaagtactgctttg
cagtctcaccacagatctaacagcaaggacatccagaacctgagtgtaggcctgccccgggct
gacgaaggtctccctgccaatgaaagcttcctaaatggaaaccttgctggagctagtcttagt
ccactgcacaccaaaacctaccaagcaagcagccagcctgggtctaccagcaaagatctcacc
aacaacaacataccacaccttcttagcccaaaagaagccaagtcaaaaacagagtttgatttt
aatattgacccaaagccttcagaaggcccagggacaaagtacctcaagtcaaacagcagatct
cagcagaaccgccactcattcatggaaagctctcaaagcaaagctgggacactgcagcccaat
gaaaagcagagtcggcatagctatattgacacaattccccagtcctctaggagtccctcctac
aggaccaaggccaaaagccatggggcactgagtgactccaagtctgtgagcaacctttctgaa
gccagggcccaaattgcggagcccagtaccagtaggtacttcccatctagctgcttagacttg
aattctcccaccagcccaacccccaccagacacagtgacacgagaactttgctcagcccttct
ggaagaaataaccgaaatgagggaacgctggactcacgtcgaaccacaaccagacattctaag
acgatggaggaattgaagctgccggagcacatggacagtagccattcccattcactgtctgca
cctcacgaatctttt
tcttatggactgggctacaccagccccttttcttcccagcaacgtcctcataggcattctatg
tatgtgacccgtgacaaagtgagagccaagggcttggatggaagcttgagcatagggcaaggg
atggcagctagagccaacagcctgcaactcttgtcaccccagcctggagaacagctccctcca
gagatgactgtggcaagatcttcggtcaaagagacctccagagaaggcacctcttccttccat
acacgccagaagtctgagggtggagtgtatcatgacccacactctgatgatggcacagccccc
aaagaaaatagacacctatacaatgatcctgtgccaaggagagttggtagcttttacagagtg
ccatctccacgtccagacaattctttccatgaaaataatgtgtcaactagagtttcttctcta
ccatcagagagcagttctggaaccaaccactcaaaaagacaaccagcattcgatccatggaaa
agtcctgaaaatattagtcattcagagcaactcaaggaaaaagagaagcaaggatttttcagg
tcaatgaaaaagaaaaagaagaaatctcaaacagtacccaattccgacagccctgatcttctg
acgttgcagaaatccattcattctgctagcactccaagcagcagaccaaaggagtggcgcccc
gagaagatctcagatctgcagacccaaagccagccattaaaatcactgcgcaagttgttacat
ctctcttcggcctcaaatcacccggcttcctcagatccccgcttccagcccttaacagctcaa
caaaccaaaaattccttctcagaaattcggattcaccccctgagccaggcctctggcgggagc
agcaacatccggcaggaacccgcaccgaagggcaggccagccctccagctgccagacggtgga
tgtgatggcagaaga
cagagacaccattctggaccccaagatagacgcttcatgttaaggacgacagaacaacaagga
gaatacttctgctgtggtgacccaaagaagcctcacactccgtgcgtcccaaaccgagccctt
catcgtccaatctccagtcctgctccctatccagtactccaggtccgaggcacttccatgtgc
ccgacactccaggtccgaggcactgatgctttcagctgcccaacccagcaatccgggttctct
ttcttcgtgagacacgttatgagggaagccctgattcacagggcccaggtaaaccaagctgcg
ctcctgacataccatgagaatgcggcactgacgggcaagtccgctcgaggagggcccgaacaa
aaactcatctcagaagaggatctgaatagcgccgtcgaccatcatcatcatcatcattga
CDKL5 115 isoform fusion protein (Igκ-TATκ-CDKL5(115)-MYC-
HIS) polypeptide
SEQ ID NO: 8
METDTLLLWVLLLWVPGSTGDAAQPARRARRTKLAAYARKAARQARAPVKIPNIGNVMNKFEI
LGVVGEGAYGVVLKCRHKETHEIVAIKKFKDSEENEEVKETTLRELKMLRTLKQENIVELKEA
FRRRGKLYLVFEYVEKNMLELLEEMPNGVPPEKVKSYIYQLIKAIHWCHKNDIVHRDIKPENL
LISHNDVLKLCDFGFARNLSEGNNANYTEYVATRWYRSPELLLGAPYGKSVDMWSVGCILGEL
SDGQPLFPGESEIDQLFTIQKVLGPLPSEQMKLFYSNPRFHGLRFPAVNHPQSLERRYLGILN
SVLLDLMKNLLKLDPADRYLTEQCLNHPTFQTQRLLDRSPSRSAKRKPYHVESSTLSNRNQAG
KSTALQSHHRSNSKDIQNLSVGLPRADEGLPANESFLNGNLAGASLSPLHTKTYQASSQPGST
SKDLTNNNIPHLLSPKEAKSKTEFDFNIDPKPSEGPGTKYLKSNSRSQQNRHSFMESSQSKAG
TLQPNEKQSRHSYIDTIPQSSRSPSYRTKAKSHGALSDSKSVSNLSEARAQIAEPSTSRYFPS
SCLDLNSPTSPTPTRHSDTRTLLSPSGRNNRNEGTLDSRRTTTRHSKTMEELKLPEHMDSSHS
HSLSAPHESFSYGLGYTSPFSSQQRPHRHSMYVTRDKVRAKGLDGSLSIGQGMAARANSLQLL
SPQPGEQLPPEMTVARSSVKETSREGTSSFHTRQKSEGGVYHDPHSDDGTAPKENRHLYNDPV
PRRVGSFYRVPSPRPDNSFHENNVSTRVSSLPSESSSGTNHSKRQPAFDPWKSPENISHSEQL
KEKEKQGFFRSMKKKKKKSQTVPNSDSPDLLTLQKSIHSASTPSSRPKEWRPEKISDLQTQSQ
PLKSLRKLLHLSSASNHPASSDPRFQPLTAQQTKNSFSEIRIHPLSQASGGSSNIRQEPAPKG
RPALQLPDGGCDGRRQRHHSGPQDRRFMLRTTEQQGEYFCCGDPKKPHTPCVPNRALHRPISS
PAPYPVLQVRGTSMCPTLQVRGTDAFSCPTQQSGFSFFVRHVMREALIHRAQVNQAALLTYHE
NAALTGKSARGGPEQKLISEEDLNSAVDHHHHHH
CDKL5 115 isoform fusion protein (Igk-TATκ-eGFP-CDKL5(115)-MYC-
HIS) polynucleotide. Underline indicates codon for the initiator
methionine.
SEQ ID NO: 9
gctagccaccatggagacagacacactcctgctatgggtactgctgctctgggttccaggttc
cactggtgacgcggcccagccggccaggcgcgcgcgccgtacgaagcttgcggcctacgccag
aaaggccgccaggcaggccagggcaccggtcgccaccatggtgagcaagggcgaggagctgtt
caccggggtggtgcccatcctggtcgagctggacggcgacgtaaacggccacaagttcagcgt
gtccggcgagggcgagggcgatgccacctacggcaagctgaccctgaagttcatctgcaccac
cggcaagctgcccgtgccctggcccaccctcgtgaccaccctgacctacggcgtgcagtgctt
cagccgctaccccgaccacatgaagcagcacgacttcttcaagtccgccatgcccgaaggcta
cgtccaggagcgcaccatcttcttcaaggacgacggcaactacaagacccgcgccgaggtgaa
gttcgagggcgacaccctggtgaaccgcatcgagctgaagggcatcgacttcaaggaggacgg
caacatcctggggcacaagctggagtacaactacaacagccacaacgtctatatcatggccga
caagcagaagaacggcatcaaggtgaacttcaagatccgccacaacatcgaggacggcagcgt
gcagctcgccgaccactaccagcagaacacccccatcggcgacggccccgtgctgctgcccga
caaccactacctgagcacccagtccgccctgagcaaagaccccaacgagaagcgcgatcacat
ggtcctgctggagttcgtgaccgccgccgggatcactctcggcatggacgagctgtacaagtc
cggactcagatctcgagcgaagattcctaacattggtaatgtgatgaataaatttgagatcct
tggggttgtaggtgaaggagcctatggagttgtacttaaatgcagacacaaggaaacacatga
aattgtggcgatcaagaaattcaaggacagtgaagaaaatgaagaagtcaaagaaacgacttt
acgagagcttaaaatgcttcggactctcaagcaggaaaacattgtggagttgaaggaagcatt
tcgtcggaggggaaagttgtacttggtgtttgagtatgttgaaaaaaatatgctcgaattgct
ggaagaaatgccaaatggagttccacctgagaaagtaaaaagctacatctatcagctaatcaa
ggctattcactggtgccataagaatgatattgtccatcgagatataaaaccagaaaatctctt
aatcagccacaatgatgtcctaaaactgtgtgactttggttttgctcgtaatctgtcagaagg
caataatgctaattacacagagtacgttgccaccagatggtatcggtccccagaactcttact
tggcgctccctatggaaagtccgtggacatgtggtcggtgggctgtattcttggggagcttag
cgatggacagcctttatttcctggagaaagtgaaattgaccaactttttactattcagaaggt
gctaggaccacttccatctgagcagatgaagcttttctacagtaatcctcgcttccatgggct
ccggtttccagctgttaaccatcctcagtccttggaaagaagataccttggaattttgaatag
tgttctacttgacctaatgaagaatttactgaagttggacccagctgacagatacttgacaga
acagtgtttgaatcaccctacatttcaaacccagagacttctggatcgttctccttcaaggtc
agcaaaaagaaaaccttaccatgtggaaagcagcacattgtctaatagaaaccaagccggcaa
aagtactgctttgcagtctcaccacagatctaacagcaaggacatccagaacctgagtgtagg
cctgccccgggctgacgaaggtctccctgccaatgaaagcttcctaaatggaaaccttgctgg
agctagtcttagtccactgcacaccaaaacctaccaagcaagcagccagcctgggtctaccag
caaagatctcaccaacaacaacataccacaccttcttagcccaaaagaagccaagtcaaaaac
agagtttgattttaatattgacccaaagccttcagaaggcccagggacaaagtacctcaagtc
aaacagcagatctcagcagaaccgccactcattcatggaaagctctcaaagcaaagctgggac
actgcagcccaatgaaaagcagagtcggcatagctatattgacacaattccccagtcctctag
gagtccctcctacaggaccaaggccaaaagccatggggcactgagtgactccaagtctgtgag
caacctttctgaagccagggcccaaattgcggagcccagtaccagtaggtacttcccatctag
ctgcttagacttgaattctcccaccagcccaacccccaccagacacagtgacacgagaacttt
gctcagcccttctggaagaaataaccgaaatgagggaacgctggactcacgtcgaaccacaac
cagacattctaagacgatggaggaattgaagctgccggagcacatggacagtagccattccca
ttcactgtctgcacctcacgaatctttttcttatggactgggctacaccagccccttttcttc
ccagcaacgtcctcataggcattctatgtatgtgacccgtgacaaagtgagagccaagggctt
ggatggaagcttgagcatagggcaagggatggcagctagagccaacagcctgcaactcttgtc
accccagcctggagaacagctccctccagagatgactgtggcaagatcttcggtcaaagagac
ctccagagaaggcacctcttccttccatacacgccagaagtctgagggtggagtgtatcatga
cccacactctgatgatggcacagcccccaaagaaaatagacacctatacaatgatcctgtgcc
aaggagagttggtagcttttacagagtgccatctccacgtccagacaattctttccatgaaaa
taatgtgtcaactagagtttcttctctaccatcagagagcagttctggaaccaaccactcaaa
aagacaaccagcattcgatccatggaaaagtcctgaaaatattagtcattcagagcaactcaa
ggaaaaagagaagcaaggatttttcaggtcaatgaaaaagaaaaagaagaaatctcaaacagt
acccaattccgacagccctgatcttctgacgttgcagaaatccattcattctgctagcactcc
aagcagcagaccaaaggagtggcgccccgagaagatctcagatctgcagacccaaagccagcc
attaaaatcactgcgcaagttgttacatctctcttcggcctcaaatcacccggcttcctcaga
tccccgcttccagcccttaacagctcaacaaaccaaaaattccttctcagaaattcggattca
ccccctgagccaggcctctggcgggagcagcaacatccggcaggaacccgcaccgaagggcag
gccagccctccagctgccagacggtggatgtgatggcagaagacagagacaccattctggacc
ccaagatagacgcttcatgttaaggacgacagaacaacaaggagaatacttctgctgtggtga
cccaaagaagcctcacactccgtgcgtcccaaaccgagcccttcatcgtccaatctccagtcc
tgctccctatccagtactccaggtccgaggcacttccatgtgcccgacactccaggtccgagg
cactgatgctttcagctgcccaacccagcaatccgggttctctttcttcgtgagacacgttat
gagggaagccctgattcacagggcccaggtaaaccaagctgcgctcctgacataccatgagaa
tgcggcactgacgggcaagtccgctcgaggagggcccgaacaaaaactcatctcagaagagga
tctgaatagcgccgtcgaccatcatcatcatcatcattga
CDKL5 115 isoform fusion protein (Igκ-TATκ-eGFP-CDKL5(115)-
MYC-HIS) polypeptide. Translated from nucleotide 11 (initiator
methionine) of SEQ ID NO: 9.
SEQ ID NO: 10
METDTLLLWVLLLWVPGSTGDAAQPARRARRTKLAAYARKAARQARAPVATMVSKGEELFTGV
VPILVELDGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDFKEDGNIL
GHKLEYNYNSHNVYIMADKQKNGIKVNFKIRHNIEDGSVQLADHYQQNTPIGDGPVLLPDNHY
LSTQSALSKDPNEKRDHMVLLEFVTAAGITLGMDELYKSGLRSRAKIPNIGNVMNKFEILGVV
GEGAYGVVLKCRHKETHEIVAIKKFKDSEENEEVKETTLRELKMLRTLKQENIVELKEAFRRR
GKLYLVFEYVEKNMLELLEEMPNGVPPEKVKSYIYQLIKAIHWCHKNDIVHRDIKPENLLISH
NDVLKLCDFGFARNLSEGNNANYTEYVATRWYRSPELLLGAPYGKSVDMWSVGCILGELSDGQ
PLFPGESEIDQLFTIQKVLGPLPSEQMKLFYSNPRFHGLRFPAVNHPQSLERRYLGILNSVLL
DLMKNLLKLDPADRYLTEQCLNHPTFQTQRLLDRSPSRSAKRKPYHVESSTLSNRNQAGKSTA
LQSHHRSNSKDIQNLSVGLPRADEGLPANESFLNGNLAGASLSPLHTKTYQASSQPGSTSKDL
TNNNIPHLLSPKEAKSKTEFDFNIDPKPSEGPGTKYLKSNSRSQQNRHSFMESSQSKAGTLQP
NEKQSRHSYIDTIPQSSRSPSYRTKAKSHGALSDSKSVSNLSEARAQIAEPSTSRYFPSSCLD
LNSPTSPTPTRHSDTRTLLSPSGRNNRNEGTLDSRRTTTRHSKTMEELKLPEHMDSSHSHSLS
APHESFSYGLGYTSPFSSQQRPHRHSMYVTRDKVRAKGLDGSLSIGQGMAARANSLQLLSPQP
GEQLPPEMTVARSSVKETSREGTSSFHTRQKSEGGVYHDPHSDDGTAPKENRHLYNDPVPRRV
GSFYRVPSPRPDNSFHENNVSTRVSSLPSESSSGTNHSKRQPAFDPWKSPENISHSEQLKEKE
KQGFFRSMKKKKKKSQTVPNSDSPDLLTLQKSIHSASTPSSRPKEWRPEKISDLQTQSQPLKS
LRKLLHLSSASNHPASSDPRFQPLTAQQTKNSFSEIRIHPLSQASGGSSNIRQEPAPKGRPAL
QLPDGGCDGRRQRHHSGPQDRRFMLRTTEQQGEYFCCGDPKKPHTPCVPNRALHRPISSPAPY
PVLQVRGTSMCPTLQVRGTDAFSCPTQQSGFSFFVRHVMREALIHRAQVNQAALLTYHENAAL
TGKSARGGPEQKLISEEDLNSAVDHHHHHH
CDKL5 107 isoform fusion protein (Igκ-TATκ-eGFP-CDKL5(107)-MYC-
HIS) polynucleotide
SEQ ID NO: 11
atggagacagacacactcctgctatgggtactgctgctctgggttccaggttccactggtgac
gcggcccagccggccaggcgcgcgcgccgtacgaagcttgcggcctacgccagaaaggccgcc
aggcaggccagggcaccggtcgccaccatggtgagcaagggcgaggagctgttcaccggggtg
gtgcccatcctggtcgagctggacggcgacgtaaacggccacaagttcagcgtgtccggcgag
ggcgagggcgatgccacctacggcaagctgaccctgaagttcatctgcaccaccggcaagctg
cccgtgccctggcccaccctcgtgaccaccctgacctacggcgtgcagtgcttcagccgctac
cccgaccacatgaagcagcacgacttcttcaagtccgccatgcccgaaggctacgtccaggag
cgcaccatcttcttcaaggacgacggcaactacaagacccgcgccgaggtgaagttcgagggc
gacaccctggtgaaccgcatcgagctgaagggcatcgacttcaaggaggacggcaacatcctg
gggcacaagctggagtacaactacaacagccacaacgtctatatcatggccgacaagcagaag
aacggcatcaaggtgaacttcaagatccgccacaacatcgaggacggcagcgtgcagctcgcc
gaccactaccagcagaacacccccatcggcgacggccccgtgctgctgcccgacaaccactac
ctgagcacccagtccgccctgagcaaagaccccaacgagaagcgcgatcacatggtcctgctg
gagttcgtgaccgccgccgggatcactctcggcatggacgagctgtacaagtccggactcaga
tctcgagcgaagattcctaacattggtaatgtgatgaataaatttgagatccttggggttgta
ggtgaaggagcctatggagttgtacttaaatgcagacacaaggaaacacatgaaattgtggcg
atcaagaaattcaaggacagtgaagaaaatgaagaagtcaaagaaacgactttacgagagctt
aaaatgcttcggactctcaagcaggaaaacattgtggagttgaaggaagcatttcgtcggagg
ggaaagttgtacttggtgtttgagtatgttgaaaaaaatatgctcgaattgctggaagaaatg
ccaaatggagttccacctgagaaagtaaaaagctacatctatcagctaatcaaggctattcac
tggtgccataagaatgatattgtccatcgagatataaaaccagaaaatctcttaatcagccac
aatgatgtcctaaaactgtgtgactttggttttgctcgtaatctgtcagaaggcaataatgct
aattacacagagtacgttgccaccagatggtatcggtccccagaactcttacttggcgctccc
tatggaaagtccgtggacatgtggtcggtgggctgtattcttggggagcttagcgatggacag
cctttatttcctggagaaagtgaaattgaccaactttttactattcagaaggtgctaggacca
cttccatctgagcagatgaagcttttctacagtaatcctcgcttccatgggctccggtttcca
gctgttaaccatcctcagtccttggaaagaagataccttggaattttgaatagtgttctactt
gacctaatgaagaatttactgaagttggacccagctgacagatacttgacagaacagtgtttg
aatcaccctacatttcaaacccagagacttctggatcgttctccttcaaggtcagcaaaaaga
aaaccttaccatgtggaaagcagcacattgtctaatagaaaccaagccggcaaaagtactgct
ttgcagtctcaccacagatctaacagcaaggacatccagaacctgagtgtaggcctgccccgg
gctgacgaaggtctccctgccaatgaaagcttcctaaatggaaaccttgctggagctagtctt
agtccactgcacaccaaaacctaccaagcaagcagccagcctgggtctaccagcaaagatctc
accaacaacaacataccacaccttcttagcccaaaagaagccaagtcaaaaacagagtttgat
tttaatattgacccaaagccttcagaaggcccagggacaaagtacctcaagtcaaacagcaga
tctcagcagaaccgccactcattcatggaaagctctcaaagcaaagctgggacactgcagccc
aatgaaaagcagagtcggcatagctatattgacacaattccccagtcctctaggagtccctcc
tacaggaccaaggccaaaagccatggggcactgagtgactccaagtctgtgagcaacctttct
gaagccagggcccaaattgcggagcccagtaccagtaggtacttcccatctagctgcttagac
ttgaattctcccaccagcccaacccccaccagacacagtgacacgagaactttgctcagccct
tctggaagaaataaccgaaatgagggaacgctggactcacgtcgaaccacaaccagacattct
aagacgatggaggaattgaagctgccggagcacatggacagtagccattcccattcactgtct
gcacctcacgaatctttttcttatggactgggctacaccagccccttttcttcccagcaacgt
cctcataggcattctatgtatgtgacccgtgacaaagtgagagccaagggcttggatggaagc
ttgagcatagggcaagggatggcagctagagccaacagcctgcaactcttgtcaccccagcct
ggagaacagctccctccagagatgactgtggcaagatcttcggtcaaagagacctccagagaa
ggcacctcttccttccatacacgccagaagtctgagggtggagtgtatcatgacccacactct
gatgatggcacagcccccaaagaaaatagacacctatacaatgatcctgtgccaaggagagtt
ggtagcttttacagagtgccatctccacgtccagacaattctttccatgaaaataatgtgtca
actagagtttcttctctaccatcagagagcagttctggaaccaaccactcaaaaagacaacca
gcattcgatccatggaaaagtcctgaaaatattagtcattcagagcaactcaaggaaaaagag
aagcaaggatttttcaggtcaatgaaaaagaaaaagaagaaatctcaaacagtacccaattcc
gacagccctgatcttctgacgttgcagaaatccattcattctgctagcactccaagcagcaga
ccaaaggagtggcgccccgagaagatctcagatctgcagacccaaagccagccattaaaatca
ctgcgcaagttgttacatctctcttcggcctcaaatcacccggcttcctcagatccccgcttc
cagcccttaacagctcaacaaaccaaaaattccttctcagaaattcggattcaccccctgagc
caggcctctggcgggagcagcaacatccggcaggaacccgcaccgaagggcaggccagccctc
cagctgccaggtcagatggatcctggttggcatgtgtcctctgtgaccaggagtgccacagag
ggcccttcctactctgaacagctgggtgccaaaagtgggccaaatgggcacccctataacaga
acaaatcgctcacgaatgccaaatctgaatgatttaaaagagacagccttgtccgctcgagga
gggcccgaacaaaaactcatctcagaagaggatctgaatagcgccgtcgaccatcatcatcat
catcattga
CDKL5 107 isoform fusion protein (Igκ-TATκ-eGFP-CDKL5(107)-
MYC-HIS) polypeptide
SEQ ID NO: 12
METDTLLLWVLLLWVPGSTGDAAQPARRARRTKLAAYARKAARQARAPVATMVSKGEELFTGV
VPILVELDGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDFKEDGNIL
GHKLEYNYNSHNVYIMADKQKNGIKVNFKIRHNIEDGSVQLADHYQQNTPIGDGPVLLPDNHY
LSTQSALSKDPNEKRDHMVLLEFVTAAGITLGMDELYKSGLRSRAKIPNIGNVMNKFEILGVV
GEGAYGVVLKCRHKETHEIVAIKKFKDSEENEEVKETTLRELKMLRTLKQENIVELKEAFRRR
GKLYLVFEYVEKNMLELLEEMPNGVPPEKVKSYIYQLIKAIHWCHKNDIVHRDIKPENLLISH
NDVLKLCDFGFARNLSEGNNANYTEYVATRWYRSPELLLGAPYGKSVDMWSVGCILGELSDGQ
PLFPGESEIDQLFTIQKVLGPLPSEQMKLFYSNPRFHGLRFPAVNHPQSLERRYLGILNSVLL
DLMKNLLKLDPADRYLTEQCLNHPTFQTQRLLDRSPSRSAKRKPYHVESSTLSNRNQAGKSTA
LQSHHRSNSKDIQNLSVGLPRADEGLPANESFLNGNLAGASLSPLHTKTYQASSQPGSTSKDL
TNNNIPHLLSPKEAKSKTEFDFNIDPKPSEGPGTKYLKSNSRSQQNRHSFMESSQSKAGTLQP
NEKQSRHSYIDTIPQSSRSPSYRTKAKSHGALSDSKSVSNLSEARAQIAEPSTSRYFPSSCLD
LNSPTSPTPTRHSDTRTLLSPSGRNNRNEGTLDSRRTTTRHSKTMEELKLPEHMDSSHSHSLS
APHESFSYGLGYTSPFSSQQRPHRHSMYVTRDKVRAKGLDGSLSIGQGMAARANSLQLLSPQP
GEQLPPEMTVARSSVKETSREGTSSFHTRQKSEGGVYHDPHSDDGTAPKENRHLYNDPVPRRV
GSFYRVPSPRPDNSFHENNVSTRVSSLPSESSSGTNHSKRQPAFDPWKSPENISHSEQLKEKE
KQGFFRSMKKKKKKSQTVPNSDSPDLLTLQKSIHSASTPSSRPKEWRPEKISDLQTQSQPLKS
LRKLLHLSSASNHPASSDPRFQPLTAQQTKNSFSEIRIHPLSQASGGSSNIRQEPAPKGRPAL
QLPGQMDPGWHVSSVTRSATEGPSYSEQLGAKSGPNGHPYNRTNRSRMPNLNDLKETALSARG
GPEQKLISEEDLNSAVDHHHHHH
CDKL5 107 isoform fusion protein (Igk-TATκ-CDKL5(107)-3XFLAG
polynucleotide. Underline indicates codon for the initiator
methionine.
SEQ ID NO: 13
gctagccaccatggagacagacacactcctgctatgggtactgctgctctgggttccaggttc
cactggtgacgcggcccagccggccaggcgcgcgcgccgtacgaagcttgcggcctacgccag
aaaggccgccaggcaggccagggcaccggtgaagattcctaacattggtaatgtgatgaataa
atttgagatccttggggttgtaggtgaaggagcctatggagttgtacttaaatgcagacacaa
ggaaacacatgaaattgtggcgatcaagaaattcaaggacagtgaagaaaatgaagaagtcaa
agaaacgactttacgagagcttaaaatgcttcggactctcaagcaggaaaacattgtggagtt
gaaggaagcatttcgtcggaggggaaagttgtacttggtgtttgagtatgttgaaaaaaatat
gctcgaattgctggaagaaatgccaaatggagttccacctgagaaagtaaaaagctacatcta
tcagctaatcaaggctattcactggtgccataagaatgatattgtccatcgagatataaaacc
agaaaatctcttaatcagccacaatgatgtcctaaaactgtgtgactttggttttgctcgtaa
tctgtcagaaggcaataatgctaattacacagagtacgttgccaccagatggtatcggtcccc
agaactcttacttggcgctccctatggaaagtccgtggacatgtggtcggtgggctgtattct
tggggagcttagcgatggacagcctttatttcctggagaaagtgaaattgaccaactttttac
tattcagaaggtgctaggaccacttccatctgagcagatgaagcttttctacagtaatcctcg
cttccatgggctccggtttccagctgttaaccatcctcagtccttggaaagaagataccttgg
aattttgaatagtgttctacttgacctaatgaagaatttactgaagttggacccagctgacag
atacttgacagaacagtgtttgaatcaccctacatttcaaacccagagacttctggatcgttc
tccttcaaggtcagcaaaaagaaaaccttaccatgtggaaagcagcacattgtctaatagaaa
ccaagccggcaaaagtactgctttgcagtctcaccacagatctaacagcaaggacatccagaa
cctgagtgtaggcctgccccgggctgacgaaggtctccctgccaatgaaagcttcctaaatgg
aaaccttgctggagctagtcttagtccactgcacaccaaaacctaccaagcaagcagccagcc
tgggtctaccagcaaagatctcaccaacaacaacataccacaccttcttagcccaaaagaagc
caagtcaaaaacagagtttgattttaatattgacccaaagccttcagaaggcccagggacaaa
gtacctcaagtcaaacagcagatctcagcagaaccgccactcattcatggaaagctctcaaag
caaagctgggacactgcagcccaatgaaaagcagagtcggcatagctatattgacacaattcc
ccagtcctctaggagtccctcctacaggaccaaggccaaaagccatggggcactgagtgactc
caagtctgtgagcaacctttctgaagccagggcccaaattgcggagcccagtaccagtaggta
cttcccatctagctgcttagacttgaattctcccaccagcccaacccccaccagacacagtga
cacgagaactttgctcagcccttctggaagaaataaccgaaatgagggaacgctggactcacg
tcgaaccacaaccagacattctaagacgatggaggaattgaagctgccggagcacatggacag
tagccattcccattcactgtctgcacctcacgaatctttttcttatggactgggctacaccag
ccccttttcttcccagcaacgtcctcataggcattctatgtatgtgacccgtgacaaagtgag
agccaagggcttggatggaagcttgagcatagggcaagggatggcagctagagccaacagcct
gcaactcttgtcaccccagcctggagaacagctccctccagagatgactgtggcaagatcttc
ggtcaaagagacctccagagaaggcacctcttccttccatacacgccagaagtctgagggtgg
agtgtatcatgacccacactctgatgatggcacagcccccaaagaaaatagacacctatacaa
tgatcctgtgccaaggagagttggtagcttttacagagtgccatctccacgtccagacaattc
tttccatgaaaataatgtgtcaactagagtttcttctctaccatcagagagcagttctggaac
caaccactcaaaaagacaaccagcattcgatccatggaaaagtcctgaaaatattagtcattc
agagcaactcaaggaaaaagagaagcaaggatttttcaggtcaatgaaaaagaaaaagaagaa
atctcaaacagtacccaattccgacagccctgatcttctgacgttgcagaaatccattcattc
tgctagcactccaagcagcagaccaaaggagtggcgccccgagaagatctcagatctgcagac
ccaaagccagccattaaaatcactgcgcaagttgttacatctctcttcggcctcaaatcaccc
ggcttcctcagatccccgcttccagcccttaacagctcaacaaaccaaaaattccttctcaga
aattcggattcaccccctgagccaggcctctggcgggagcagcaacatccggcaggaacccgc
accgaagggcaggccagccctccagctgccaggtcagatggatcctggttggcatgtgtcctc
tgtgaccaggagtgccacagagggcccttcctactctgaacagctgggtgccaaaagtgggcc
aaatgggcacccctataacagaacaaatcgctcacgaatgccaaatctgaatgatttaaaaga
gacagccttgtctagaggatcccgggctgactacaaagaccatgacggtgattataaagatca
tgacatcgactacaaggatgacgatgacaagtag
CDKL5 107 isoform fusion protein (Igκ-TATκ-CDKL5(107)-3XFLAG
polynucleotide
SEQ ID NO: 14
METDTLLLWVLLLWVPGSTGDAAQPARRARRTKLAAYARKAARQARAPVKIPNIGNVMNKFEI
LGVVGEGAYGVVLKCRHKETHEIVAIKKFKDSEENEEVKETTLRELKMLRTLKQENIVELKEA
FRRRGKLYLVFEYVEKNMLELLEEMPNGVPPEKVKSYIYQLIKAIHWCHKNDIVHRDIKPENL
LISHNDVLKLCDFGFARNLSEGNNANYTEYVATRWYRSPELLLGAPYGKSVDMWSVGCILGEL
SDGQPLFPGESEIDQLFTIQKVLGPLPSEQMKLFYSNPRFHGLRFPAVNHPQSLERRYLGILN
SVLLDLMKNLLKLDPADRYLTEQCLNHPTFQTQRLLDRSPSRSAKRKPYHVESSTLSNRNQAG
KSTALQSHHRSNSKDIQNLSVGLPRADEGLPANESFLNGNLAGASLSPLHTKTYQASSQPGST
SKDLTNNNIPHLLSPKEAKSKTEFDFNIDPKPSEGPGTKYLKSNSRSQQNRHSFMESSQSKAG
TLQPNEKQSRHSYIDTIPQSSRSPSYRTKAKSHGALSDSKSVSNLSEARAQIAEPSTSRYFPS
SCLDLNSPTSPTPTRHSDTRTLLSPSGRNNRNEGTLDSRRTTTRHSKTMEELKLPEHMDSSHS
HSLSAPHESFSYGLGYTSPFSSQQRPHRHSMYVTRDKVRAKGLDGSLSIGQGMAARANSLQLL
SPQPGEQLPPEMTVARSSVKETSREGTSSFHTRQKSEGGVYHDPHSDDGTAPKENRHLYNDPV
PRRVGSFYRVPSPRPDNSFHENNVSTRVSSLPSESSSGTNHSKRQPAFDPWKSPENISHSEQL
KEKEKQGFFRSMKKKKKKSQTVPNSDSPDLLTLQKSIHSASTPSSRPKEWRPEKISDLQTQSQ
PLKSLRKLLHLSSASNHPASSDPRFQPLTAQQTKNSFSEIRIHPLSQASGGSSNIRQEPAPKG
RPALQLPGQMDPGWHVSSVTRSATEGPSYSEQLGAKSGPNGHPYNRTNRSRMPNLNDLKETAL
SRGSRADYKDHDGDYKDHDIDYKDDDDK
CDKL5 107 isoform polynucleotide. Sequence lacks the codon for
the initiator methionine.
SEQ ID NO: 15
aagattcctaacattggtaatgtgatgaataaatttgagatccttggggttgtaggtgaagga
gcctatggagttgtacttaaatgcagacacaaggaaacacatgaaattgtggcgatcaagaaa
ttcaaggacagtgaagaaaatgaagaagtcaaagaaacgactttacgagagcttaaaatgctt
cggactctcaagcaggaaaacattgtggagttgaaggaagcatttcgtcggaggggaaagttg
tacttggtgtttgagtatgttgaaaaaaatatgctcgaattgctggaagaaatgccaaatgga
gttccacctgagaaagtaaaaagctacatctatcagctaatcaaggctattcactggtgccat
aagaatgatattgtccatcgagatataaaaccagaaaatctcttaatcagccacaatgatgtc
ctaaaactgtgtgactttggttttgctcgtaatctgtcagaaggcaataatgctaattacaca
gagtacgttgccaccagatggtatcggtccccagaactcttacttggcgctccctatggaaag
tccgtggacatgtggtcggtgggctgtattcttggggagcttagcgatggacagcctttattt
cctggagaaagtgaaattgaccaactttttactattcagaaggtgctaggaccacttccatct
gagcagatgaagcttttctacagtaatcctcgcttccatgggctccggtttccagctgttaac
catcctcagtccttggaaagaagataccttggaattttgaatagtgttctacttgacctaatg
aagaatttactgaagttggacccagctgacagatacttgacagaacagtgtttgaatcaccct
acatttcaaacccagagacttctggatcgttctccttcaaggtcagcaaaaagaaaaccttac
catgtggaaagcagcacattgtctaatagaaaccaagccggcaaaagtactgctttgcagtct
caccacagatctaacagcaaggacatccagaacctgagtgtaggcctgccccgggctgacgaa
ggtctccctgccaatgaaagcttcctaaatggaaaccttgctggagctagtcttagtccactg
cacaccaaaacctaccaagcaagcagccagcctgggtctaccagcaaagatctcaccaacaac
aacataccacaccttcttagcccaaaagaagccaagtcaaaaacagagtttgattttaatatt
gacccaaagccttcagaaggcccagggacaaagtacctcaagtcaaacagcagatctcagcag
aaccgccactcattcatggaaagctctcaaagcaaagctgggacactgcagcccaatgaaaag
cagagtcggcatagctatattgacacaattccccagtcctctaggagtccctcctacaggacc
aaggccaaaagccatggggcactgagtgactccaagtctgtgagcaacctttctgaagccagg
gcccaaattgcggagcccagtaccagtaggtacttcccatctagctgcttagacttgaattct
cccaccagcccaacccccaccagacacagtgacacgagaactttgctcagcccttctggaaga
aataaccgaaatgagggaacgctggactcacgtcgaaccacaaccagacattctaagacgatg
gaggaattgaagctgccggagcacatggacagtagccattcccattcactgtctgcacctcac
gaatctttttcttatggactgggctacaccagccccttttcttcccagcaacgtcctcatagg
cattctatgtatgtgacccgtgacaaagtgagagccaagggcttggatggaagcttgagcata
gggcaagggatggcagctagagccaacagcctgcaactcttgtcaccccagcctggagaacag
ctccctccagagatgactgtggcaagatcttcggtcaaagagacctccagagaaggcacctct
tccttccatacacgccagaagtctgagggtggagtgtatcatgacccacactctgatgatggc
acagcccccaaagaaaatagacacctatacaatgatcctgtgccaaggagagttggtagcttt
tacagagtgccatctccacgtccagacaattctttccatgaaaataatgtgtcaactagagtt
tcttctctaccatcagagagcagttctggaaccaaccactcaaaaagacaaccagcattcgat
ccatggaaaagtcctgaaaatattagtcattcagagcaactcaaggaaaaagagaagcaagga
tttttcaggtcaatgaaaaagaaaaagaagaaatctcaaacagtacccaattccgacagccct
gatcttctgacgttgcagaaatccattcattctgctagcactccaagcagcagaccaaaggag
tggcgccccgagaagatctcagatctgcagacccaaagccagccattaaaatcactgcgcaag
ttgttacatctctcttcggcctcaaatcacccggcttcctcagatccccgcttccagccctta
acagctcaacaaaccaaaaattccttctcagaaattcggattcaccccctgagccaggcctct
ggcgggagcagcaacatccggcaggaacccgcaccgaagggcaggccagccctccagctgcca
ggtcagatggatcctggttggcatgtgtcctctgtgaccaggagtgccacagagggcccttcc
tactctgaacagctgggtgccaaaagtgggccaaatgggcacccctataacagaacaaatcgc
tcacgaatgccaaatctgaatgatttaaaagagacagccttg
CDKL5 107 isoform polypeptide. Sequence lacks the initiator
methionine.
SEQ ID NO: 16
KIPNIGNVMNKFEILGVVGEGAYGVVLKCRHKETHEIVAIKKFKDSEENEEVKETTLRELKML
RTLKQENIVELKEAFRRRGKLYLVFEYVEKNMLELLEEMPNGVPPEKVKSYIYQLIKAIHWCH
KNDIVHRDIKPENLLISHNDVLKLCDFGFARNLSEGNNANYTEYVATRWYRSPELLLGAPYGK
SVDMWSVGCILGELSDGQPLFPGESEIDQLFTIQKVLGPLPSEQMKLFYSNPRFHGLRFPAVN
HPQSLERRYLGILNSVLLDLMKNLLKLDPADRYLTEQCLNHPTFQTQRLLDRSPSRSAKRKPY
HVESSTLSNRNQAGKSTALQSHHRSNSKDIQNLSVGLPRADEGLPANESFLNGNLAGASLSPL
HTKTYQASSQPGSTSKDLTNNNIPHLLSPKEAKSKTEFDFNIDPKPSEGPGTKYLKSNSRSQQ
NRHSFMESSQSKAGTLQPNEKQSRHSYIDTIPQSSRSPSYRTKAKSHGALSDSKSVSNLSEAR
AQIAEPSTSRYFPSSCLDLNSPTSPTPTRHSDTRTLLSPSGRNNRNEGTLDSRRTTTRHSKTM
EELKLPEHMDSSHSHSLSAPHESFSYGLGYTSPFSSQQRPHRHSMYVTRDKVRAKGLDGSLSI
GQGMAARANSLQLLSPQPGEQLPPEMTVARSSVKETSREGTSSFHTRQKSEGGVYHDPHSDDG
TAPKENRHLYNDPVPRRVGSFYRVPSPRPDNSFHENNVSTRVSSLPSESSSGTNHSKRQPAFD
PWKSPENISHSEQLKEKEKQGFFRSMKKKKKKSQTVPNSDSPDLLTLQKSIHSASTPSSRPKE
WRPEKISDLQTQSQPLKSLRKLLHLSSASNHPASSDPRFQPLTAQQTKNSFSEIRIHPLSQAS
GGSSNIRQEPAPKGRPALQLPGQMDPGWHVSSVTRSATEGPSYSEQLGAKSGPNGHPYNRTNR
SRMPNLNDLKETAL

1-18. (canceled)

19. A method of treating a CDKL5 deficiency or a Rett syndrome variant, the method comprising systemically administering to a patient in need thereof a fusion protein comprising:
a CDKL5 polypeptide sequence, wherein the CDKL5 polypeptide sequence has about 98% to 100% sequence identity to SEQ ID NO:2 or SEQ ID NO: 16; and
a TATκ polypeptide sequence, wherein the TATκ polypeptide sequence has about 90% to about 100% sequence identity to SEQ ID NO: 4, wherein the TATκ polypeptide is operatively coupled to the CDKL5 polypeptide.

20. The method according to claim 19, wherein the fusion protein further comprises an Igk-chain leader sequence polypeptide, wherein the Igk-chain leader sequence is operatively coupled to the CDKL5 polypeptide.

21. The method according to claim 19, wherein the fusion protein further comprises a reporter protein polypeptide, wherein the reporter protein polypeptide is operatively coupled to the CDKL5 polypeptide.

22. The method according to claim 19, wherein the fusion protein further comprises a protein tag polypeptide, wherein the protein tag polypeptide is operatively coupled to the CDKL5 polypeptide.

23. The method according to claim 19, wherein the fusion protein has a polypeptide sequence according to SEQ ID NO: 8, SEQ ID NO: 10, SEQ ID NO: 12, or SEQ ID NO: 14.

24. The method according to claim 19, wherein systemic administration of the fusion protein increases neurite growth, elongation, dendritic spine number, branch number, or branch density in a brain of a subject as compared to a control.

25. The method according to claim 19, wherein systemic administration of the fusion protein reduces neuronal apoptosis in the brain of a subject as compared to a control.

26. The method according to claim 19, wherein the fusion protein is administered intravenously.

27. A method of increasing neural activity in the visual cortex of a patient having a CDKL5 deficiency or a Rett syndrome variant, the method comprising systemically administering to a patient in need thereof a fusion protein comprising:
a CDKL5 polypeptide sequence, wherein the CDKL5 polypeptide sequence has about 98% to 100% sequence identity to SEQ ID NO:2 or SEQ ID NO: 16; and
a TATκ polypeptide sequence, wherein the TATκ polypeptide sequence has about 90% to about 100% sequence identity to SEQ ID NO: 4, wherein the TATκ polypeptide is operatively coupled to the CDKL5 polypeptide.

28. The method according to claim 27, wherein the fusion protein further comprises an Igk-chain leader sequence polypeptide, wherein the Igk-chain leader sequence is operatively coupled to the CDKL5 polypeptide.

29. The method according to claim 27, wherein the fusion protein further comprises a reporter protein polypeptide, wherein the reporter protein polypeptide is operatively coupled to the CDKL5 polypeptide.

30. The method according to claim 27, wherein the fusion protein further comprises a protein tag polypeptide, wherein the protein tag polypeptide is operatively coupled to the CDKL5 polypeptide.

31. The method according to claim 27, wherein the fusion protein has a polypeptide sequence according to SEQ ID NO: 8, SEQ ID NO: 10, SEQ ID NO: 12, or SEQ ID NO: 14.

32. The method according to claim 27, wherein systemic administration of the fusion protein increases neurite growth, elongation, dendritic spine number, branch number, or branch density in a brain of a subject as compared to a control.

33. The method according to claim 27, wherein systemic administration of the fusion protein reduces neuronal apoptosis in the brain of a subject as compared to a control.

34. The method according to claim 27, wherein the fusion protein is administered intravenously.

35. A method of increasing neurite growth, elongation, dendritic spine number, branch number, or branch density in a brain of a patient having a CDKL5 deficiency or a Rett syndrome variant, the method comprising systemically administering to a patient in need thereof a fusion protein comprising:
a CDKL5 polypeptide sequence, wherein the CDKL5 polypeptide sequence has about 98% to 100% sequence identity to SEQ ID NO:2 or SEQ ID NO: 16; and
a TATκ polypeptide sequence, wherein the TATκ polypeptide sequence has about 90% to about 100% sequence identity to SEQ ID NO: 4, wherein the TATκ polypeptide is operatively coupled to the CDKL5 polypeptide.

36. The method according to claim 35, wherein the fusion protein further comprises an Igk-chain leader sequence polypeptide, wherein the Igk-chain leader sequence is operatively coupled to the CDKL5 polypeptide.

37. The method according to claim 35, wherein the fusion protein further comprises a reporter protein polypeptide, wherein the reporter protein polypeptide is operatively coupled to the CDKL5 polypeptide.

38. The method according to claim 35, wherein the fusion protein further comprises a protein tag polypeptide, wherein the protein tag polypeptide is operatively coupled to the CDKL5 polypeptide.

39-55. (canceled)


SEQUENCE LISTING (16 sequences)

SEQ ID NO: 1 - DNA, 3087 bp, Artificial Sequence
  CDKL5 115 isoform cDNA
aagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaag

SEQ ID NO: 2 - protein, 1029 aa, Artificial Sequence
  CDKL5 isoform 115 polypeptide
KIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGK

SEQ ID NO: 3 - DNA, 33 bp, Artificial Sequence
  TATk polynucleotide
tacgccagaaaggccgccaggcaggccagggca

SEQ ID NO: 4 - protein, 11 aa, Artificial Sequence
  TATk polypeptide
YARKAARQARA

SEQ ID NO: 5 - DNA, 60 bp, Artificial Sequence
  Igk-chain leader polynucleotide
atggagacagacacactcctgctatgggtactgctgctctgggttccaggttccactggt

SEQ ID NO: 6 - protein, 20 aa, Artificial Sequence
  Igk-chain leader polypeptide
METDTLLLWVLLLWVPGSTG

SEQ ID NO: 7 - DNA, 3318 bp, Artificial Sequence
  CDKL5 115 isoform fusion protein polynucleotide
atggagacagacacactcctgctatgggtactgctgctctgggttccaggttccactggtgacgcggcccagccggccaggcgcgcgcgccgtacgaagcttgcggcctacgccagaaaggccgccaggcaggccagggcaccggtgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaagtccgctcgaggagggcccgaacaaaaactcatctcagaagaggatctgaatagcgccgtcgaccatcatcatcatcatcattga

SEQ ID NO: 8 - protein, 1105 aa, Artificial Sequence [sequence omitted]

SEQ ID NO: 9 - DNA, 4072 bp, Artificial Sequence
  CDKL5 115 isoform fusion protein polynucleotide
  misc_feature (11)..(13): methionine initiator codon
gctagccaccatggagacagacacactcctgctatgggtactgctgctctgggttccaggttccactggtgacgcggcccagccggccaggcgcgcgcgccgtacgaagcttgcggcctacgccagaaaggccgccaggcaggccagggcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagcgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaagtccgctcgaggagggcccgaacaaaaactcatctcagaagaggatctgaatagcgccgtcgaccatcatcatcatcatcattga

SEQ ID NO: 10 - protein, 1353 aa, Artificial Sequence [sequence omitted]

SEQ ID NO: 11 - DNA, 3852 bp, Artificial Sequence
  CDKL5 107 isoform fusion protein polynucleotide
atggagacagacacactcctgctatgggtactgctgctctgggttccaggttccactggtgacgcggcccagccggccaggcgcgcgcgccgtacgaagcttgcggcctacgccagaaaggccgccaggcaggccagggcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagcgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccaggtcagatggatcctggttggcatgtgtcctctgtgaccaggagtgccacagagggcccttcctactctgaacagctgggtgccaaaagtgggccaaatgggcacccctataacagaacaaatcgctcacgaatgccaaatctgaatgatttaaaagagacagccttgtccgctcgaggagggcccgaacaaaaactcatctcagaagaggatctgaatagcgccgtcgaccatcatcatcatcatcattga

SEQ ID NO: 12 - protein, 1283 aa, Artificial Sequence [sequence omitted]

SEQ ID NO: 13 - DNA, 3120 bp, Artificial Sequence
  CDKL5 107 isoform fusion protein polynucleotide
  misc_feature (11)..(13): methionine initiator codon
ctagccaccatggagacagacacactcctgctatgggtactgctgctctgggttccaggttccactggtgacgcggcccagccggccaggcgcgcgcgccgtacgaagcttgcggcctacgccagaaaggccgccaggcaggccagggcaccggtgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccaggtcagatggatcctggttggcatgtgtcctctgtgaccaggagtgccacagagggcccttcctactctgaacagctgggtgccaaaagtgggccaaatgggcacccctataacagaacaaatcgctcacgaatgccaaatctgaatgatttaaaagagacagccttgtctagaggatcccgggctgactacaaagaccatgacggtgattataaagatcatgacatcgactacaaggatgacgatgacaagtag

SEQ ID NO: 14 - protein, 1036 aa, Artificial Sequence
  CDKL5 107 isoform fusion protein polypeptide
METDTLLLWVLLLWVPGSTGDAAQPARRARRTKLAAYARKAARQARAPVKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALSRGSRADYKDHDGDYKDHDIDYKDDDDK

SEQ ID NO: 15 - DNA, 2877 bp, Artificial Sequence
  CDKL5 107 isoform polynucleotide
aagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccaggtcagatggatcctggttggcatgtgtcctctgtgaccaggagtgccacagagggcccttcctactctgaacagctgggtgccaaaagtgggccaaatgggcacccctataacagaacaaatcgctcacgaatgccaaatctgaatgatttaaaagagacagccttg

SEQ ID NO: 16 - protein, 959 aa, Artificial Sequence
  CDKL5 107 isoform polypeptide
KIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL